(19) United States
(12) Patent Application Publication
(10) Pub. No.: US 2019/0352616 A1
(43) Pub. Date: Nov. 21, 2019

(54) MULTIPLE TRANSGENE RECOMBINANT ADENOVIRUS

(71) Applicant: EpicentRx, Inc., La Jolla, CA (US)

(72) Inventors: Tony R. Reid, San Diego, CA (US); Bryan T. Oronsky, Los Altos Hills, CA (US); Christopher Larson, San Diego, CA (US)

(21) Appl. No.: 16/482,055

(22) PCT Filed: Jan. 30, 2018
(86) PCT No.: PCT/US2018/016032

§ 371 Date: Jul. 30, 2019

Related U.S. Application Data
(60) Provisional application No. 62/520,945, filed on Jun. 16, 2017.
(60) Provisional application No. 62/452,342, filed on Jan. 30, 2017.

(51) Int. Cl. C12N 7/00; A61K 35/761; C07K 14/705; A61P 35/00

(52) U.S. Cl. CPC C12N 7/00; A61K 35/761; C07K 14/70532; C07K 14/70575; C12N 2710/10343; A61P 35/00; C12N 2710/10321; C12N 2710/10332; C12N 2840/203; C07K 14/70525

(57) ABSTRACT

The invention provides a recombinant adenovirus comprising two (or more) therapeutic transgenes, e.g., CD80 and CD137L. The transgenes are preferably inserted into an E1b-19K insertion site and/or an E3 insertion site.

### CROSS-REFERENCE TO RELATED APPLICATIONS

This application claims the benefit of, and priority to, U.S. Provisional Patent Application Ser. No. 62/452,342, filed Jan. 30, 2017 and U.S. Provisional Patent Application Ser. No. 62/520,945, filed Jun. 16, 2017.

### FIELD OF THE INVENTION

The field of the invention is molecular biology and virology, specifically modified viruses that express two or more therapeutic transgenes.

### BACKGROUND

Despite extensive knowledge of the underlying molecular mechanisms that cause cancer, most advanced cancers remain incurable with current chemotherapy and radiation protocols. Oncolytic viruses have emerged as a platform technology that has the potential to significantly augment current standard treatment for a variety of malignancies (Kumar, S. et al. (2008) CURRENT OPINION IN MOLECULAR THERAPEUTICS 10(4):371-379; Kim, D. (2001) EXPERT OPINION ON BIOLOGICAL THERAPY 1(3):525-538; Kim D. (2000) ONCOGENE 19(56):6660-6669). These viruses have shown promise as oncolytic agents that not only directly destroy malignant cells via an infection-to-reproduction-to-lysis chain reaction but also indirectly induce anti-tumor immunity. These immune stimulatory properties have been augmented with the insertion of therapeutic transgenes that are copied and expressed each time the virus replicates.

Previously developed oncolytic viruses include the oncolytic serotype 5 adenovirus (Ad5) referred to as TAV-255 that is transcriptionally attenuated in normal cells but transcriptionally active in cancer cells (see, PCT Publication No. WO2010/101921). It is believed that the mechanism by which the TAV-255 vector achieves this tumor selectivity is through targeted deletion of three transcriptional factor (TF) binding sites for the transcription factors Pea3 and E2F, proteins that regulate adenovirus expression of E1a, the earliest gene to be transcribed after virus entry into the host cell, through binding to specific DNA sequences.

Despite the efforts to date, there is a need for improved oncolytic viruses for treating cancers and hyperproliferative disorders in human patients.

### SUMMARY OF THE INVENTION

The invention is based, in part, upon the discovery that adenoviruses such as oncolytic viruses, unexpectedly can efficiently express, when inserted into particular insertion sites, multiple (two or more) therapeutic transgenes without the use of an exogenous promoter and that the viruses can replicate and efficiently express the two or more therapeutic transgenes despite the size of the transgenes incorporated into the viral genome.

Accordingly, in one aspect the invention provides a recombinant adenovirus comprising: (a) a first nucleotide sequence encoding a first therapeutic transgene inserted into an E1b-19K insertion site; wherein the E1b-19K insertion site is located between the start site of E1b-19K and the start site of E1b-55K; and (b) a second nucleotide sequence encoding a second therapeutic transgene inserted into an E3 insertion site, wherein the E3 insertion site is located between the stop site of pVIII and the start site of Fiber.

In certain embodiments, the recombinant adenovirus is a type 5 adenovirus (Ad5).

In certain embodiments, the E1b-19K insertion site is located between the start site of E1b-19K and the stop site of E1b-19K. In certain embodiments, the E1b-19K insertion site comprises a deletion of from about 100 to about 305, about 100 to about 300, about 100 to about 250, about 100 to about 200, about 100 to about 150, about 150 to about 305, about 150 to about 300, about 150 to about 250, or about 150 to about 200 nucleotides adjacent the start site of E1b-19K. In certain embodiments, the E1b-19K insertion site comprises a deletion of about 200 nucleotides, e.g., 202 or 203 nucleotides adjacent the start site of E1b-19K. In certain embodiments, the E1b-19K insertion site comprises a deletion corresponding to nucleotides 1714-1917 or 1714-1916 of the Ad5 genome (SEQ ID NO: 23). In certain embodiments, the first therapeutic transgene is inserted between nucleotides corresponding to 1714 and 1917 or between nucleotides corresponding to 1714 and 1916 of the Ad5 genome (SEQ ID NO: 23). In certain embodiments, the first therapeutic transgene is inserted between CTGACCTC (SEQ ID NO: 1) and TCACCAGG (SEQ ID NO: 2), e.g., the recombinant adenovirus comprises, in a 5′ to 3′ orientation, CTGACCTC (SEQ ID NO: 1), the first therapeutic transgene, and TCACCAGG (SEQ ID NO: 2).

In certain embodiments, the E3 insertion site comprises a deletion of from about 500 to about 3185, from about 500 to about 3000, from about 500 to about 2500, from about 500 to about 2000, from about 500 to about 1500, from about 500 to about 1000, from about 1000 to about 3185, from about 1000 to about 3000, from about 1000 to about 2500, from about 1000 to about 2000, from about 1000 to about 1500, from about 1500 to about 3185, from about 1500 to about 3000, from about 1500 to about 2000, from about 2000 to about 3185, from about 2000 to about 3000, from about 2000 to about 2500, from about 2500 to about 3185, from about 2500 to about 3000, or from about 3000 to about 3185 nucleotides. In certain embodiments, the E3 insertion site is located between the stop site of E3-10.5K and the stop site of E3-14.7K. In certain embodiments, the E3 insertion site comprises a deletion of from about 500 to about 1551, from about 500 to about 1500, from about 500 to about 1000, from about 1000 to about 1551, from about 1000 to about 1500, or from about 1500 to about 1551 nucleotides adjacent the stop site of E3-10.5K. In certain embodiments, the E3 insertion site comprises a deletion of about 1050 nucleotides adjacent the stop site of E3-10.5K, e.g., the E3 insertion site comprises a deletion of 1063 or 1064 nucleotides adjacent the stop site of E3-10.5K. In certain embodiments, the E3 insertion site comprises a deletion corresponding to the Ad5 dl309 E3 deletion. In certain embodiments, the E3 insertion site comprises a deletion corresponding to nucleotides 29773-30836 of the Ad5 genome (SEQ ID NO: 23). In certain embodiments, the second therapeutic transgene is inserted between nucleotides corresponding to 29773 and 30836 of the Ad5 genome (SEQ ID NO: 23). In certain embodiments, the second therapeutic transgene is inserted between CAGTATGA (SEQ ID NO: 3) and TAATAAAAAA (SEQ ID NO: 4), e.g., the recombinant adenovirus comprises, in a 5′ to 3′ orientation, CAGTATGA (SEQ ID NO: 3), the second therapeutic transgene, and TAATAAAAAA (SEQ ID NO: 4). In certain embodiments, the E3 insertion site is located between stop site of E3-gp19K and the stop site of E3-14.7K. In certain embodiments, the E3 insertion site comprises a deletion of from about 500 to about 1824, from about 500 to about 1500, from about 500 to about 1000, from about 1000 to about 1824, from about 1000 to about 1500, or from about 1500 to about 1824 nucleotides adjacent the stop site of E3-gp19K. In certain embodiments, the E3 insertion site comprises a deletion of about 1600 nucleotides adjacent the stop site of E3-gp19K. e.g., the E3 insertion site comprises a deletion of 1622 nucleotides adjacent the stop site of E3-gp19K. In certain embodiments, the E3 insertion site comprises a deletion corresponding to nucleotides 29218-30839 of the Ad5 genome (SEQ ID NO: 23). In certain embodiments, the second therapeutic transgene is inserted between nucleotides corresponding to 29218 and 30839 of the Ad5 genome (SEQ ID NO: 23). In certain embodiments, the second therapeutic transgene is inserted between TGCCTTAA (SEQ ID NO: 29) and TAAAAAAAAAT (SEQ ID NO: 30), e.g., the recombinant adenovirus comprises, in a 5′ to 3′ orientation, TGCCTTAA (SEQ ID NO: 29), the second therapeutic transgene, and TAAAAAAAAAT (SEQ ID NO: 30).

In another aspect, the invention provides a recombinant adenovirus comprising: (a) a first nucleotide sequence encoding a first therapeutic transgene inserted into an E1b-19k insertion site; and (b) a second nucleotide sequence encoding a second therapeutic transgene inserted into the E1b-19k insertion site, wherein the E1b-19k insertion site is located between the start site of E1b-19k and the start site of E1b-55k, and wherein the first nucleotide sequence and the second nucleotide sequence are separated by a first internal ribosome entry site (IRES).

In certain embodiments, the recombinant adenovirus is a type 5 adenovirus (Ad5).

In certain embodiments, the E1b-19K insertion site is located between the start site of E1b-19K and the stop site of E1b-19K. In certain embodiments, the E1b-19K insertion site comprises a deletion of from about 100 to about 305, about 100 to about 300, about 100 to about 250, about 100 to about 200, about 100 to about 150, about 150 to about 305, about 150 to about 300, about 150 to about 250, or about 150 to about 200 nucleotides adjacent the start site of E1b-19K. In certain embodiments, the E1b-19K insertion site comprises a deletion of about 200 nucleotides, e.g., 202 or 203 nucleotides adjacent the start site of E1b-19K. In certain embodiments, the E1b-19K insertion site comprises a deletion corresponding to nucleotides 1714-1917 or 1714-1916 of the Ad5 genome (SEQ ID NO: 23). In certain embodiments, the first and second therapeutic transgenes are inserted between nucleotides corresponding to 1714 and 1917 or between nucleotides corresponding to 1714 and 1916 of the Ad5 genome (SEQ ID NO: 23). In certain embodiments, the first and second therapeutic transgenes are inserted between CTGACCTC (SEQ ID NO: 1) and TCACCAGG (SEQ ID NO: 2), e.g., the recombinant adenovirus comprises, in a 5′ to 3′ orientation, CTGACCTC (SEQ ID NO: 1), the first therapeutic transgene, the first IRES, the second therapeutic transgene, and TCACCAGG (SEQ ID NO: 2).

In certain embodiments the recombinant adenovirus comprises an E3 deletion. In certain embodiments, the E3 deletion comprises a deletion of from about 500 to about 3185, from about 500 to about 3000, from about 500 to about 2500, from about 500 to about 2000, from about 500 to about 1500, from about 500 to about 1000, from about 1000 to about 3185, from about 1000 to about 3000, from about 1000 to about 2500, from about 1000 to about 2000, from about 1000 to about 1500, from about 1500 to about 3185, from about 1500 to about 3000, from about 1500 to about 2000, from about 2000 to about 3185, from about 2000 to about 3000, from about 2000 to about 2500, from about 2500 to about 3185, from about 2500 to about 3000, or from about 3000 to about 3185 nucleotides. In certain embodiments, the E3 deletion site is located between the stop site of pVIII and the start site of Fiber. In certain embodiments, the E3 deletion site is located between the stop site of E3-10.5K and the stop site of E3-14.7K. In certain embodiments, the E3 deletion comprises a deletion of from about 500 to about 1551, from about 500 to about 1500, from about 500 to about 1000, from about 1000 to about 1551, from about 1000 to about 1500, or from about 1500 to about 1551 nucleotides adjacent the stop site of E3-10.5K. In certain embodiments, the E3 deletion comprises a deletion of about 1050 nucleotides adjacent the stop site of E3-10.5K, e.g., the E3 deletion comprises a deletion of 1063 or 1064 nucleotides adjacent the stop site of E3-10.5K. In certain embodiments, the E3 deletion comprises a deletion corresponding to the Ad5 dl309 E3 deletion. In certain embodiments, the E3 deletion comprises a deletion corresponding to nucleotides 29773-30836 of the Ad5 genome (SEQ ID NO: 23). In certain embodiments, the E3 deletion is located between stop site of E3-gp19K and the stop site of E3-14.7K. In certain embodiments, the E3 deletion comprises a deletion of from about 500 to about 1824, from about 500 to about 1500, from about 500 to about 1000, from about 1000 to about 1824, from about 1000 to about 1500, or from about 1500 to about 1824 nucleotides adjacent the stop site of E3-gp19K. In certain embodiments, the E3 deletion comprises a deletion of about 1600 nucleotides adjacent the stop site of E3-gp19K. e.g., the E3 insertion site comprises a deletion of 1622 nucleotides adjacent the stop site of E3-gp19K. In certain embodiments, the E3 deletion comprises a deletion corresponding to nucleotides 29218-30839 of the Ad5 genome (SEQ ID NO: 23).

In certain embodiments, the recombinant adenovirus comprises a third nucleotide sequence encoding a third therapeutic transgene. The third therapeutic transgene may be inserted into the E1b-19k insertion site wherein, e.g., the second nucleotide sequence and the third nucleotide sequence are separated by a second internal ribosome entry site (IRES). In certain embodiments, the first, second, and third therapeutic transgenes are inserted between CTGACCTC (SEQ ID NO: 1) and TCACCAGG (SEQ ID NO: 2), e.g., the recombinant adenovirus comprises, in a 5′ to 3′ orientation, CTGACCTC (SEQ ID NO: 1), the first therapeutic transgene, the first IRES, the second therapeutic transgene, the second IRES, the third therapeutic transgene, and TCACCAGG (SEQ ID NO: 2). The third therapeutic transgene may also be inserted into the E3 deletion site, i.e., in certain embodiments the recombinant adenovirus comprises a third nucleotide sequence encoding a third therapeutic transgene inserted into an E3 insertion site. In certain embodiments, the third therapeutic transgene is inserted between nucleotides corresponding to 29773 and 30836 of the Ad5 genome. In certain embodiments, the third therapeutic transgene is inserted between CAGTATGA (SEQ ID NO: 3) and TAATAAAAAA (SEQ ID NO: 4), e.g., the recombinant adenovirus comprises, in a 5′ to 3′ orientation, CAGTATGA (SEQ ID NO: 3), the third therapeutic transgene, and TAATAAAAAA (SEQ ID NO: 4). In certain embodiments, the third therapeutic transgene is inserted between nucleotides corresponding to 29218 and 30839 of the Ad5 genome (SEQ ID NO: 23). In certain embodiments, the third therapeutic transgene is inserted between TGCCTTAA (SEQ ID NO: 29) and TAAAAAAAAAT (SEQ ID NO: 30), e.g., the recombinant adenovirus comprises, in a 5′ to 3′ orientation, TGCCTTAA (SEQ ID NO: 29), the third therapeutic transgene, and TAAAAAAAAAT (SEQ ID NO: 30).

The IRES may, e.g., be selected from the group consisting of the encephalomyocarditis virus (EMCV) IRES, the foot-and-mouth disease virus (FMDV) IRES, and the poliovirus IRES.

In certain embodiments, in any of the foregoing viruses, the recombinant adenovirus further comprises an E4 deletion. In certain embodiments, the E4 deletion is located between the start site of E4-ORF6/7 and the right inverted terminal repeat (ITR). In certain embodiments, the E4 deletion is located between the start site of E4-ORF6/7 and the start site of E4-ORF1. In certain embodiments, the E4 deletion comprises a deletion of from about 500 to about 2500, from about 500 to about 2000, from about 500 to about 1500, from about 500 to about 1000, from about 1000 to about 2500, from about 1000 to about 2000, from about 1000 to about 1500, from about 1500 to about 2500, from about 1500 to about 2000, or from about 2000 to about 2500 nucleotides. In certain embodiments, the E4 deletion comprises a deletion of from about 250 to about 1500, from about 250 to about 1250, from about 250 to about 1000, from about 250 to about 750, from about 250 to about 500, from 500 to about 1500, from about 500 to about 1250, from about 500 to about 1000, from about 500 to about 750, from 750 to about 1500, from about 750 to about 1250, from about 750 to about 1000, from about 1000 to about 1500, or from about 1000 to about 1250 nucleotides adjacent the start site of E4-ORF6/7. In certain embodiments, the E4 deletion comprises a deletion of about 1450 nucleotides adjacent the start site of E4-ORF6/7, e.g., the E4 deletion comprises a deletion of about 1449 nucleotides adjacent the start site of E4-ORF6/7. In certain embodiments, the E4 deletion comprises a deletion corresponding to nucleotides 34078-35526 of the Ad5 genome (SEQ ID NO: 23).

In certain embodiments, in any of the foregoing viruses, the first and/or second therapeutic transgenes, the first, second, and/or third therapeutic transgenes, or all of the therapeutic transgenes, are not operably linked to an exogenous promoter sequence.

In certain embodiments, the size of the first and second therapeutic transgenes, the size of the first, second, and third therapeutic transgenes, or the size of all of the therapeutic transgenes, when combined, comprise from about 500 to about 5000, from about 500 to about 4000, from about 500 to about 3000, from about 500 to about 2000, from about 500 to about 1000, from about 1000 to about 5000, from about 1000 to about 4000, from about 1000 to about 3000, from about 1000 to about 2000, from about 2000 to about 5000, from about 2000 to about 4000, from about 2000 to about 3000, from about 3000 to about 5000, from about 3000 to about 4000, or from about 4000 to about 5000 nucleotides. In certain embodiments, the size of the first and second therapeutic transgenes, the size of the first, second, and third therapeutic transgenes, or the size of all of the therapeutic transgenes, when combined, comprise from about 500 to about 7000, from about 500 to about 6000, from about 500 to about 5000, from about 500 to about 4000, from about 500 to about 3000, from about 500 to about 2000, from about 500 to about 1000, from about 1000 to about 7000, from about 1000 to about 6000, from about 1000 to about 5000, from about 1000 to about 4000, from about 1000 to about 3000, from about 1000 to about 2000, from about 2000 to about 7000, from about 2000 to about 6000, from about 2000 to about 5000, from about 2000 to about 4000, from about 2000 to about 3000, from about 3000 to about 7000, from about 3000 to about 6000, from about 3000 to about 5000, from about 3000 to about 4000, from about 4000 to about 7000, from about 4000 to about 6000, from about 4000 to about 5000 nucleotides, from about 5000 to about 7000, from about 5000 to about 6000, or from about 6000 to about 7000 nucleotides.

In certain embodiments, the size of the first and second therapeutic transgenes, the size of the first, second, and third therapeutic transgenes, or the size of all of the therapeutic transgenes, when combined, comprise at least about 500, about 1000, about 2000, about 3000, about 4000, or about 5000 nucleotides. In certain embodiments, the size of the first and second therapeutic transgenes, the size of the first, second, and third therapeutic transgenes, or the size of all of the therapeutic transgenes, when combined, comprise about 1650 nucleotides. In certain embodiments, the size of the first and second therapeutic transgenes, the size of the first, second, and third therapeutic transgenes, or the size of all of the therapeutic transgenes, when combined, comprise at least about 500, about 1000, about 2000, about 3000, about 4000, about 5000, about 6000, or about 7000 nucleotides. In certain embodiments, the size of the first and second therapeutic transgenes, the size of the first, second, and third therapeutic transgenes, or the size of all of the therapeutic transgenes, when combined, comprise about 3100 nucleotides.

In certain embodiments, in any of the foregoing viruses, the first and/or second therapeutic transgene, the first, second, and/or third therapeutic transgenes, or any of the therapeutic transgenes encode a therapeutic polypeptide selected from the group consisting of CD80, CD137L, IL-23A/p19, p40, endostatin, angiostatin, ICAM-1, and a TGF-β trap.

In certain embodiments, in any of the foregoing viruses, the first and/or second therapeutic transgene, the first, second, and/or third therapeutic transgenes, or any of the therapeutic transgenes encode a therapeutic polypeptide selected from the group consisting of CD80, CD137L, IL-23, IL-23A/p19, p40, IL-27, IL-27A/p28, IL-27B/EBI3, endostatin, angiostatin, ICAM-1, a TGF-β trap, TGF-β, CD19, CD20, IL-1, IL-3, IL-4, IL-5, IL-6, IL-8, IL-9, CD154, CD86, BORIS/CTCFL, FGF, IL-24, MAGE, NY-ESO-1, acetylcholine, interferon-gamma, DKK1/Wnt, p53, thymidine kinase, an anti-PD-1 antibody heavy chain or light chain, and an anti-PD-L1 antibody heavy chain or light chain.

In certain embodiments, the first and second therapeutic transgene encode a first and second subunit, respectively, of a heterodimeric cytokine.

In certain embodiments, in any of the foregoing viruses, the first and/or second therapeutic transgenes are selected from the group consisting of CD80 and CD137L, e.g., the first therapeutic transgene encodes CD80 and the second therapeutic transgene encodes CD137L. In certain embodiments, the recombinant adenovirus comprises a nucleotide sequence encoding an amino acid sequence that is encoded by SEQ ID NO: 5, and/or SEQ ID NO: 7, or comprises the nucleotide sequence of SEQ ID NO: 6, and/or SEQ ID NO: 8. In certain embodiments, the recombinant adenovirus comprises the nucleotide sequence of SEQ ID NO: 27.

In certain embodiments, in any of the foregoing viruses, the first, second, and/or third therapeutic transgenes are selected from the group consisting of CD80, CD137L, and ICAM-1, e.g., the first therapeutic transgene encodes CD80, the second therapeutic transgene encodes CD137L, and the third therapeutic transgene encodes ICAM-1. In certain embodiments, the recombinant adenovirus comprises a nucleotide sequence encoding an amino acid sequence that is encoded by SEQ ID NO: 5, SEQ ID NO: 7, and/or SEQ ID NO: 32. In certain embodiments, the recombinant adenovirus comprises the nucleotide sequence of SEQ ID NO: 31, SEQ ID NO: 9, or SEQ ID NO: 22.

In certain embodiments, in any of the foregoing viruses, the first and/or second therapeutic transgenes are selected from the group consisting of IL-23A/p19 and p40, which make up the heterodimeric cytokine IL-23. For example, in certain embodiments, the first therapeutic transgene encodes IL-23A/p19 and the second therapeutic transgene encodes p40. In certain embodiments, the recombinant adenovirus comprises a nucleotide sequence encoding an amino acid sequence that is encoded by SEQ ID NO: 12 and/or SEQ ID NO: 10, or comprises the nucleotide sequence of SEQ ID NO: 13.

In certain embodiments, in any of the foregoing viruses, the first and/or second therapeutic transgenes are selected from the group consisting of IL-27A/p28 and IL-27B/EBI3, which make up the heterodimeric cytokine IL-27. For example, in certain embodiments, the first therapeutic transgene encodes IL-27A/p28 and the second therapeutic transgene encodes IL-27B/EBI3.

In certain embodiments, in any of the foregoing viruses, the first and/or second therapeutic transgenes are selected from the group consisting of endostatin and angiostatin e.g., the first therapeutic transgene encodes endostatin and the second therapeutic transgene encodes angiostatin. In certain embodiments, the recombinant adenovirus comprises a nucleotide sequence encoding the amino acid sequence of SEQ ID NO: 37 or SEQ ID NO: 38. In certain embodiments, the recombinant adenovirus comprises a nucleotide sequence encoding the amino acid sequence of SEQ ID NO: 39, SEQ ID NO: 40, SEQ ID NO: 41, SEQ ID NO: 42, SEQ ID NO: 43 or SEQ ID NO: 44. In certain embodiments, the recombinant adenovirus comprises the nucleotide sequence of SEQ ID NO: 11.

In certain embodiments, any of the foregoing recombinant adenoviruses may comprise a deletion of at least one Pea3 binding site, or a functional portion thereof, e.g., the virus may comprise a deletion of nucleotides corresponding to about −300 to about −250 upstream of the initiation site of E1a or a deletion of nucleotides corresponding to −305 to −255 or −304 to −255 upstream of the initiation site of E1a.

In certain embodiments, in any of the foregoing compositions, the recombinant oncolytic adenovirus may comprise a deletion of at least one E2F binding site, or a functional portion thereof. In certain embodiments, the recombinant oncolytic adenovirus may comprise a deletion of at least one E2F binding site, or a functional portion thereof, and not comprise a deletion of a Pea3 binding site.

In another aspect, the invention provides a recombinant adenovirus comprising SEQ ID NO: 14, or a sequence having 80%, 85%, 86%, 87%, 88%, 89%, 90%, 91%, 92%, 93%, 94%, 95%, 96%, 97%, 98%, or 99% sequence identity to SEQ ID NO: 14.

In certain embodiments, each of the foregoing recombinant adenoviruses may selectively replicate in a hyperproliferative cell. In certain embodiments, any of the foregoing recombinant adenoviruses may selectively express two or more therapeutic transgenes in a hyperproliferative cell. The hyperproliferative cell may be a cancer cell, e.g., a lung cancer cell, a colon cancer cell, and a pancreatic cancer cell. In certain embodiments, each of the foregoing recombinant adenoviruses may be an oncolytic adenovirus.

In another aspect, the invention provides a pharmaceutical composition comprising each of the foregoing recombinant adenoviruses and at least one pharmaceutically acceptable carrier or diluent.

In another aspect, the invention provides a method of treating cancer in a subject. The method comprises administering to the subject an effective amount of a recombinant adenovirus described herein to treat the cancer disease in the subject. In certain embodiments, the cancer is selected from the group consisting of melanoma, squamous cell carcinoma of the skin, basal cell carcinoma, head and neck cancer, breast cancer, anal cancer, cervical cancer, non-small cell lung cancer, mesothelioma, small cell lung cancer, renal cell carcinoma, prostate cancer, gastroesophageal cancer, colorectal cancer, testicular cancer, bladder cancer, ovarian cancer, hepatocellular carcinoma, cholangiocarcinoma, brain cancer, endometrial cancer, neuroendocrine cancer, merkel cell carcinoma, gastrointestinal stromal tumors, a sarcoma, and pancreatic cancer.

In another aspect, the invention provides a method of inhibiting proliferation of a tumor cell in a subject. The method comprises administering to the subject an effective amount of a recombinant adenovirus described herein to inhibit proliferation of the tumor cell.

In another aspect, the invention provides a method of inhibiting tumor growth in a subject. The method comprises administering to the subject an effective amount of a recombinant adenovirus described herein to inhibit proliferation of the tumor cell.

In each of the foregoing methods, the recombinant adenovirus can, e.g., be administered in combination with one or more therapies selected from the group consisting of surgery, radiation, chemotherapy, immunotherapy, hormone therapy, and virotherapy. In each of the foregoing methods, the effective amount of the recombinant adenovirus can be, e.g., 10²-10¹⁵ plaque forming units (pfus). In each of the foregoing methods, the subject can, e.g., be a human, e.g., a pediatric human, or an animal.

In each of the foregoing methods, the effective amount of the recombinant virus may, e.g., be identified by measuring an immune response to an antigen in the subject. In certain embodiments, the immune response to the antigen is measured by injecting the subject with the antigen at an injection site on the skin of the subject and measuring the size of an induration at the injection site.

In another aspect, the invention provides a method of expressing two or more therapeutic transgenes in a target cell. The method comprises exposing the cell to an effective amount of the recombinant adenovirus described herein to express the target transgenes.

These and other aspects and advantages of the invention are illustrated by the following figures, detailed description and claims.

### DESCRIPTION OF THE DRAWINGS

The invention can be more completely understood with reference to the following drawings.

FIG. 1 depicts staining of ADS-12 cells for mouse CD80 or mouse CD137L two days following infection with the indicated virus at a multiplicity of infection (MOI) of 5.

FIG. 2 depicts staining of ADS-12 cells for mouse CD80 or mouse CD137L two days following infection with the indicated virus at a multiplicity of infection (MOI) of 5.

FIG. 3 depicts staining of 4T1 cells for mouse CD80 or mouse CD137L three days following infection with the indicated virus.

FIG. 4 depicts staining of 4T1 cells for mouse CD80 or mouse CD137L three days following infection with the indicated virus.

FIG. 5 depicts staining of non-cancerous (WI-38 and MRC5) or cancerous (A549) cells for human CD80 or human CD137L two days following infection with the indicated virus at a MOI of 2.

FIG. 6 depicts staining of A549 cells for human CD80 or human CD137L two days following infection with the indicated virus at a MOI of 5.

FIG. 7 depicts crystal violet staining of non-cancerous (WI-38 and MRC5) or cancerous (A549) cells at the indicated timepoints with or without infection with the TAV-hCD80-hCD137L virus at a MOI of 10.

FIG. 8 depicts crystal violet staining of ADS-12 cells at the indicated timepoints with or without infection with the indicated virus at a MOI of 10.

FIG. 9 depicts replication of the indicated viruses in ADS cells as determined by plaque assays.

FIG. 10 depicts mean tumor volume (±SEM) of subcutaneous ADS-12 tumors in mice following treatment with three intratumoral injections of 5.10⁷ PFU of the indicated virus on days 0, 4, and 8 (n=10). Tumor volumes were estimated as length·width²/2.

FIG. 11 depicts tumor volumes of subcutaneous ADS-12 tumors in mice following treatment with three intratumoral injections of 1·10⁷ PFU of the indicated virus on days 0, 4, and 8 (n=3). Tumor volumes were estimated as length·width²/2.

FIG. 12 depicts mean tumor volume (±SEM) of orthotopic 4T1 tumors in the mammary fat pad of mice following treatment with three intratumoral injections of 5·10⁷ PFU of the indicated virus on days 0, 4, and 8 (n=10). Tumor volumes were estimated as length·width²/2.

FIG. 13 depicts staining of ADS-12 cells for murine CD80, murine CD137L, and murine ICAM-1 four days following infection with the indicated virus at a MOI of 10.

FIG. 14 depicts staining of F244 cells for murine CD80, murine CD137L, and murine ICAM-1 three days following infection with the indicated virus at a MOI of 5.

FIG. 15 depicts staining of HT29 cells for murine CD80, murine CD137L, and murine ICAM-1 three days following infection with the indicated virus at a MOI of 5.

FIG. 16 depicts tumor volumes of 129S4 mice carrying subcutaneous ADS-12 tumors treated with intratumoral injections of either buffer (FIG. 16A), TAV-mCD80-137L (FIG. 16B), or TAV-mCD80-137L-ICAM (FIG. 16C). Each treatment was dosed every four days at 1×10⁹ PFU per dose for a total of three doses. Each line represents the tumor volume of an individual mouse, with 10 mice per each treatment group.

### DETAILED DESCRIPTION

The invention is based, in part, upon the discovery that adenoviruses such as oncolytic viruses, unexpectedly can efficiently express, when inserted into particular insertion sites, multiple (two or more) therapeutic transgenes without the use of an exogenous promoter and that the viruses can replicate and efficiently express the two or more therapeutic transgenes despite the size of the transgenes incorporated into the viral genome.

Accordingly, in one aspect the invention provides a recombinant adenovirus comprising: (a) a first nucleotide sequence encoding a first therapeutic transgene inserted into an E1b-19K insertion site; wherein the E1b-19K insertion site is located between the start site of E1b-19K (i.e., the nucleotide sequence encoding the start codon of E1b-19k, e.g., corresponding to nucleotides 1714-1716 of SEQ ID NO: 23) and the start site of E1b-55K (i.e., the nucleotide sequence encoding the start codon of E1b-55k, e.g., corresponding to nucleotides 2019-2021 of SEQ ID NO: 23); and (b) a second nucleotide sequence encoding a second therapeutic transgene inserted into an E3 insertion site, wherein the E3 insertion site is located between the stop site of pVIII (i.e., the nucleotide sequence encoding the stop codon of pVIII, e.g., corresponding to nucleotides 27855-27857 of SEQ ID NO: 23) and the start site of Fiber (i.e., the nucleotide sequence encoding the start codon of Fiber, e.g., corresponding to nucleotides 31042-31044 of SEQ ID NO: 23). Throughout the description and claims, an insertion between two sites, for example, an insertion between (i) a start site of a first gene (e.g., E1b-19k) and a start site of a second gene, (e.g., E1b-55K), (ii) a start site of a first gene and a stop site of a second gene, (iii) a stop site of a first gene and start site of a second gene, or (iv) a stop site of first gene and a stop site of a second gene, is understood to mean that all or a portion of the nucleotides constituting a given start site or a stop site surrounding the insertion may be present or absent in the final virus. Similarly, an insertion between two nucleotides is understood to mean that the nucleotides surrounding the insertion may be present or absent in the final virus. The term “transgene” refers to an exogenous gene or polynucleotide sequence. The term “therapeutic transgene” refers to a transgene, which when replicated and/or expressed in or by the virus imparts a therapeutic effect in a target cell, body fluid, tissue, organ, physiological system, or subject.

In certain embodiments, the E1b-19K insertion site is located between the start site of E1b-19K (i.e., the nucleotide sequence encoding the start codon of E1b-19k, e.g., corresponding to nucleotides 1714-1716 of SEQ ID NO: 23) and the stop site of E1b-19K (i.e., the nucleotide sequence encoding the stop codon of E1b-19k, e.g., corresponding to nucleotides 2242-2244 of SEQ ID NO: 23). In certain embodiments, the E1b-19K insertion site comprises a deletion of from about 100 to about 305, about 100 to about 300, about 100 to about 250, about 100 to about 200, about 100 to about 150, about 150 to about 305, about 150 to about 300, about 150 to about 250, or about 150 to about 200 nucleotides adjacent the start site of E1b-19K. In certain embodiments, the E1b-19K insertion site comprises a deletion of about 200 nucleotides, e.g., 202 or 203 nucleotides adjacent the start site of E1b-19K. In certain embodiments, the E1b-19K insertion site comprises a deletion corresponding to nucleotides 1714-1917 or 1714-1916 of the Ad5 genome (SEQ ID NO: 23). In certain embodiments, the first therapeutic transgene is inserted between nucleotides corresponding to 1714 and 1917 or between nucleotides corresponding to 1714 and 1916 of the Ad5 genome (SEQ ID NO: 23). In certain embodiments, the first therapeutic transgene is inserted between CTGACCTC (SEQ ID NO: 1) and TCACCAGG (SEQ ID NO: 2), e.g., the recombinant adenovirus comprises, in a 5′ to 3′ orientation, CTGACCTC (SEQ ID NO: 1), the first therapeutic transgene, and TCACCAGG (SEQ ID NO: 2). CTGACCTC (SEQ ID NO: 1) and TCACCAGG (SEQ ID NO: 2) define unique boundary sequences for the E1b-19K insertion site within the Ad5 genome (SEQ ID NO: 23). Throughout the description and claims, a deletion adjacent to a site, for example, a deletion adjacent to a start site of a gene or a deletion adjacent to a stop site of a gene, is understood to mean that the deletion may include a deletion of all, a portion, or none of the nucleotides constituting a given start site or a stop site.

In certain embodiments, the E3 insertion site comprises a deletion of from about 500 to about 3185, from about 500 to about 3000, from about 500 to about 2500, from about 500 to about 2000, from about 500 to about 1500, from about 500 to about 1000, from about 1000 to about 3185, from about 1000 to about 3000, from about 1000 to about 2500, from about 1000 to about 2000, from about 1000 to about 1500, from about 1500 to about 3185, from about 1500 to about 3000, from about 1500 to about 2000, from about 2000 to about 3185, from about 2000 to about 3000, from about 2000 to about 2500, from about 2500 to about 3185, from about 2500 to about 3000, or from about 3000 to about 3185 nucleotides. In certain embodiments, the E3 insertion site is located between the stop site of E3-10.5K (i.e., the nucleotide sequence encoding the stop codon of E3-10.5K, e.g., corresponding to nucleotides 29770-29772 of SEQ ID NO: 23) and the stop site of E3-14.7K (i.e., the nucleotide sequence encoding the stop codon of E3-14.7K, e.g., corresponding to nucleotides 30837-30839 of SEQ ID NO: 23). In certain embodiments, the E3 insertion site comprises a deletion of from about 500 to about 1551, from about 500 to about 1500, from about 500 to about 1000, from about 1000 to about 1551, from about 1000 to about 1500, or from about 1500 to about 1551 nucleotides adjacent the stop site of E3-10.5K. In certain embodiments, the E3 insertion site comprises a deletion of about 1050 nucleotides adjacent the stop site of E3-10.5K, e.g., the E3 insertion site comprises a deletion of 1063 or 1064 nucleotides adjacent the stop site of E3-10.5K. In certain embodiments, the E3 insertion site comprises a deletion corresponding to the Ad5 dl309 E3 deletion. In certain embodiments, the E3 insertion site comprises a deletion corresponding to nucleotides 29773-30836 of the Ad5 genome (SEQ ID NO: 23). In certain embodiments, the second therapeutic transgene is inserted between nucleotides corresponding to 29773 and 30836 of the Ad5 genome (SEQ ID NO: 23). In certain embodiments, the second therapeutic transgene is inserted between CAGTATGA (SEQ ID NO: 3) and TAATAAAAAA (SEQ ID NO: 4), e.g., the recombinant adenovirus comprises, in a 5′ to 3′ orientation, CAGTATGA (SEQ ID NO: 3), the second therapeutic transgene, and TAATAAAAAA (SEQ ID NO: 4). CAGTATGA (SEQ ID NO: 3) and TAATAAAAAA (SEQ ID NO: 4) define unique boundary sequences for an E3 insertion site within the Ad5 genome (SEQ ID NO: 23).

In certain embodiments, the E3 insertion site is located between stop site of E3-gp19K (i.e., the nucleotide sequence encoding the stop codon of E3-gp19K, e.g., corresponding to nucleotides 29215-29217 of SEQ ID NO: 23) and the stop site of E3-14.7K (i.e., the nucleotide sequence encoding the stop codon of E3-14.7K, e.g., corresponding to nucleotides 30837-30839 of SEQ ID NO: 23). In certain embodiments, the E3 insertion site comprises a deletion of from about 500 to about 1824, from about 500 to about 1500, from about 500 to about 1000, from about 1000 to about 1824, from about 1000 to about 1500, or from about 1500 to about 1824 nucleotides adjacent the stop site of E3-gp19K. In certain embodiments, the E3 insertion site comprises a deletion of about 1600 nucleotides adjacent the stop site of E3-gp19K. e.g., the E3 insertion site comprises a deletion of 1622 nucleotides adjacent the stop site of E3-gp19K. In certain embodiments, the E3 insertion site comprises a deletion corresponding to nucleotides 29218-30839 of the Ad5 genome (SEQ ID NO: 23). In certain embodiments, the second therapeutic transgene is inserted between nucleotides corresponding to 29218 and 30839 of the Ad5 genome (SEQ ID NO: 23). In certain embodiments, the second therapeutic transgene is inserted between TGCCTTAA (SEQ ID NO: 29) and TAAAAAAAAAT (SEQ ID NO: 30), e.g., the recombinant adenovirus comprises, in a 5′ to 3′ orientation, TGCCTTAA (SEQ ID NO: 29), the second therapeutic transgene, and TAAAAAAAAAT (SEQ ID NO: 30). TGCCTTAA (SEQ ID NO: 29) and TAAAAAAAAAT (SEQ ID NO: 30) define unique boundary sequences for an E3 insertion site within the Ad5 genome (SEQ ID NO: 23).

In another aspect, the invention provides a recombinant adenovirus comprising: (a) a first nucleotide sequence encoding a first therapeutic transgene inserted into an E1b-19k insertion site; and (b) a second nucleotide sequence encoding a second therapeutic transgene inserted into the E1b-19k insertion site, wherein the E1b-19k insertion site is located between the start of E1b-19k (i.e., the nucleotide sequence encoding the start codon of E1b-19k, e.g., corresponding to nucleotides 1714-1716 of SEQ ID NO: 23) and the start site of E1b-55k (i.e., the nucleotide sequence encoding the start codon of E1b-55k, e.g., corresponding to nucleotides 2019-2021 of SEQ ID NO: 23), and wherein the first nucleotide sequence and the second nucleotide sequence are separated by a first internal ribosome entry site (IRES).

In certain embodiments, the E1b-19K insertion site is located between the start site of E1b-19K (i.e., the nucleotide sequence encoding the start codon of E1b-19k, e.g., corresponding to nucleotides 1714-1716 of SEQ ID NO: 23) and the stop site of E1b-19K (i.e., the nucleotide sequence encoding the stop codon of E1b-19k, e.g., corresponding to nucleotides 2242-2244 of SEQ ID NO: 23). In certain embodiments, the E1b-19K insertion site comprises a deletion of from about 100 to about 305, about 100 to about 300, about 100 to about 250, about 100 to about 200, about 100 to about 150, about 150 to about 305, about 150 to about 300, about 150 to about 250, or about 150 to about 200 nucleotides adjacent the start site of E1b-19K. In certain embodiments, the E1b-19K insertion site comprises a deletion of about 200 nucleotides, e.g., 202 or 203 nucleotides adjacent the start site of E1b-19K. In certain embodiments, the E1b-19K insertion site comprises a deletion corresponding to nucleotides 1714-1917 or 1714-1916 of the Ad5 genome (SEQ ID NO: 23). In certain embodiments, the first and second therapeutic transgenes are inserted between nucleotides corresponding to 1714 and 1917 or between nucleotides corresponding to 1714 and 1916 of the Ad5 genome. In certain embodiments, the first and second therapeutic transgenes are inserted between CTGACCTC (SEQ ID NO: 1) and TCACCAGG (SEQ ID NO: 2), e.g., the recombinant adenovirus comprises, in a 5′ to 3′ orientation, CTGACCTC (SEQ ID NO: 1), the first therapeutic transgene, the IRES, the second therapeutic transgene, and TCACCAGG (SEQ ID NO: 2).

In certain embodiments the recombinant adenovirus comprises an E3 deletion. In certain embodiments, the E3 deletion comprises a deletion of from about 500 to about 3185, from about 500 to about 3000, from about 500 to about 2500, from about 500 to about 2000, from about 500 to about 1500, from about 500 to about 1000, from about 1000 to about 3185, from about 1000 to about 3000, from about 1000 to about 2500, from about 1000 to about 2000, from about 1000 to about 1500, from about 1500 to about 3185, from about 1500 to about 3000, from about 1500 to about 2000, from about 2000 to about 3185, from about 2000 to about 3000, from about 2000 to about 2500, from about 2500 to about 3185, from about 2500 to about 3000, or from about 3000 to about 3185 nucleotides. In certain embodiments the E3 deletion is located between the stop site of pVIII (i.e., the nucleotide sequence encoding the stop codon of pVIII, e.g., corresponding to nucleotides 27855-27857 of SEQ ID NO: 23) and the start site of Fiber (i.e., the nucleotide sequence encoding the start codon of Fiber, e.g., corresponding to nucleotides 31042-31044 of SEQ ID NO: 23). In certain embodiments, the E3 deletion site is located between the stop site of E3-10.5K (i.e., the nucleotide sequence encoding the stop codon of E3-10.5K, e.g., corresponding to nucleotides 29770-29772 of SEQ ID NO: 23) and the stop site of E3-14.7K (i.e., the nucleotide sequence encoding the stop codon of E3-14.7K, e.g., corresponding to nucleotides 30837-30839 of SEQ ID NO: 23). In certain embodiments, the E3 deletion comprises a deletion of from about 500 to about 1551, from about 500 to about 1500, from about 500 to about 1000, from about 1000 to about 1551, from about 1000 to about 1500, or from about 1500 to about 1551 nucleotides adjacent the stop site of E3-10.5K. In certain embodiments, the E3 deletion comprises a deletion of about 1050 nucleotides adjacent the stop site of E3-10.5K, e.g., the E3 deletion comprises a deletion of 1063 or 1064 nucleotides adjacent the stop site of E3-10.5K. In certain embodiments, the E3 deletion comprises a deletion corresponding to the Ad5 dl309 E3 deletion. In certain embodiments, the E3 deletion comprises a deletion corresponding to nucleotides 29773-30836 of the Ad5 genome (SEQ ID NO: 23).

In certain embodiments, the E3 deletion is located between stop site of E3-gp19K (i.e., the nucleotide sequence encoding the stop codon of E3-gp19K, e.g., corresponding to nucleotides 29215-29217 of SEQ ID NO: 23) and the stop site of E3-14.7K (i.e., the nucleotide sequence encoding the stop codon of E3-14.7K, e.g., corresponding to nucleotides 30837-30839 of SEQ ID NO: 23). In certain embodiments, the E3 deletion comprises a deletion of from about 500 to about 1824, from about 500 to about 1500, from about 500 to about 1000, from about 1000 to about 1824, from about 1000 to about 1500, or from about 1500 to about 1824 nucleotides adjacent the stop site of E3-gp19K. In certain embodiments, the E3 deletion comprises a deletion of about 1600 nucleotides adjacent the stop site of E3-gp19K. e.g., the E3 deletion comprises a deletion of 1622 nucleotides adjacent the stop site of E3-gp19K. In certain embodiments, the E3 deletion comprises a deletion corresponding to nucleotides 29218-30839 of the Ad5 genome (SEQ ID NO: 23).

In certain embodiments, the recombinant adenovirus comprises a third nucleotide sequence encoding a third therapeutic transgene. The third therapeutic transgene may be inserted into the E1b-19k insertion site wherein, e.g., the second nucleotide sequence and the third nucleotide sequence are separated by a second internal ribosome entry site (IRES). In certain embodiments, the first, second, and third therapeutic transgenes are inserted between CTGACCTC (SEQ ID NO: 1) and TCACCAGG (SEQ ID NO: 2), e.g., the recombinant adenovirus comprises, in a 5′ to 3′ orientation, CTGACCTC (SEQ ID NO: 1), the first therapeutic transgene, the first IRES, the second therapeutic transgene, the second IRES, the third therapeutic transgene, and TCACCAGG (SEQ ID NO: 2). The third therapeutic transgene may also be inserted into the E3 deletion site, i.e., in certain embodiments the recombinant adenovirus comprises a third nucleotide sequence encoding a third therapeutic transgene inserted into an E3 insertion site. In certain embodiments, the third therapeutic transgene is inserted between nucleotides corresponding to 29772 and 30837 of the Ad5 genome (SEQ ID NO: 23). In certain embodiments, the third therapeutic transgene is inserted between CAGTATGA (SEQ ID NO: 3) and TAATAAAAAA (SEQ ID NO: 4), e.g., the recombinant adenovirus comprises, in a 5′ to 3′ orientation, CAGTATGA (SEQ ID NO: 3), the third therapeutic transgene, and TAATAAAAAA (SEQ ID NO: 4). In certain embodiments, the third therapeutic transgene is inserted between nucleotides corresponding to 29218 and 30839 of the Ad5 genome (SEQ ID NO: 23). In certain embodiments, the third therapeutic transgene is inserted between TGCCTTAA (SEQ ID NO: 29) and TAAAAAAAAAT (SEQ ID NO: 30), e.g., the recombinant adenovirus comprises, in a 5′ to 3′ orientation, TGCCTTAA (SEQ ID NO: 29), the third therapeutic transgene, and TAAAAAAAAAT (SEQ ID NO: 30).

The IRES may, e.g., be selected from the group consisting of the encephalomyocarditis virus IRES, the foot-and-mouth disease virus IRES, and the poliovirus IRES.

In certain embodiments, in any of the foregoing viruses, the recombinant adenovirus further comprises an E4 deletion. In certain embodiments, the E4 deletion is located between the start site of E4-ORF6/7 (i.e., the nucleotide sequence encoding the start codon of E4-ORF6/7, e.g., corresponding to nucleotides 34075-34077 of SEQ ID NO: 23) and the right inverted terminal repeat (ITR; e.g., corresponding to nucleotides 35836-35938 of SEQ ID NO: 23). In certain embodiments, the E4 deletion is located between the start site of E4-ORF6/7 and the start site of E4-ORF1 (i.e., the nucleotide sequence encoding the start codon of E4-ORF1, e.g., corresponding to nucleotides 35524-35526 of SEQ ID NO: 23). In certain embodiments, the E4 deletion comprises a deletion of a nucleotide sequence between the start site of E4-ORF6/7 and the start site of E4-ORF1. In certain embodiments, the E4 deletion comprises a deletion of from about 500 to about 2500, from about 500 to about 2000, from about 500 to about 1500, from about 500 to about 1000, from about 1000 to about 2500, from about 1000 to about 2000, from about 1000 to about 1500, from about 1500 to about 2500, from about 1500 to about 2000, or from about 2000 to about 2500 nucleotides. In certain embodiments, the E4 deletion comprises a deletion of from about 250 to about 1500, from about 250 to about 1250, from about 250 to about 1000, from about 250 to about 750, from about 250 to about 500, from 500 to about 1500, from about 500 to about 1250, from about 500 to about 1000, from about 500 to about 750, from 750 to about 1500, from about 750 to about 1250, from about 750 to about 1000, from about 1000 to about 1500, or from about 1000 to about 1250 nucleotides adjacent the start site of E4-ORF6/7. In certain embodiments, the E4 deletion comprises a deletion of about 1450 nucleotides adjacent the start site of E4-ORF6/7, e.g., the E4 deletion comprises a deletion of about 1449 nucleotides adjacent the start site of E4-ORF6/7. In certain embodiments, the E4 deletion comprises a deletion corresponding to nucleotides 34078-35526 of the Ad5 genome (SEQ ID NO: 23).

In certain embodiments, a recombinant adenovirus of the invention is an oncolytic virus, e.g., a virus that exhibits tumor-selective replication and/or viral mediated lysis. In certain embodiments, a recombinant adenovirus of the invention exhibits selective expression of a therapeutic transgene in a hyperproliferative cell, e.g., a cancer cell, relative to a non-hyperproliferative cell. In certain embodiments, the expression of a therapeutic transgene in a non-hyperproliferative cell is about 90%, about 80%, about 70%, about 60%, about 50%, about 40%, about 30%, about 20%, about 10%, or about 5% of the expression of the gene in the hyperproliferative cell. In certain embodiments, the virus exhibits no detectable expression of a therapeutic transgene in a non-hyperproliferative cell. Therapeutic transgene expression may be determined by any appropriate method known in the art, e.g., Western blot or ELISA.

The hyperproliferative cell may be a cancer cell, e.g., a carcinoma, sarcoma, leukemia, lymphoma, prostate cancer, lung cancer, gastrointestinal tract cancer, colorectal cancer, pancreatic cancer, breast cancer, ovarian cancer, cervical cancer, stomach cancer, thyroid cancer, mesothelioma, liver cancer, kidney cancer, skin cancer, head and neck cancer, or brain cancer cell.

Features of recombinant adenoviruses of the invention, e.g., the lack of exogenous promoters, may allow for the expression of additional therapeutic transgenes or larger therapeutic transgenes relative to other recombinant adenoviruses. For example, in certain embodiments, in any of the foregoing viruses, the first and/or second therapeutic transgenes, the first, second, and/or third therapeutic transgenes, or all of the therapeutic transgenes are not operably linked to an exogenous promoter sequence. In certain embodiments, the size of the first and second therapeutic transgenes, the size of the first, second, and third therapeutic transgenes, or the size of all of the therapeutic transgenes, when combined, comprise from about 500 to about 5000, from about 500 to about 4000, from about 500 to about 3000, from about 500 to about 2000, from about 500 to about 1000, from about 1000 to about 5000, from about 1000 to about 4000, from about 1000 to about 3000, from about 1000 to about 2000, from about 2000 to about 5000, from about 2000 to about 4000, from about 2000 to about 3000, from about 3000 to about 5000, from about 3000 to about 4000, or from about 4000 to about 5000 nucleotides. In certain embodiments, the size of the first and second therapeutic transgenes, the size of the first, second, and third therapeutic transgenes, or the size of all of the therapeutic transgenes, when combined, comprise from about 500 to about 7000, from about 500 to about 6000, from about 500 to about 5000, from about 500 to about 4000, from about 500 to about 3000, from about 500 to about 2000, from about 500 to about 1000, from about 1000 to about 7000, from about 1000 to about 6000, from about 1000 to about 5000, from about 1000 to about 4000, from about 1000 to about 3000, from about 1000 to about 2000, from about 2000 to about 7000, from about 2000 to about 6000, from about 2000 to about 5000, from about 2000 to about 4000, from about 2000 to about 3000, from about 3000 to about 7000, from about 3000 to about 6000, from about 3000 to about 5000, from about 3000 to about 4000, from about 4000 to about 7000, from about 4000 to about 6000, from about 4000 to about 5000 nucleotides, from about 5000 to about 7000, from about 5000 to about 6000, or from about 6000 to about 7000 nucleotides.

In certain embodiments, the size of the first and second therapeutic transgenes, the size of the first, second, and third therapeutic transgenes, or the size of all of the therapeutic transgenes, when combined, comprise at least about 500, about 1000, about 2000, about 3000, about 4000, or about 5000 nucleotides. In certain embodiments, the size of the first and second therapeutic transgenes, the size of the first, second, and third therapeutic transgenes, or the size of all of the therapeutic transgenes, when combined, comprise about 1650 nucleotides. In certain embodiments, the size of the first and second therapeutic transgenes, the size of the first, second, and third therapeutic transgenes, or the size of all of the therapeutic transgenes, when combined, comprise at least about 500, about 1000, about 2000, about 3000, about 4000, about 5000, about 6000, or about 7000 nucleotides. In certain embodiments, the size of the first and second therapeutic transgenes, the size of the first, second, and third therapeutic transgenes, or the size of all of the therapeutic transgenes, when combined, comprise about 3100 nucleotides.

In certain embodiments, the recombinant adenovirus comprises SEQ ID NO: 14, or comprises a sequence having 80%, 85%, 86%, 87%, 88%, 89%, 90%, 91%, 92%, 93%, 94%, 95%, 96%, 97%, 98%, or 99% sequence identity to SEQ ID NO: 14.

Sequence identity may be determined in various ways that are within the skill in the art, e.g., using publicly available computer software such as BLAST, BLAST-2, ALIGN or Megalign (DNASTAR) software. BLAST (Basic Local Alignment Search Tool) analysis using the algorithm employed by the programs blastp, blastn, blastx, tblastn and tblastx (Karlin et al., (1990) PROC. NATL. ACAD. SCI. USA 87:2264-2268; Altschul, (1993) J. MOL. EVOL. 36, 290-300; Altschul et al., (1997) NUCLEIC ACIDS RES. 25:3389-3402, incorporated by reference) are tailored for sequence similarity searching. For a discussion of basic issues in searching sequence databases see Altschul et al., (1994) NATURE GENETICS 6:119-129, which is fully incorporated by reference. Those skilled in the art can determine appropriate parameters for measuring alignment, including any algorithms needed to achieve maximal alignment over the full length of the sequences being compared. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., (1992) PROC. NATL. ACAD. SCI. USA 89:10915-10919, fully incorporated by reference). Four blastn parameters may be adjusted as follows: Q=10 (gap creation penalty); R=10 (gap extension penalty); wink=1 (generates word hits at every wink.sup.th position along the query); and gapw=16 (sets the window width within which gapped alignments are generated). The equivalent Blastp parameter settings may be Q=9; R=2; wink=1; and gapw=32. Searches may also be conducted using the NCBI (National Center for Biotechnology Information) BLAST Advanced Option parameter (e.g.: −G, Cost to open gap [Integer]: default=5 for nucleotides/11 for proteins; −E, Cost to extend gap [Integer]: default=2 for nucleotides/1 for proteins; −q, Penalty for nucleotide mismatch [Integer]: default=−3; −r, reward for nucleotide match [Integer]: default=1; −e, expect value [Real]: default=10; −W, wordsize [Integer]: default=11 for nucleotides/28 for megablast/3 for proteins; −y, Dropoff (X) for blast extensions in bits: default=20 for blastn/7 for others; −X, X dropoff value for gapped alignment (in bits): default=15 for all programs, not applicable to blastn; and −Z, final X dropoff value for gapped alignment (in bits): 50 for blastn, 25 for others). ClustalW for pairwise protein alignments may also be used (default parameters may include, e.g., Blosum62 matrix and Gap Opening Penalty=10 and Gap Extension Penalty=0.1). A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

The invention also provides an adenovirus type 5 vector that expresses one or more therapeutic transgenes, in particular, immunomodulatory transgenes in E1, E3 and E4 sites, and right and left orientations. As used herein “immunomodulatory” refers to a therapeutic transgene that modulates the function of the immune system of a subject. Immunomodulatory transgenes may modulate the function of, e.g., B-cells, T cells and/or the production of antibodies. Exemplary immunomodulatory transgenes include checkpoint inhibitors. Exemplary immunomodulatory transgenes may include, e.g., PD-1, or PD-L1, or any transgene that modulates the activity thereof. Further exemplary immunomodulatory transgenes may include an anti PD-1 antibody, or anti-PD-L1 antibody. Certain immunomodulatory transgenes may comprise peptide linkers, e.g., peptide linkers from 2 to 5000 or more amino acids in length that may be immunogenic, i.e., that are vulnerable to neutralizing antibodies. It is contemplated that the immunogenicity of such linkers may be reduced by replacing the immunogenic sequences with non-immunogenic sequences.

The invention further provides methods of treatment comprising administering a disclosed recombinant adenovirus in combination with antibodies that, e.g., block immune checkpoints or improve antigen presentation/engulfment of antigens and/or/enhance tumor-specific T-cell responsiveness.

### I. Viruses

The term “virus” is used herein to refer any of the obligate intracellular parasites having no protein-synthesizing or energy-generating mechanism. The viral genome may be RNA or DNA. The viruses useful in the practice of the present invention include recombinantly modified enveloped or non-enveloped DNA and RNA viruses, preferably selected from baculoviridiae, parvoviridiae, picornoviridiae, herpesviridiae, poxyiridae, or adenoviridiae. A recombinantly modified virus is referred to herein as a “recombinant virus.” A recombinant virus may, e.g., be modified by recombinant DNA techniques to be replication deficient, conditionally replicating, or replication competent, and/or be modified by recombinant DNA techniques to include expression of exogenous transgenes. Chimeric viral vectors which exploit advantageous elements of each of the parent vector properties (See, e.g., Feng et al. (1997) NATURE BIOTECHNOLOGY 15:866-870) may also be useful in the practice of the present invention. Although it is generally favored to employ a virus from the species to be treated, in some instances it may be advantageous to use vectors derived from different species that possess favorable pathogenic features. For example, equine herpes virus vectors for human gene therapy are described in PCT Publication No. WO 98/27216. The vectors are described as useful for the treatment of humans as the equine virus is not pathogenic to humans. Similarly, ovine adenoviral vectors may be used in human gene therapy as they are claimed to avoid the antibodies against the human adenoviral vectors. Such vectors are described in PCT Publication No. WO 97/06826.

Preferably, the recombinant virus is an adenovirus. Adenoviruses are medium-sized (90-100 nm), non-enveloped (naked), icoshedral viruses composed of a nucleocapsid and a double-stranded linear DNA genome. Adenoviruses replicate in the nucleus of mammalian cells using the host's replication machinery. The term “adenovirus” refers to any virus in the genus Adenoviridiae including, but not limited to, human, bovine, ovine, equine, canine, porcine, murine, and simian adenovirus subgenera. In particular, human adenoviruses includes the A-F subgenera as well as the individual serotypes thereof, the individual serotypes and A-F subgenera including but not limited to human adenovirus types 1, 2, 3, 4, 4a, 5, 6, 7, 8, 9, 10, 11 (Ad11a and Ad11p), 12, 13, 14, 15, 16, 17, 18, 19, 19a, 20, 21, 22, 23, 24, 25, 26, 27, 28, 29, 30, 31, 32, 33, 34, 34a, 35, 35p, 36, 37, 38, 39, 40, 41, 42, 43, 44, 45, 46, 47, 48, and 91. Preferred are recombinant viruses derived from human adenovirus types 2 and 5. Unless stated otherwise, all adenovirus type 5 nucleotide numbers are relative to the NCBI reference sequence AC_000008.1, which is depicted herein in SEQ ID NO: 23.

The adenovirus replication cycle has two phases: an early phase, during which 4 transcription units E1, E2, E3, and E4 are expressed, and a late phase which occurs after the onset of viral DNA synthesis when late transcripts are expressed primarily from the major late promoter (MLP). The late messages encode most of the virus's structural proteins. The gene products of E1, E2 and E4 are responsible for transcriptional activation, cell transformation, viral DNA replication, as well as other viral functions, and are necessary for viral growth.

The term “operably linked” refers to a linkage of polynucleotide elements in a functional relationship. A nucleic acid sequence is “operably linked” when it is placed into a functional relationship with another nucleic acid sequence. For instance, a promoter or enhancer is operably linked to a gene if it affects the transcription of the gene. Operably linked nucleotide sequences are typically contiguous. However, as enhancers generally function when separated from the promoter by several kilobases and intronic sequences may be of variable lengths, some polynucleotide elements may be operably linked but not directly flanked and may even function in trans from a different allele or chromosome.

In certain embodiments, the virus has one or more modifications to a regulatory, sequence or promoter. A modification to a regulatory sequence or promoter comprises a deletion, substitution, or addition of one or more nucleotides compared to the wild-type sequence of the regulatory sequence or promoter.

In certain embodiments, the modification of a regulatory sequence or promoter comprises a modification of sequence of a transcription factor binding site to reduce affinity for the transcription factor, for example, by deleting a portion thereof, or by inserting a single point mutation into the binding site. In certain embodiments, the additional modified regulatory sequence enhances expression in neoplastic cells, but attenuates expression in normal cells.

In certain embodiments, the modified regulatory sequence is operably linked to a sequence encoding a protein. In certain embodiments, at least one of the adenoviral E1a and E1b genes (coding regions) is operably linked to a modified regulatory sequence. In certain embodiments, the E1a gene is operably linked to the modified regulatory sequence.

The E1a regulatory sequence contains five binding sites for the transcription factor Pea3, designated Pea3 I, Pea3 II, Pea3 III, Pea3 IV, and Pea3 V, where Pea3 I is the Pea3 binding site most proximal to the E1a start site, and Pea3 V is most distal. The E1a regulatory sequence also contains binding sites for the transcription factor E2F, hereby designated E2F I and E2F II, where E2F I is the E2F binding site most proximal to the E1a start site, and E2F II is more distal. From the E1a start site, the binding sites are arranged: Pea3 I, E2F I, Pea3 II, E2F II, Pea3 III, Pea3 IV, and Pea3 V.

In certain embodiments, at least one of these seven binding sites, or a functional portion thereof, is deleted. A “functional portion” is a portion of the binding site that, when deleted, decreases or even eliminates the functionality, e.g. binding affinity, of the binding site to its respective transcription factor (Pea3 or E2F) by, for example, at least 40%, 50%, 60%, 70%, 80%, 90%, 95% or 100% relative to the complete sequence. In certain embodiments, one or more entire binding sites are deleted. In certain embodiments, a functional portion of one or more binding sites is deleted. A “deleted binding site” encompasses both the deletion of an entire binding site and the deletion of a functional portion. When two or more binding sites are deleted, any combination of entire binding site deletion and functional portion deletion may be used.

In certain embodiments, at least one Pea3 binding site, or a functional portion thereof, is deleted. The deleted Pea3 binding site can be Pea3 I, Pea3 II, Pea3 III, Pea3 IV, and/or Pea3 V. In certain embodiments, the deleted Pea3 binding site is Pea3 II, Pea3 III, Pea3 IV, and/or Pea3 V. In certain embodiments, the deleted Pea3 binding site is Pea3 IV and/or Pea3 V. In certain embodiments, the deleted Pea3 binding site is Pea3 II and/or Pea3 III. In certain embodiments, the deleted Pea3 binding site is both Pea3 II and Pea3 III. In certain embodiments, the Pea3 I binding site, or a functional portion thereof, is retained.

In certain embodiments, at least one E2F binding site, or a functional portion thereof, is deleted. In certain embodiments, at least one E2F binding site, or a functional portion thereof, is retained. In certain embodiments, the retained E2F binding site is E2F I and/or E2F II. In certain embodiments, the retained E2F binding site is E2F II. In certain embodiments the total deletion consists essentially of one or more of Pea3 II, Pea3 III, Pea3 IV, and/or Pea3 V, or functional portions thereof. In certain embodiments, the virus has a deletion of a 50 base pair region located from −304 to −255 upstream of the E1a initiation site, e.g., corresponding to 195-244 of the Ad5 genome (SEQ ID NO: 23), hereafter referred to as the TAV-255 deletion. In certain embodiments, the TAV-255 deletion results in an E1a promoter that comprises the sequence GGTGTTTTGG (SEQ ID NO: 28).

The adenoviral E1b-19k gene functions primarily as an anti-apoptotic gene and is a homolog of the cellular anti-apoptotic gene, BCL-2. Since host cell death prior to maturation of the progeny viral particles would restrict viral replication, E1b-19k is expressed as part of the El cassette to prevent premature cell death thereby allowing the infection to proceed and yield mature virions. Accordingly, in certain embodiments, a recombinant adenovirus is provided that includes an E1b-19K insertion site, e.g., the adenovirus has a nucleotide sequence encoding a therapeutic transgene inserted into an E1b-19K insertion site. In certain embodiments, the adenovirus comprises a nucleotide sequence encoding a therapeutic transgene inserted into an E1b-19K insertion site, wherein the insertion site is located between the start site of E1b-19K (i.e., the nucleotide sequence encoding the start codon of E1b-19k, e.g., corresponding to nucleotides 1714-1716 of SEQ ID NO: 23) and the start site of E1b-55K (i.e., the nucleotide sequence encoding the start codon of E1b-55k, e.g., corresponding to nucleotides 2019-2021 of SEQ ID NO: 23).

### II. Methods of Viral Production

Methods for producing recombinant viruses of the invention are known in the art. Typically, a disclosed virus is produced in a suitable host cell line using conventional techniques including culturing a transfected or infected host cell under suitable conditions so as to allow the production of infectious viral particles. Nucleic acids encoding viral genes can be incorporated into plasmids and introduced into host cells through conventional transfection or transformation techniques. Exemplary suitable host cells for production of disclosed viruses include human cell lines such as HeLa, Hela-S3, HEK293, 911, A549, HER96, or PER-C6 cells. Specific production and purification conditions will vary depending upon the virus and the production system employed. For adenovirus, the traditional method for the generation of viral particles is co-transfection followed by subsequent in vivo recombination of a shuttle plasmid (usually containing a small subset of the adenoviral genome and optionally containing a potential transgene an expression cassette) and an adenoviral helper plasmid (containing most of the entire adenoviral genome).

Alternative technologies for the generation of adenovirus include utilization of the bacterial artificial chromosome (BAC) system, in vivo bacterial recombination in a recA÷bacterial strain utilizing two plasmids containing complementary adenoviral sequences, and the yeast artificial chromosome (YAC) system.

Following production, infectious viral particles are recovered from the culture and optionally purified. Typical purification steps may include plaque purification, centrifugation, e.g., cesium chloride gradient centrifugation, clarification, enzymatic treatment, e.g., benzonase or protease treatment, chromatographic steps, e.g., ion exchange chromatography or filtration steps.

### III. Therapeutic Transgenes

A disclosed recombinant adenovirus may comprise a nucleotide sequence that encodes for a therapeutic transgene. In certain embodiments, a disclosed recombinant comprise virus may comprise a first nucleotide sequence and a second nucleotide sequence that encode for a first and a second therapeutic transgene, respectively. In certain embodiments, a disclosed recombinant comprise virus may comprise a first nucleotide sequence, a second nucleotide sequence, and a third nucleotide sequence that encode for a first, second, and third therapeutic transgene, respectively.

A therapeutic transgene may encode a therapeutic nucleic acid, e.g., an antisense RNA or ribozyme RNA. The therapeutic transgene may encode a therapeutic peptide or polypeptide, e.g., an oncoprotein, tumor suppressor peptide or polypeptide, enzyme, cytokine, immune modulating peptide or polypeptide, antibody, lytic peptide, vaccine antigen, a peptide or polypeptide which complements genetic defects in somatic cells, or a peptide or polypeptide which catalyzes processes leading to cell death.

In certain embodiments, in any of the foregoing viruses, the first and/or second therapeutic transgene, the first, second, and/or third therapeutic transgenes, or any of the therapeutic transgenes encode a therapeutic polypeptide selected from the group consisting of CD80, CD137L, IL-23A/p19, p40, endostatin, angiostatin, ICAM-1, and a TGF-β trap.

In certain embodiments, in any of the foregoing viruses, the first and/or second therapeutic transgene, the first, second, and/or third therapeutic transgenes, or any of the therapeutic transgenes encode a therapeutic polypeptide selected from the group consisting of CD80, CD137L, IL-23, IL-23A/p19, p40, IL-27, IL-27A/p28, IL-27B/EBI3, endostatin, angiostatin, ICAM-1, a TGF-β trap, TGF-β, CD19, CD20, IL-1, IL-3, IL-4, IL-5, IL-6, IL-8, IL-9, CD154, CD86, BORIS/CTCFL, FGF, IL-24, MAGE, NY-ESO-1, acetylcholine, interferon-gamma, DKK1/Wnt, p53, thymidine kinase, an anti-PD-1 antibody heavy chain or light chain, and an anti-PD-L1 antibody heavy chain or light chain.

In certain embodiments, the first therapeutic transgene encodes CD80, and/or the second therapeutic transgene encodes CD137L. In further embodiments, the first therapeutic transgene encodes CD137L, and/or the second therapeutic transgene encodes CD80. CD80 is a costimulatory molecule that can play a role in activating naive CD8+ T cells. CD8+ T cells are activated when the T cell receptor (TCR) binds to a class I major histocompatibility complex (MHC) on an antigen presenting cell (APC) presenting a peptide that the TCR recognizes. In addition to the TCR-MHC interaction, the T cell must also receive a costimulatory signal through a CD28 molecule on the T cell binding to either CD80 or CD86 on the APC. The T cell can then become activated, dividing and gaining the ability to mount a response against other cells that display the same peptide. Activation also leads to expression of other molecules including CTLA-4 and CD137 on the T cell. CTLA-4 binds to CD80 with higher affinity than CD28, and CTLA-4 binding to CD80 leads to inactivation of the T cell. CD137 binds to CD137L, and upon binding it further activates the T cell and promotes cell division and persistence of an immune response.

In certain embodiments the first and/or second therapeutic transgenes are selected from the group consisting of CD80 and CD137L, e.g., the first therapeutic transgene encodes CD80 and the second therapeutic transgene encodes CD137L. An exemplary nucleotide sequence encoding human CD80 is depicted in SEQ ID NO: 5, and an exemplary nucleotide sequence encoding human CD137L is depicted in SEQ ID NO: 7. In certain embodiments, the recombinant adenovirus comprises a nucleotide sequence encoding an amino acid sequence that is encoded by SEQ ID NO: 5, and/or SEQ ID NO: 7, or a sequence having 80%, 85%, 86%, 87%, 88%, 89%, 90%, 91%, 92%, 93%, 94%, 95%, 96%, 97%, 98%, or 99% sequence identity to SEQ ID NO: 5, and/or SEQ ID NO: 7. In certain embodiments, the recombinant adenovirus comprises the nucleotide sequence of SEQ ID NO: 6, and/or SEQ ID NO: 8, or a sequence having 80%, 85%, 86%, 87%, 88%, 89%, 90%, 91%, 92%, 93%, 94%, 95%, 96%, 97%, 98%, or 99% sequence identity to SEQ ID NO: 6, and/or SEQ ID NO: 8. In certain embodiments, the recombinant adenovirus comprises the nucleotide sequence of SEQ ID NO: 27, or a sequence having 80%, 85%, 86%, 87%, 88%, 89%, 90%, 91%, 92%, 93%, 94%, 95%, 96%, 97%, 98%, or 99% sequence identity to SEQ ID NO: 27.

In certain embodiments, in any of the foregoing viruses, the first, second, and/or third therapeutic transgenes are selected from the group consisting of CD80, CD137L, and ICAM-1, e.g., the first therapeutic transgene encodes CD80, the second therapeutic transgene encodes CD137L, and the third therapeutic transgene encodes ICAM-1. An exemplary nucleotide sequence encoding human ICAM1 is depicted in SEQ ID NO: 32. In certain embodiments, the recombinant adenovirus comprises a nucleotide sequence encoding an amino acid sequence that is encoded by SEQ ID NO: 5, SEQ ID NO: 7, and/or SEQ ID NO: 32, or a sequence having 80%, 85%, 86%, 87%, 88%, 89%, 90%, 91%, 92%, 93%, 94%, 95%, 96%, 97%, 98%, or 99% sequence identity to SEQ ID NO: 5, SEQ ID NO: 7, and/or SEQ ID NO: 32. In certain embodiments, the recombinant adenovirus comprises the nucleotide sequence of SEQ ID NO: 31, SEQ ID NO: 9, or SEQ ID NO: 22, or a sequence having 80%, 85%, 86%, 87%, 88%, 89%, 90%, 91%, 92%, 93%, 94%, 95%, 96%, 97%, 98%, or 99% sequence identity to SEQ ID NO: 31, SEQ ID NO: 9, or SEQ ID NO: 22.

In certain embodiments, the first and second therapeutic transgene encode a first and second subunit, respectively, of a heterodimeric cytokine. For example, in certain embodiments the first and/or second therapeutic transgenes are selected from the group consisting of IL-23A/p19 and p40, which make up the heterodimeric cytokine IL-23. For example, the first therapeutic transgene may encode IL-23A/p19 and the second therapeutic transgene may encode p40. An exemplary nucleotide sequence encoding human IL-23A/p19 is depicted in SEQ ID NO: 12, and an exemplary nucleotide sequence encoding human p40 is depicted in SEQ ID NO: 10. In certain embodiments, the recombinant adenovirus comprises a nucleotide sequence encoding an amino acid sequence that is encoded by SEQ ID NO: 12 and/or SEQ ID NO: 10, or a sequence having 80%, 85%, 86%, 87%, 88%, 89%, 90%, 91%, 92%, 93%, 94%, 95%, 96%, 97%, 98%, or 99% sequence identity to SEQ ID NO: 12 and/or SEQ ID NO: 10. In certain embodiments, the recombinant adenovirus comprises the nucleotide sequence of SEQ ID NO: 13, or a sequence having 80%, 85%, 86%, 87%, 88%, 89%, 90%, 91%, 92%, 93%, 94%, 95%, 96%, 97%, 98%, or 99% sequence identity to SEQ ID NO: 13.

Additionally, in certain embodiments, the first and/or second therapeutic transgenes are selected from the group consisting of IL-27A/p28 and IL-27B/EBI3, which make up the heterodimeric cytokine IL-27. For example, the first therapeutic transgene may encode IL-IL-27A/p28 and the second therapeutic transgene may encode IL-27B/EBI3.

When tumors grow beyond approximately 2 mm³ in diameter, they require the proliferation of an independent network of blood vessels to supply nutrients and oxygen and remove waste products. This new vessel formation, i.e., neovascularization, is known as tumor angiogenesis. Pro-angiogenic factors include vascular endothelial growth factor (VEGF), basic fibroblast growth factor (bFGF), platelet-derived growth factor (PDGF), epidermal growth factor (EGF), interleukin 8 (IL-8), and the angiopoietins. Endostatin and angiostatin are naturally occurring anti-angiogenic proteins that are reported to inhibit neovascularization.

In certain embodiments, the first and/or second therapeutic transgenes are selected from the group consisting of endostatin and angiostatin. In certain embodiments, the first therapeutic transgene is endostatin and the second therapeutic transgene is angiostatin. In certain embodiments, the first therapeutic transgene is angiostatin and the second therapeutic transgene is endostatin.

Endostatin is a proteolytic fragment of collagen XVIII. An exemplary human collagen XVIII amino acid sequence, corresponding to NCBI Reference Sequence NP_085059.2, is depicted in SEQ ID NO: 33. Endostatin can result from proteolytic cleavage of collagen XVIII at different sites. The non-collagenous 1 (NC1) domain at the C-terminus of collagen XVIII is generally considered responsible for the anti-angiogenic effects of endostatin. An exemplary human collagen XVIII NC1 domain amino acid sequence is depicted in SEQ ID NO: 37. Accordingly, as used herein, the term “endostatin” is understood to mean a protein comprising the amino acid sequence of SEQ ID NO: 37, or comprising an amino acid sequence having greater than 80%, 85%, 86%, 87%, 88%, 89%, 90%, 91%, 92%, 93%, 94%, 95%, 96%, 97%, 98%, or 99% sequence identity to SEQ ID NO: 37, or a fragment of any of the forgoing that is capable of noncovalently oligomerizing into trimers, for example, through an association domain present in SEQ ID NO: 37. Oligomerization can be assayed by any method known in the art, including, for example, size exclusion chromatography, analytical ultracentrifugation, scattering techniques, NMR spectroscopy, isothermal titration calorimetry, fluorescence anisotropy and mass spectrometry.

In certain embodiments, a disclosed recombinant virus comprises a nucleotide sequence encoding the amino acid sequence of SEQ ID NO: 37 or SEQ ID NO: 38, or a sequence having 80%, 85%, 86%, 87%, 88%, 89%, 90%, 91%, 92%, 93%, 94%, 95%, 96%, 97%, 98%, or 99% sequence identity to SEQ ID NO: 37 or SEQ ID NO: 38.

Angiostatin is a proteolytic fragment of plasminogen. An exemplary human plasminogen amino acid sequence, corresponding to NCBI Reference Sequence NP_000292.1, is depicted in SEQ ID NO: 34.

Angiostatin can result from proteolytic cleavage of plasminogen at different sites. Plasminogen has five kringle domains, which are generally considered responsible for the anti-angiogenic effects of angiostatin. An exemplary amino acid sequence of the first kringle domain of human plasminogen is depicted in SEQ ID NO: 39, an exemplary amino acid sequence of the second kringle domain of human plasminogen is depicted in SEQ ID NO: 40, an exemplary amino acid sequence of the third kringle domain of human plasminogen is depicted in SEQ ID NO: 41, an exemplary amino acid sequence of the fourth kringle domain of human plasminogen is depicted in SEQ ID NO: 42, and an exemplary amino acid sequence of the fifth kringle domain of human plasminogen is depicted in SEQ ID NO: 43. Accordingly, as used herein, the term “angiostatin” is understood to mean a protein comprising the amino acid sequence of SEQ ID NO: 39, SEQ ID NO: 40, SEQ ID NO: 41, SEQ ID NO: 42, or SEQ ID NO: 43, or comprising an amino acid sequence having greater than 80%, 85%, 86%, 87%, 88%, 89%, 90%, 91%, 92%, 93%, 94%, 95%, 96%, 97%, 98%, or 99% sequence identity to SEQ ID NO: 39, SEQ ID NO: 40, SEQ ID NO: 41, SEQ ID NO: 42, or SEQ ID NO: 43, or a fragment of any of the foregoing that is capable of antagonizing endothelial cell migration and/or endothelial cell proliferation. Endothelial cell migration and/or proliferation can be assayed by any method known in the art, including, for example, those described in Guo et al. (2014) METHODS MOL. BIOL. 1135: 393-402.

In certain embodiments, a disclosed recombinant virus comprises a nucleotide sequence encoding the amino acid sequence of SEQ ID NO: 39, SEQ ID NO: 40, SEQ ID NO: 41, SEQ ID NO: 42, SEQ ID NO: 43, or SEQ ID NO: 44 or a sequence having 80%, 85%, 86%, 87%, 88%, 89%, 90%, 91%, 92%, 93%, 94%, 95%, 96%, 97%, 98%, or 99% sequence identity to SEQ ID NO: 39, SEQ ID NO: 40, SEQ ID NO: 41, SEQ ID NO: 42, SEQ ID NO: 43, or SEQ ID NO: 44.

In certain embodiments, a disclosed recombinant virus comprises the nucleotide sequence of SEQ ID NO: 11, or a sequence having 80%, 85%, 86%, 87%, 88%, 89%, 90%, 91%, 92%, 93%, 94%, 95%, 96%, 97%, 98%, or 99% sequence identity to SEQ ID NO: 11.

### IV. Methods of Treatment

For therapeutic use, a recombinant adenovirus is preferably is combined with a pharmaceutically acceptable carrier. As used herein, “pharmaceutically acceptable carrier” means buffers, carriers, and excipients suitable for use in contact with the tissues of human beings and animals without excessive toxicity, irritation, allergic response, or other problem or complication, commensurate with a reasonable benefit/risk ratio. The carrier(s) should be “acceptable” in the sense of being compatible with the other ingredients of the formulations and not deleterious to the recipient. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art.

Pharmaceutical compositions containing recombinant adenoviruses disclosed herein can be presented in a dosage unit form and can be prepared by any suitable method. A pharmaceutical composition should be formulated to be compatible with its intended route of administration. Examples of routes of administration are intravenous (IV), intradermal, inhalation, transdermal, topical, transmucosal, and rectal administration. A preferred route of administration for fusion proteins is IV infusion. Useful formulations can be prepared by methods known in the pharmaceutical art. For example, see Remington's Pharmaceutical Sciences, 18th ed. (Mack Publishing Company, 1990). Formulation components suitable for parenteral administration include a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor EL™ (BASF, Parsippany, N.J.) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyethylene glycol), and suitable mixtures thereof.

Pharmaceutical formulations preferably are sterile. Sterilization can be accomplished by any suitable method, e.g., filtration through sterile filtration membranes. Where the composition is lyophilized, filter sterilization can be conducted prior to or following lyophilization and reconstitution.

The term “effective amount” as used herein refers to the amount of an active component (e.g., the amount of a recombinant adenovirus of the present invention) sufficient to effect beneficial or desired results. An effective amount can be administered in one or more administrations, applications or dosages and is not intended to be limited to a particular formulation or administration route.

In certain embodiments, a therapeutically effective amount of active component is in the range of 0.1 mg/kg to 100 mg/kg, e.g., 1 mg/kg to 100 mg/kg, 1 mg/kg to 10 mg/kg. In certain embodiments, a therapeutically effective amount of the recombinant adenovirus is in the range of 10² to 10¹⁵ plaque forming units (pfus), e.g., 10² to 10¹⁰, 10² to 10⁵, 10⁵ to 10¹⁵, 10⁵ to 10¹⁰, or 10¹⁰ to 10¹⁵ plaque forming units. The amount administered will depend on variables such as the type and extent of disease or indication to be treated, the overall health of the patient, the in vivo potency of the antibody, the pharmaceutical formulation, and the route of administration. The initial dosage can be increased beyond the upper level in order to rapidly achieve the desired blood-level or tissue-level. Alternatively, the initial dosage can be smaller than the optimum, and the daily dosage may be progressively increased during the course of treatment. Human dosage can be optimized, e.g., in a conventional Phase I dose escalation study designed to run from 0.5 mg/kg to 20 mg/kg. Dosing frequency can vary, depending on factors such as route of administration, dosage amount, serum half-life of the antibody, and the disease being treated. Exemplary dosing frequencies are once per day, once per week and once every two weeks. A preferred route of administration is parenteral, e.g., intravenous infusion. Formulation of monoclonal antibody-based drugs is within ordinary skill in the art. In certain embodiments, a recombinant adenovirus is lyophilized, and then reconstituted in buffered saline, at the time of administration.

The recombinant adenoviruses disclosed herein can be used to treat various medical indications. For example, the recombinant adenoviruses can be used to treat cancers. The cancer cells are exposed to a therapeutically effective amount of the recombinant adenovirus so as to inhibit or reduce proliferation of the cancer cells. The invention provides a method of treating a cancer in a subject. The method comprises administering to the subject an effective amount of a recombinant adenovirus of the invention either alone or in a combination with another therapeutic agent to treat the cancer in the subject. In certain embodiments, administering an effective amount of a recombinant adenovirus to a subject reduces tumor load in that subject by at least 30%, at least 40%, at least 50%, at least 60%, at least 70%, at least 80%, or at least 90%.

As used herein, “treat”, “treating” and “treatment” mean the treatment of a disease in a subject, e.g., in a human. This includes: (a) inhibiting the disease, i.e., arresting its development; and (b) relieving the disease, i.e., causing regression of the disease state. As used herein, the terms “subject” and “patient” refer to an organism to be treated by the methods and compositions described herein. Such organisms preferably include, but are not limited to, mammals (e.g., murines, simians, equines, bovines, porcines, canines, felines, and the like), and more preferably includes humans.

Examples of cancers include solid tumors, soft tissue tumors, hematopoietic tumors and metastatic lesions. Examples of hematopoietic tumors include, leukemia, acute leukemia, acute lymphoblastic leukemia (ALL), B-cell, T-cell or FAB ALL, acute myeloid leukemia (AML), chronic myelocytic leukemia (CML), chronic lymphocytic leukemia (CLL), e.g., transformed CLL, diffuse large B-cell lymphomas (DLBCL), follicular lymphoma, hairy cell leukemia, myelodysplastic syndrome (MDS), a lymphoma, Hodgkin's disease, a malignant lymphoma, non-Hodgkin's lymphoma, Burkitt's lymphoma, multiple myeloma, or Richter's Syndrome (Richter's Transformation). Examples of solid tumors include malignancies, e.g., sarcomas, adenocarcinomas, and carcinomas, of the various organ systems, such as those affecting head and neck (including pharynx), thyroid, lung (small cell or non-small cell lung carcinoma (NSCLC)), breast, lymphoid, gastrointestinal (e.g., oral, esophageal, stomach, liver, pancreas, small intestine, colon and rectum, anal canal), genitals and genitourinary tract (e.g., renal, urothelial, bladder, ovarian, uterine, cervical, endometrial, prostate, testicular), CNS (e.g., neural or glial cells, e.g., neuroblastoma or glioma), or skin (e.g., melanoma).

In certain embodiments, the cancer is selected from the group consisting of melanoma, squamous cell carcinoma of the skin, basal cell carcinoma, head and neck cancer, breast cancer, anal cancer, cervical cancer, non-small cell lung cancer, mesothelioma, small cell lung cancer, renal cell carcinoma, prostate cancer, gastroesophageal cancer, colorectal cancer, testicular cancer, bladder cancer, ovarian cancer, hepatocellular carcinoma, cholangiocarcinoma, brain cancer, endometrial cancer, neuroendocrine cancer, and pancreatic cancer.

In certain embodiments, a recombinant adenovirus is administered to the subject in combination with one or more therapies, e.g., surgery, radiation, chemotherapy, immunotherapy, hormone therapy, or virotherapy.

In certain embodiments, a recombinant adenovirus of the invention is administered in combination with a tyrosine kinase inhibitor, e.g., erlotinib.

In certain embodiments, a recombinant adenovirus of the invention is administered in combination with a checkpoint inhibitor, e.g., an anti-CTLA-4 antibody, an anti-PD-1 antibody, or an anti-PD-L1 antibody. Exemplary anti-PD-1 antibodies include, for example, nivolumab (Opdivo®, Bristol-Myers Squibb Co.), pembrolizumab (Keytruda®, Merck Sharp & Dohme Corp.), PDR001 (Novartis Pharmaceuticals), and pidilizumab (CT-011, Cure Tech). Exemplary anti-PD-L1 antibodies include, for example, atezolizumab (Tecentriq®, Genentech), duvalumab (AstraZeneca), MEDI4736, avelumab, and BMS 936559 (Bristol Myers Squibb Co.).

The term administered “in combination,” as used herein, is understood to mean that two (or more) different treatments are delivered to the subject during the course of the subject's affliction with the disorder, such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In certain embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In certain embodiments, the effective amount of the recombinant virus is identified by measuring an immune response to an antigen in the subject and/or the method of treating the subject further comprises measuring an immune response to an antigen in the subject. Hyperproliferative diseases, e.g., cancers, may be characterized by immunosuppression, and measuring an immune response to an antigen in the subject may be indicative of the level of immunosuppression in the subject. Accordingly, measuring an immune response to an antigen in the subject may be indicative of the efficacy of the treatment and/or the effective amount of the recombinant virus. The immune response to the antigen in the subject may be measured by any method known in the art. In certain embodiments, the immune response to the antigen is measured by injecting the subject with the antigen at an injection site on the skin of the subject and measuring the size of an induration or amount of inflammation at the injection site. In certain embodiments, the immune response to the antigen is measured by release of a cytokine from a cell of the subject (e.g., interferon gamma, IL-4 and/or IL-5) upon exposure to the antigen.

Throughout the description, where viruses, compositions and systems are described as having, including, or comprising specific components, or where processes and methods are described as having, including, or comprising specific steps, it is contemplated that, additionally, there are compositions, devices, and systems of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a virus, a composition, a system, a method, or a process described herein can be combined in a variety of ways without departing from the spirit and scope of the present invention, whether explicit or implicit herein. For example, where reference is made to a particular virus, that virus can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

At various places in the present specification, viruses, compositions, systems, processes and methods, or features thereof, are disclosed in groups or in ranges. It is specifically intended that the description include each and every individual subcombination of the members of such groups and ranges. By way of other examples, an integer in the range of 1 to 20 is specifically intended to individually disclose 1, 2, 3, 4, 5, 6, 7, 8, 9, 10, 11, 12, 13, 14, 15, 16, 17, 18, 19, and 20.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

### EXAMPLES

The following Examples are merely illustrative and are not intended to limit the scope or content of the invention in any way.

### Example 1: Construction of a CD80 and CD137L Expressing Adenovirus

This Example describes the production of a recombinant adenovirus type 5 (Ad5) that expresses the murine forms of CD80 and CD137L.

An adenovirus type 5 virus was constructed that carried the deletion of a nucleotide region located from −304 to −255 upstream of the E1a initiation, which renders E1a expression cancer-selective (as previously described in U.S. Pat. No. 9,073,980). The resulting virus is hereafter referred to as TAV.

TAV was further modified to carry a SalI site at the start site of the E1b-19k region and an XhoI site 200 base pairs 3′ of the SalI site to facilitate insertion of therapeutic transgenes. The nucleotide sequence of the modified E1b-19k region is as follows, with the residual bases from the fused SalI and XhoI sites underlined:

(SEQ ID NO: 15)
ATCTTGGTTACATCTGACCTCGTCGAGTCACCAGGCGCTTTTCCAA.

TAV was further modified to carry the dl309 disruption of the E3 region's RIDα, RIDβ, and 14.7k genes The nucleotide sequence of the modified E3 region is as follows, with the hyphen indicating the point of deletion:

(SEQ ID NO: 16)
TCTTTTCTCTTACAGTATGA-TAATAAAAAAAAATAATAAAGCATCACTT
AC.

The resulting virus, including both the modified E1b-19k region and the modified E3 region is hereafter referred to as TAV-Δ19k.

Where indicated, murine CD80 (mCD80) or human CD80 (hCD80) was cloned into the modified E1b-19k region.

The sequence of mCD80 in the modified E1b-19k region is as follows, with the coding region in lower case, and the flanking adenoviral sequences including the SalI and XhoI sites capitalized:

(SEQ ID NO: 17)
ATCTGACCTCGTCGACatggcttgcaattgtcagttgatgcaggatacac
cactcctcaagtttccatgtccaaggctcattcttctctttgtgctgctg
attcgtctttcacaagtgtcttcagatgttgatgaacaactgtccaagtc
agtgaaagataaggtattgctgccttgccgttacaactctcctcatgaag
atgagtctgaagaccgaatctactggcaaaaacatgacaaagtggtgctg
tctgtcattgctgggaaactaaaagtgtggcccgagtataagaaccggac
tttatatgacaacactacctactctcttatcatcctgggcctggtccttt
cagaccggggcacatacagctgtgtcgttcaaaagaaggaaagaggaacg
tatgaagttaaacacttggctttagtaaagttgtccatcaaagctgactt
ctctacccccaacataactgagtctggaaacccatctgcagacactaaaa
ggattacctgctttgcttccgggggtttcccaaagcctcgcttctcttgg
ttggaaaatggaagagaattacctggcatcaatacgacaatttcccagga
tcctgaatctgaattgtacaccattagtagccaactagatttcaatacga
ctcgcaaccacaccattaagtgtctcattaaatatggagatgctcacgtg
tcagaggacttcacctgggaaaaacccccagaagaccctcctgatagcaa
gaacacacttgtgctctttggggcaggattcggcgcagtaataacagtcg
tcgtcatcgttgtcatcatcaaatgcttctgtaagcacagaagctgtttc
agaagaaatgaggcaagcagagaaacaaacaacagccttaccttcgggcc
tgaagaagcattagctgaacagaccgtcttcctttagCTCGAGTCACCAG
GCG.

The sequence of hCD80 in the modified E1b-19k region is as follows, with the coding region in lower case, and the flanking adenoviral sequences including the SalI and XhoI sites capitalized:

(SEQ ID NO: 18)
GCGCCGTGGGCTAATCTTGGTTACATCTGACCTCGTCGACatgggccaca
cacggaggcagggaacatcaccatccaagtgtccatacctcaatttcttt
cagctcttggtgctggctggtctttctcacttctgttcaggtgttatcca
cgtgaccaaggaagtgaaagaagtggcaacgctgtcctgtggtcacaatg
tttctgttgaagagctggcacaaactcgcatctactggcaaaaggagaag
aaaatggtgctgactatgatgtctggggacatgaatatatggcccgagta
caagaaccggaccatctttgatatcactaataacctctccattgtgatcc
tggctctgcgcccatctgacgagggcacatacgagtgtgttgttctgaag
tatgaaaaagacgctttcaagcgggaacacctggctgaagtgacgttatc
agtcaaagctgacttccctacacctagtatatctgactttgaaattccaa
cttctaatattagaaggataatttgctcaacctctggaggttttccagag
cctcacctctcctggttggaaaatggagaagaattaaatgccatcaacac
aacagtttcccaagatcctgaaactgagctctatgctgttagcagcaaac
tggatttcaatatgacaaccaaccacagcttcatgtgtctcatcaagtat
ggacatttaagagtgaatcagaccttcaactggaatacaaccaagcaaga
gcattttcctgataacctgctcccatcctgggccattaccttaatctcag
taaatggaatttttgtgatatgctgcctgacctactgctttgccccaaga
tgcagagagagaaggaggaatgagagattgagaagggaaagtgtacgccc
tgtataaCTCGAGTCACCAGGCGCTTTTCCAAGAGAAGGTCATCAAG.

Where indicated murine CD137L (mCD137L) or human CD137L (hCD137L) were cloned into the modified E3 region.

The sequence of mCD137L in the modified E3 region is as follows, with the coding region in lower case, and the flanking adenoviral sequences capitalized:

(SEQ ID NO: 19)
ATGTTCTTTTCTCTTACAGTATGATTAAATGAGACatggaccagcacaca
cttgatgtggaggataccgcggatgccagacatccagcaggtacttcgtg
cccctcggatgcggcgctcctcagagataccgggctcctcgcggacgctg
cgctcctctcagatactgtgcgccccacaaatgccgcgctccccacggat
gctgcctaccctgcggttaatgttcgggatcgcgaggccgcgtggccgcc
tgcactgaacttctgttcccgccacccaaagctctatggcctagtcgctt
tggttttgctgcttctgatcgccgcctgtgttcctatcttcacccgcacc
gagcctcggccagcgctcacaatcaccacctcgcccaacctgggtacccg
agagaataatgcagaccaggtcacccctgtttcccacattggctgcccca
acactacacaacagggctctcctgtgttcgccaagctactggctaaaaac
caagcatcgttgtgcaatacaactctgaactggcacagccaagatggagc
tgggagctcatacctatctcaaggtctgaggtacgaagaagacaaaaagg
agttggtggtagacagtcccgggctctactacgtatttttggaactgaag
ctcagtccaacattcacaaacacaggccacaaggtgcagggctgggtctc
tcttgttttgcaagcaaagcctcaggtagatgactttgacaacttggccc
tgacagtggaactgttcccttgctccatggagaacaagttagtggaccgt
tcctggagtcaactgttgctcctgaaggctggccaccgcctcagtgtggg
tctgagggcttatctgcatggagcccaggatgcatacagagactgggagc
tgtcttatcccaacaccaccagctttggactctttcttgtgaaacccgac
aacccatgggaatgaGGTCTCAAAGATCTTATTCCCTTTAACTAATAAA.

The sequence of hCD137L in the modified E3 region is as follows, with the coding region in lower case, and the flanking adenoviral sequences capitalized:

(SEQ ID NO: 20)
ATGTTCTTTTCTCTTACAGTATGATTAAATGAGACatggaatacgcctct
gacgcttcactggaccccgaagccccgtggcctcctgcacctcgcgctcg
cgcctgccgcgtactgccttgggccctggtcgcggggctgctgctcctgc
tcctgctcgctgctgcatgcgctgtatttcttgcatgcccatgggctgtg
tctggggctcgcgcatcacctggctccgcggccagcccgagactccgcga
gggtcccgagctttcgcccgacgatcccgccggcctcttggacctgcggc
agggcatgtttgcgcagctggtggcccaaaatgttctgctgatcgatggg
cccctgagctggtacagtgacccaggcctggcaggcgtgtccctgacggg
gggcctgagctacaaagaggacacgaaggagctggtggtggccaaggctg
gagtctactatgtcttctttcaactagagctgcggcgcgtggtggccggc
gagggctcaggctccgtttcacttgcgctgcacctgcagccactgcgctc
tgctgctggggccgccgccctggctttgaccgtggacctgccacccgcct
cctccgaggctcggaactcggccttcggtttccagggccgcttgctgcac
ctgagtgccggccagcgcctgggcgtccatcttcacactgaggccagggc
acgccatgcctggcagcttacccagggcgccacagtcttgggactcttcc
gggtgacccccgaaatcccagccggactcccttcaccgaggtcggaataa
GGTCTCAAAGATCTTATTCCCTTTAACTAATAAA.

Additionally, where indicated, both human CD80 and CD137L were cloned into the modified E1b-19k region, separated by an internal ribosome entry site (IRES). In these instances, the E1b-19k region contained the human CD80 gene including a stop codon, followed by the IRES from encephalomyocarditis virus, followed by the human CD137L gene. Because the insertion of both the CD80 and CD137L genes in the E1b-19k region would make the viral genome size exceed the packaging limits for an adenovirus, this virus still has the RIDα, RIDβ, and 14.7k gene deletion in the E3 region.

The sequence of hCD80 and hCD137L in the modified E1b-19k region, separated by IRES, is as follows, with the coding region in lower case, the flanking adenoviral sequences capitalized, and the central IRES capitalized:

(SEQ ID NO: 21)
GCGCCGTGGGCTAATCTTGGTTACATCTGACCTCGTCGACatgggccaca
cacggaggcagggaacatcaccatccaagtgtccatacctcaatttcttt
cagctcttggtgctggctggtctttctcacttctgttcaggtgttatcca
cgtgaccaaggaagtgaaagaagtggcaacgctgtcctgtggtcacaatg
tttctgttgaagagctggcacaaactcgcatctactggcaaaaggagaag
aaaatggtgctgactatgatgtctggggacatgaatatatggcccgagta
caagaaccggaccatctttgatatcactaataacctctccattgtgatcc
tggctctgcgcccatctgacgagggcacatacgagtgtgttgttctgaag
tatgaaaaagacgctttcaagcgggaacacctggctgaagtgacgttatc
agtcaaagctgacttccctacacctagtatatctgactttgaaattccaa
cttctaatattagaaggataatttgctcaacctctggaggttttccagag
cctcacctctcctggttggaaaatggagaagaattaaatgccatcaacac
aacagtttcccaagatcctgaaactgagctctatgctgttagcagcaaac
tggatttcaatatgacaaccaaccacagcttcatgtgtctcatcaagtat
ggacatttaagagtgaatcagaccttcaactggaatacaaccaagcaaga
gcattttcctgataacctgctcccatcctgggccattaccttaatctcag
taaatggaatttttgtgatatgctgcctgacctactgctttgccccaaga
tgcagagagagaaggaggaatgagagattgagaagggaaagtgtacgccc
tgtataaTAACGTTACTGGCCGAAGCCGCTTGGAATAAGGCCGGTGTGCG
TTTGTCTATATGTTATTTTCCACCATATTGCCGTCTTTTGGCAATGTGAG
GGCCCGGAAACCTGGCCCTGTCTTCTTGACGAGCATTCCTAGGGGTCTTT
CCCCTCTCGCCAAAGGAATGCAAGGTCTGTTGAATGTCGTGAAGGAAGCA
GTTCCTCTGGAAGCTTCTTGAAGACAAACAACGTCTGTAGCGACCCTTTG
CAGGCAGCGGAACCCCCCACCTGGCGACAGGTGCCTCTGCGGCCAAAAGC
CACGTGTATAAGATACACCTGCAAAGGCGGCACAACCCCAGTGCCACGTT
GTGAGTTGGATAGTTGTGGAAAGAGTCAAATGGCTCTCCTCAAGCGTATT
CAACAAGGGGCTGAAGGATGCCCAGAAGGTACCCCATTGTATGGGATCTG
ATCTGGGGCCTCGGTGCACATGCTTTACATGTGTTTAGTCGAGGTTAAAA
AACGTCTAGGCCCCCCGAACCACGGGGACGTGGTTTTCCTTTGAAAAACA
CGATGATAATatggaatacgcctctgacgcttcactggaccccgaagccc
cgtggcctcctgcacctcgcgctcgcgcctgccgcgtactgccttgggcc
ctggtcgcggggctgctgctcctgctcctgctcgctgctgcatgcgctgt
atttcttgcatgcccatgggctgtgtctggggctcgcgcatcacctggct
ccgcggccagcccgagactccgcgagggtcccgagctttcgcccgacgat
cccgccggcctcttggacctgcggcagggcatgtttgcgcagctggtggc
ccaaaatgttctgctgatcgatgggcccctgagctggtacagtgacccag
gcctggcaggcgtgtccctgacggggggcctgagctacaaagaggacacg
aaggagctggtggtggccaaggctggagtctactatgtcttctttcaact
agagctgcggcgcgtggtggccggcgagggctcaggctccgtttcacttg
cgctgcacctgcagccactgcgctctgctgctggggccgccgccctggct
ttgaccgtggacctgccacccgcctcctccgaggctcggaactcggcctt
cggtttccagggccgcttgctgcacctgagtgccggccagcgcctgggcg
tccatcttcacactgaggccagggcacgccatgcctggcagcttacccag
ggcgccacagtcttgggactcttccgggtgacccccgaaatcccagccgg
actcccttcaccgaggtcggaataaCTCGAGTCACCAGGCGCTTTTCCAA
GAGAAGGTCATCAAG.

Details of the viruses tested are shown in TABLE 1.

TABLE 1

| | E1A | E1b-19k | E3 (RIDα, RIDβ, and 14.7k) |
|---|---|---|---|
| Virus | Promoter | Modification | Modification |
| TAV-Δ19k | TAV-255 | Deleted | Disrupted (containing the dl309 |
| | | | sequence) |
| TAV-mCD80 | TAV-255 | Deleted and Replaced | Disrupted (containing the dl309 |
| | | with murine CD80 | sequence) |
| TAV-mCD137L | TAV-255 | Deleted | Deleted and Replaced with murine |
| | | | CD137L |
| TAV-mCD80- | TAV-255 | Deleted and Replaced | Deleted and Replaced with murine |
| 137L | | with murine CD80 | CD137L |
| TAV-hCD80- | TAV-255 | Deleted and Replaced | Deleted and Replaced with human |
| 137L | | with human CD80 | CD137L |
| TAV-hCD80- | TAV-255 | Deleted and Replaced | Deleted |
| IRES-137L | | with human CD80, | |
| | | IRES, and human | |
| | | CD137L | |

### Example 2: CD80 and CD137L Gene Expression

This example describes the expression of CD80 and/or CD137L from the recombinant adenoviruses produced as described in Example 1.

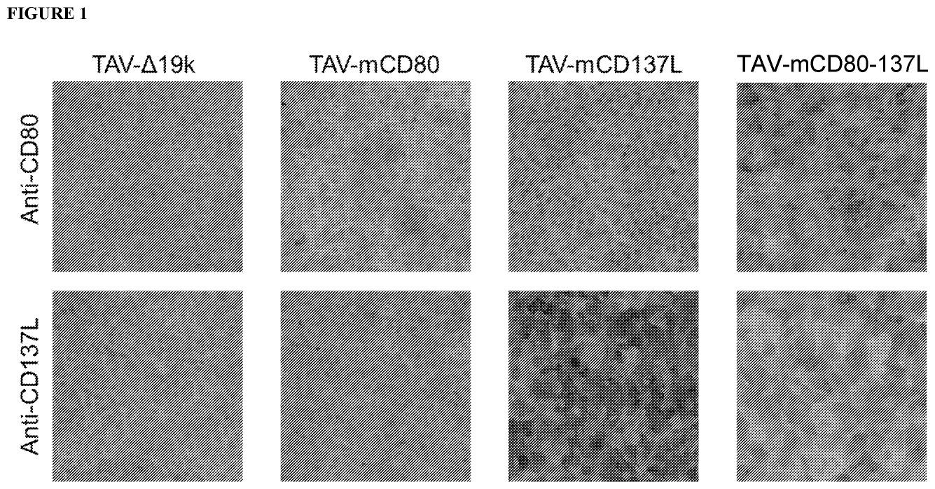

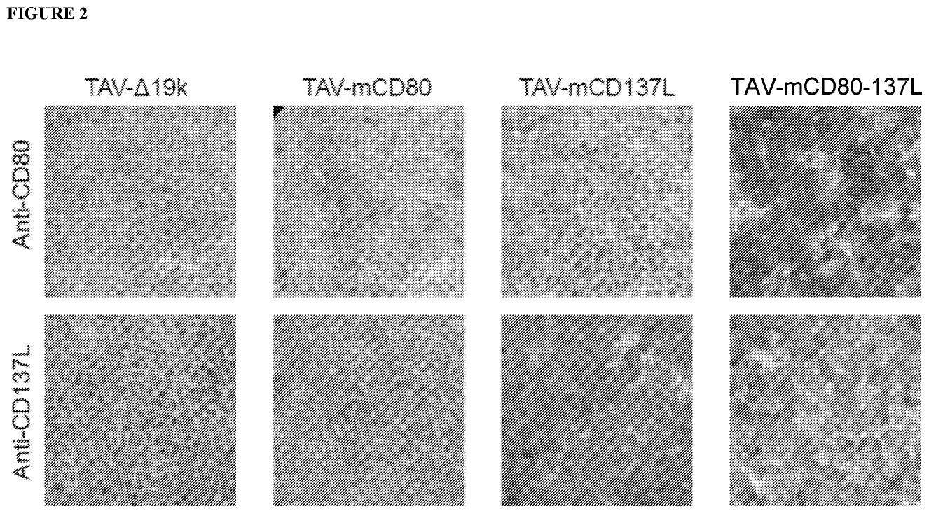

ADS-12 cells (mouse lung adenocarcinoma cells) were infected with the TAV-Δ19k, TAV-mCD80. TAV-mCD137L, and TAV-mCD80-137L viruses, and infected cells were stained for CD80 and CD137L with immunocytochemistry. As depicted in FIG. 1 and FIG. 2, mCD80 was expressed after infection with either TAV-mCD80 or TAV-mCD80-137L, and CD137L was expressed after infection with either TAV-mCD137L or TAV-mCD80-137L. Importantly, both genes were expressed with the TAV-mCD80-137L virus, demonstrating that the single virus drove expression of two therapeutic genes.

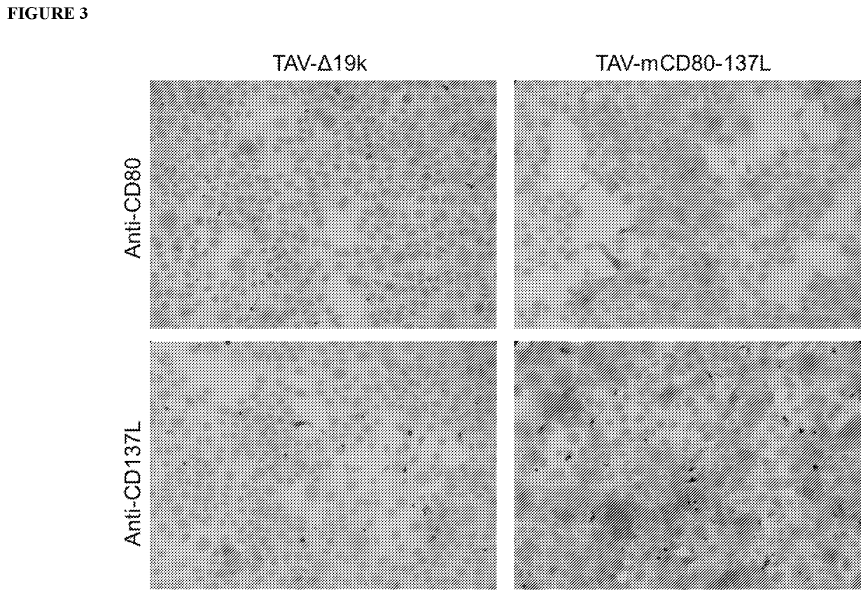

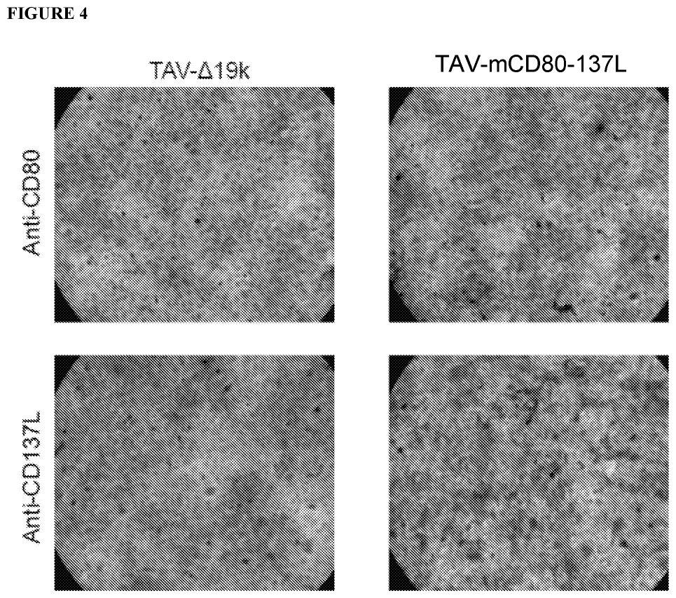

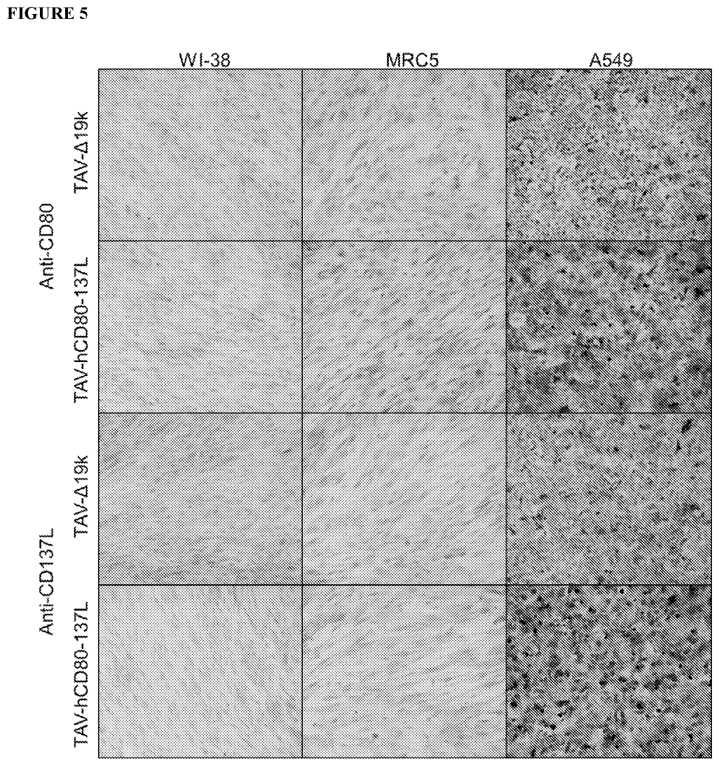

4T1 cells (mouse mammary carcinoma cells) were infected with the TAV-Δ19k and TAV-mCD80-137L viruses, and infected cells were stained for CD80 and CD137L with immunocytochemistry. As with the ADS-12 cells, both CD80 and CD137L were expressed after infection with TAV-mCD80-137L (FIG. 3 and FIG. 4).

A549 cells (human lung carcinoma cells), WI-38 cells (non-cancerous human lung fibroblasts), and MRC5 cells (non-cancerous human lung fibroblasts) were infected with the TAV-A19k and TAV-hCD80-137L viruses, and infected cells were stained for CD80 and CD137L with immunocytochemistry. As depicted in FIG. 5, the TAV-hCD80-137L virus induced expression of human CD80 and human CD137L in cancerous A549 cells with little to no expression in non-cancerous WI-38 and MRC5 cells. These results demonstrate that dual transgene expression can be achieved in human as well as murine cells, and that transgene expression can be selective for cancerous cells.

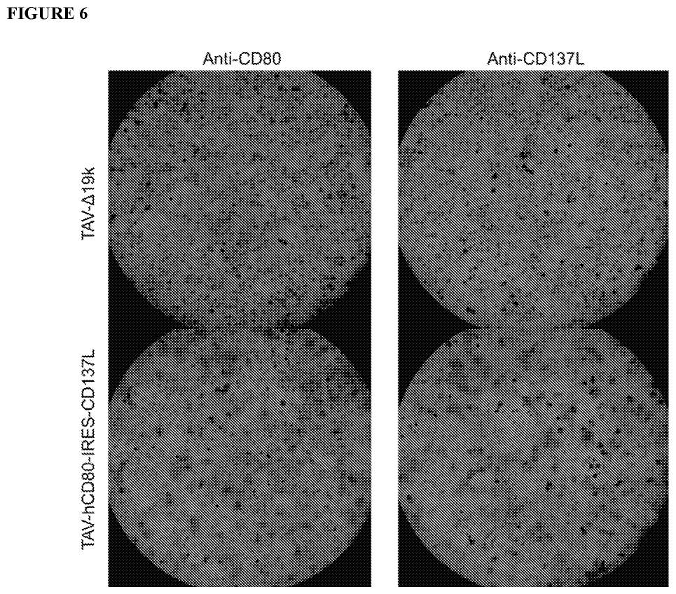

A549 cells (human lung carcinoma cells) were infected with the TAV-Δ19k and TAV-hCD80-IRES-137L viruses, and infected cells were stained for CD80 and CD137L with immunocytochemistry. As depicted in FIG. 6, the TAV-hCD80-IRES-137L virus induced expression of both human CD80 and human CD137L in cancerous A549 cells. These results demonstrate dual transgene expression can be achieved by inserting both transgenes into a single genome region, e.g., the E1b-19k region, separated by an internal ribosome entry site (IRES).

### Example 3: Cytotoxicity of CD80 and CD137L Expressing Adenoviruses

This Example describes the cytotoxicity of CD80 and CD137L expressing recombinant adenoviruses produced as described in Example 1

A549 cells (human lung carcinoma cells), WI-38 cells (non-cancerous human lung fibroblasts), and MRC5 cells (non-cancerous human lung fibroblasts) were infected with the TAV-Δ19k and TAV-hCD80-137L viruses, and infected cells were stained with crystal violet, which stains viable cells blue, at the indicated time points after infection.

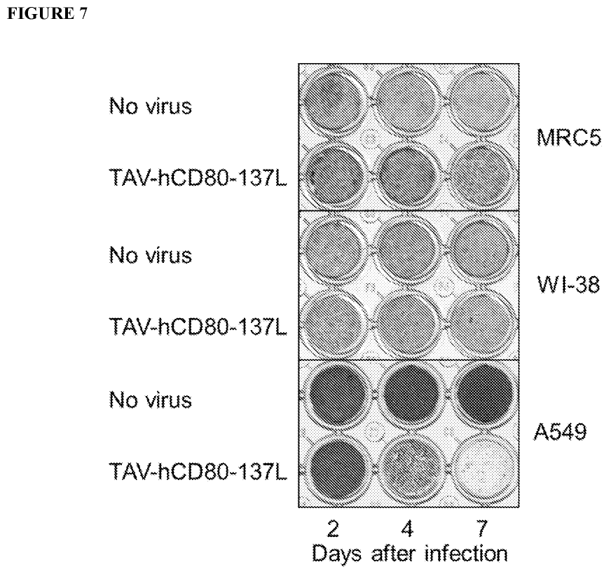

As depicted in FIG. 7, TAV-hCD80-137L was lytic in A549 but not WI-38 or MRC5 cells. These results demonstrate that the TAV-hCD80-137L virus can selectively lyse cancerous cells compared to non-cancerous cells.

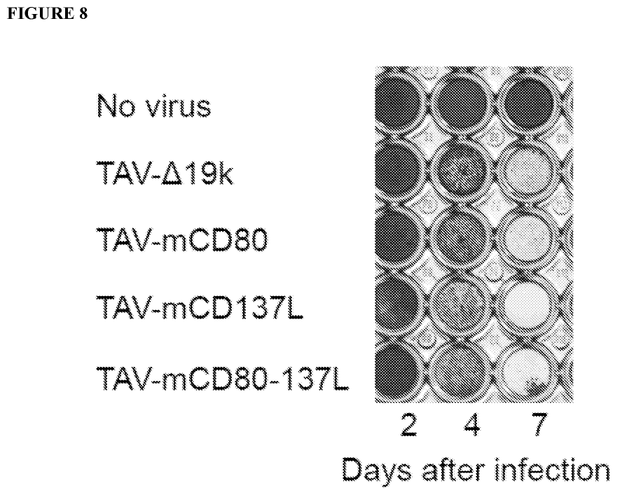

ADS-12 cells were infected with the TAV-A19k, TAV-mCD80, TAV-mCD137L, and TAV-mCD80-137L viruses, and infected cells were stained with crystal violet, which stains viable cells blue, at the indicated time points after infection. Results, depicted in FIG. 8, demonstrate that the TAV-mCD80, TAV-mCD137L, and TAV-mCD80-137L viruses can selectively lyse cancerous cells compared to non-cancerous cells.

### Example 4: Replication of CD80 and CD137L Expressing Adenoviruses

This Example describes the replication in cells of CD80 and CD137L expressing recombinant adenoviruses produced as described in Example 1 in cancerous cells.

ADS cells were infected in triplicate with TAV-A19k, TAV-CD80, TAV-CD137L and TAV-CD80-137L viruses at a MOI of 1. Cells and media were harvested five days after infection and virus titer was determined by plaque assay.

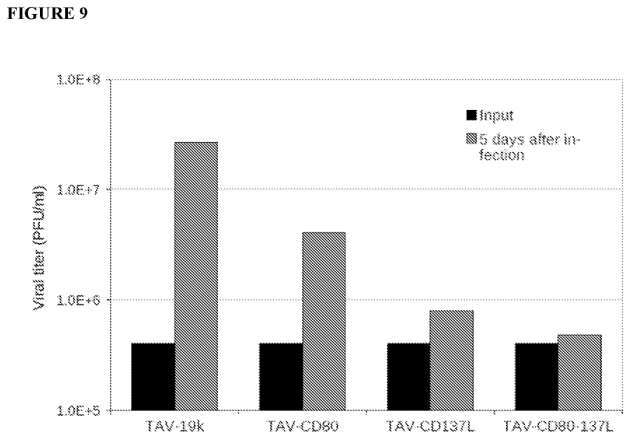

As depicted in FIG. 9, the viruses can effectively replicate in cancerous cells.

### Example 5: Anti-Cancer Activity of CD80 and CD137L Expressing Adenoviruses

This example describes the anti-cancer activity of CD80 and/or CD137L expressing recombinant adenoviruses produced as described in Example 1.

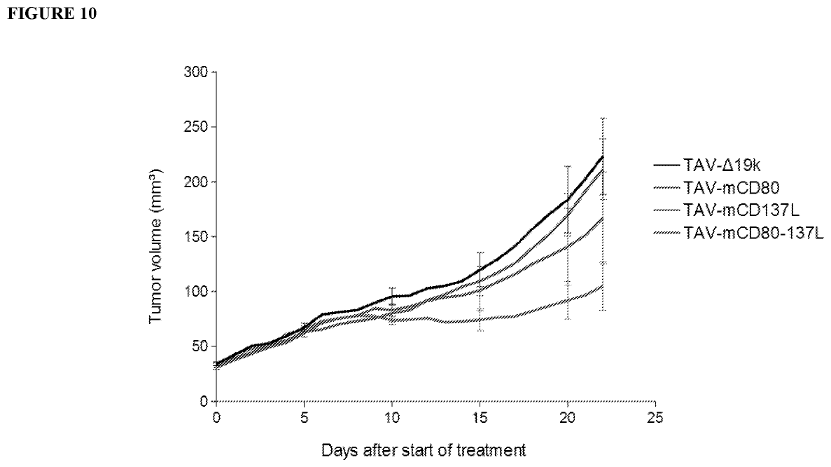

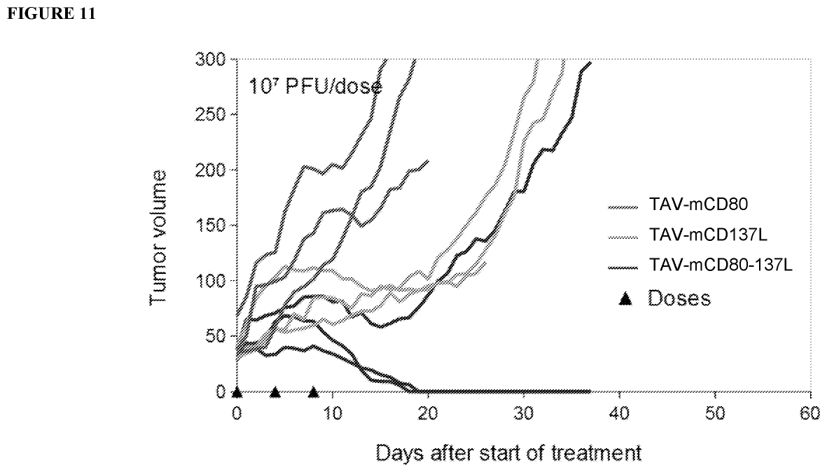

129S4 mice carrying ADS-12 tumors were treated with three intratumoral injections of TAV-Δ19k, TAV-mCD80, TAV-mCD137L, or TAV-mCD80-137L. Results are depicted in FIG. 10. Mice treated with TAV-mCD80 had comparable tumor growth to mice treated with TAV-Δ19k. Mice treated with TAV-mCD137L showed a trend toward smaller tumor size that did not reach statistical significance, and tumors of mice treated with TAV-mCD80-137L were significantly smaller. These results demonstrate that the dual-gene adenovirus expressing CD80 and 137L was most effective in reducing tumor size.

In a separate experiment, 129S4 mice carrying ADS-12 tumors were treated with three intratumoral injections of TAV-Δ19k, TAV-mCD80, TAV-mCD137L, or TAV-mCD80-137L. Results are depicted in FIG. 11. Mice treated with TAV-mCD80-137L had smaller tumor size. These results demonstrate that the dual-gene adenovirus expressing CD80 and 137L was most effective in reducing tumor size.

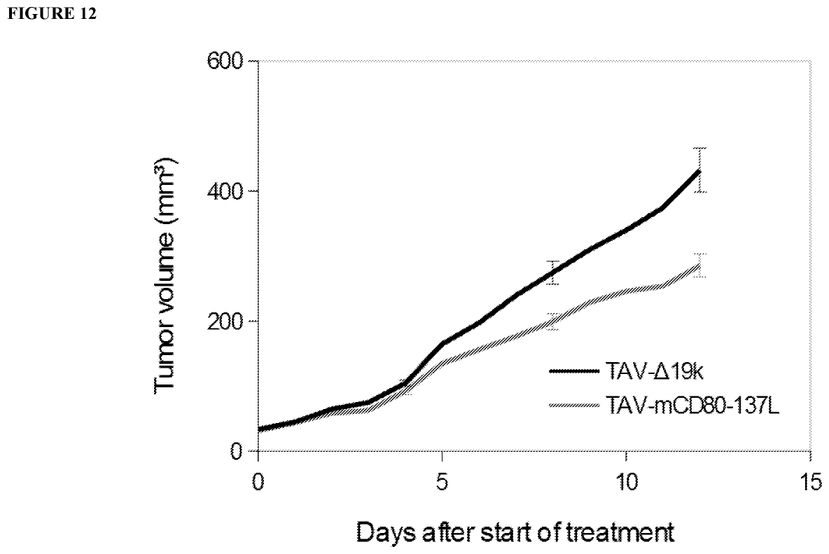

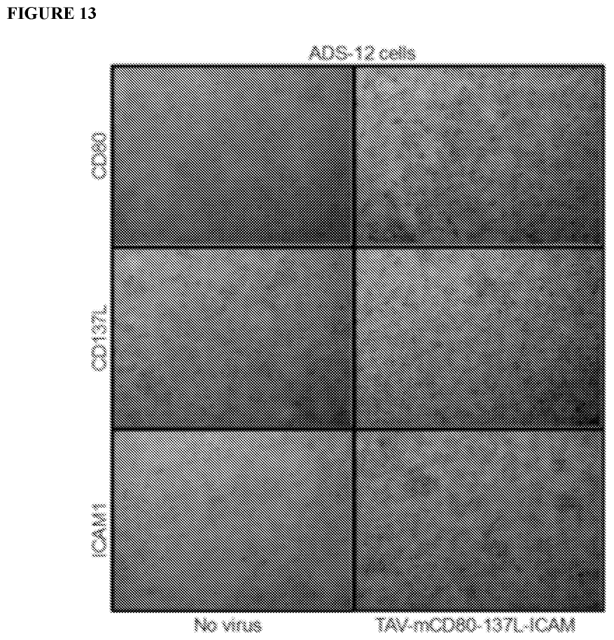

BALB/c mice carrying 4T1 tumors orthotopically implanted in the mammary fat pad were treated with three intratumoral doses of TAV-Δ19k or TAV-mCD80-137L. Again, mice treated with TAV-mCD80-137L had significantly smaller tumors than mice treated with the control virus TAV-Δ19k (FIG. 12).

### Example 6: Construction of a CD80, CD137L, and ICAM-1 Expressing Adenovirus

This Example describes the production of a recombinant adenovirus type 5 (Ad5) that expresses the murine forms of CD80, CD137L, and ICAM-1. ICAM-1 is an intracellular adhesion molecule that is expressed by antigen presenting cells (APCs) and stabilizes interactions between APCs and T-cells by binding to LFA1 on the T cell surface

An adenovirus type 5 virus was constructed that carried the deletion of a nucleotide region located from −304 to −255 upstream of the E1a initiation, which renders E1a expression cancer-selective (as previously described in U.S. Pat. No. 9,073,980). The resulting virus is hereafter referred to as TAV.

TAV was further modified to carry a SalI site at the start site of the E1b-19k region and an XhoI site 200 base pairs 3′ of the SalI site to facilitate insertion of therapeutic transgenes. The nucleotide sequence of the modified E1b-19k region is as follows, with the residual bases from the fused SalI and XhoI sites underlined:

(SEQ ID NO: 15)
ATCTTGGTTACATCTGACCTCGTCGAGTCACCAGGCGCTTTTCCAA

TAV was further modified to delete the adenoviral death protein (ADP), RIDα, RIDβ, and 14.7k genes from the E3 region. The nucleotide sequence of the modified E3 region is as follows, with the hyphen indicating the point of deletion:

(SEQ ID NO: 24)
TTATTGAGGAAAAGAAAATGCCTTAA-
TAAAAAAAAATAATAAAGCATCACTTAC.

TAV was further modified to delete the E4 region except for E4-ORF6/7. The nucleotide sequence of the modified E4 region is as follows, with the hyphen indicating the point of deletion:

(SEQ ID NO: 25)
GAACGCCGGACGTAGTCAT-AACAGTCAGCCTTACCAGTAAA.

The protein coding region of murine CD80 (mCD80), followed by the EMCV IRES, followed by the protein coding region of murine CD137L (mCD137L), followed by the FMDV IRES, followed by the protein coding region of murine ICAM-1 (mICAM-1) was cloned in to the E1b-19k site. The resulting virus is hereafter referred to as TAV-mCD80-137L-ICAM.

The nucleotide sequence of the mCD80-EMCV IRES-137L-FMDV IRES-ICAM insert in the E1b-19k region is as follows, where the coding regions are capitalized, the IRESs are lowercase, and the flanking E1b-19k sequence including the SalI and XhoI restriction sites is underlined:

(SEQ ID NO: 26)
ATCTGACCTCGTCGACATGGCTTGCAATTGTCAGTTGATGCAGGATACAC
CACTCCTCAAGTTTCCATGTCCAAGGCTCATTCTTCTCTTTGTGCTGCTG
ATTCGTCTTTCACAAGTGTCTTCAGATGTTGATGAACAACTGTCCAAGTC
AGTGAAAGATAAGGTATTGCTGCCTTGCCGTTACAACTCTCCTCATGAAG
ATGAGTCTGAAGACCGAATCTACTGGCAAAAACATGACAAAGTGGTGCTG
TCTGTCATTGCTGGGAAACTAAAAGTGTGGCCCGAGTATAAGAACCGGAC
TTTATATGACAACACTACCTACTCTCTTATCATCCTGGGCCTGGTCCTTT
CAGACCGGGGCACATACAGCTGTGTCGTTCAAAAGAAGGAAAGAGGAACG
TATGAAGTTAAACACTTGGCTTTAGTAAAGTTGTCCATCAAAGCTGACTT
CTCTACCCCCAACATAACTGAGTCTGGAAACCCATCTGCAGACACTAAAA
GGATTACCTGCTTTGCTTCCGGGGGTTTCCCAAAGCCTCGCTTCTCTTGG
TTGGAAAATGGAAGAGAATTACCTGGCATCAATACGACAATTTCCCAGGA
TCCTGAATCTGAATTGTACACCATTAGTAGCCAACTAGATTTCAATACGA
CTCGCAACCACACCATTAAGTGTCTCATTAAATATGGAGATGCTCACGTG
TCAGAGGACTTCACCTGGGAAAAACCCCCAGAAGACCCTCCTGATAGCAA
GAACACACTTGTGCTCTTTGGGGCAGGATTCGGCGCAGTAATAACAGTCG
TCGTCATCGTTGTCATCATCAAATGCTTCTGTAAGCACAGAAGCTGTTTC
AGAAGAAATGAGGCAAGCAGAGAAACAAACAACAGCCTTACCTTCGGGCC
TGAAGAAGCATTAGCTGAACAGACCGTCTTCCTTTAGtaacgttactggc
cgaagccgcttggaataaggccggtgtgcgtttgtctatatgttattttc
caccatattgccgtcttttggcaatgtgagggcccggaaacctggccctg
tcttcttgacgagcattcctaggggtctttcccctctcgccaaaggaatg
caaggtctgttgaatgtcgtgaaggaagcagttcctctggaagcttcttg
aagacaaacaacgtctgtagcgaccctttgcaggcagcggaaccccccac
ctggcgacaggtgcctctgcggccaaaagccacgtgtataagatacacct
gcaaaggcggcacaaccccagtgccacgttgtgagttggatagttgtgga
aagagtcaaatggctctcctcaagcgtattcaacaaggggctgaaggatg
cccagaaggtaccccattgtatgggatctgatctggggcctcggtgcaca
tgctttacatgtgtttagtcgaggttaaaaaacgtctaggccccccgaac
cacggggacgtggttttcctttgaaaaacacgatgataatATGGACCAGC
ACACACTTGATGTGGAGGATACCGCGGATGCCAGACATCCAGCAGGTACT
TCGTGCCCCTCGGATGCGGCGCTCCTCAGAGATACCGGGCTCCTCGCGGA
CGCTGCGCTCCTCTCAGATACTGTGCGCCCCACAAATGCCGCGCTCCCCA
CGGATGCTGCCTACCCTGCGGTTAATGTTCGGGATCGCGAGGCCGCGTGG
CCGCCTGCACTGAACTTCTGTTCCCGCCACCCAAAGCTCTATGGCCTAGT
CGCTTTGGTTTTGCTGCTTCTGATCGCCGCCTGTGTTCCTATCTTCACCC
GCACCGAGCCTCGGCCAGCGCTCACAATCACCACCTCGCCCAACCTGGGT
ACCCGAGAGAATAATGCAGACCAGGTCACCCCTGTTTCCCACATTGGCTG
CCCCAACACTACACAACAGGGCTCTCCTGTGTTCGCCAAGCTACTGGCTA
AAAACCAAGCATCGTTGTGCAATACAACTCTGAACTGGCACAGCCAAGAT
GGAGCTGGGAGCTCATACCTATCTCAAGGTCTGAGGTACGAAGAAGACAA
AAAGGAGTTGGTGGTAGACAGTCCCGGGCTCTACTACGTATTTTTGGAAC
TGAAGCTCAGTCCAACATTCACAAACACAGGCCACAAGGTGCAGGGCTGG
GTCTCTCTTGTTTTGCAAGCAAAGCCTCAGGTAGATGACTTTGACAACTT
GGCCCTGACAGTGGAACTGTTCCCTTGCTCCATGGAGAACAAGTTAGTGG
ACCGTTCCTGGAGTCAACTGTTGCTCCTGAAGGCTGGCCACCGCCTCAGT
GTGGGTCTGAGGGCTTATCTGCATGGAGCCCAGGATGCATACAGAGACTG
GGAGCTGTCTTATCCCAACACCACCAGCTTTGGACTCTTTCTTGTGAAAC
CCGACAACCCATGGGAATGAggtttccacaactgataaaactcgtgcaac
ttgaaactccgcctggtctttccaggtctagaggggttacactttgtact
gtgctcgactccacgcccggtccactggcgggtgttagtagcagcactgt
tgtttcgtagcggagcatggtggccgtgggaactcctccttggtgacaag
ggcccacggggccgaaagccacgtccagacggacccaccatgtgtgcaac
cccagcacggcaacttttactgcgaacaccaccttaaggtgacactggta
ctggtactcggtcactggtgacaggctaaggatgcccttcaggtaccccg
aggtaacacgggacactcgggatctgagaaggggattgggacttctttaa
aagtgcccagtttaaaaagcttctacgcctgaataggcgaccggaggccg
gcgcctttccattacccactactaaatccATGGCTTCAACCCGTGCCAAG
CCCACGCTACCTCTGCTCCTGGCCCTGGTCACCGTTGTGATCCCTGGGCC
TGGTGATGCTCAGGTATCCATCCATCCCAGAGAAGCCTTCCTGCCCCAGG
GTGGGTCCGTGCAGGTGAACTGTTCTTCCTCATGCAAGGAGGACCTCAGC
CTGGGCTTGGAGACTCAGTGGCTGAAAGATGAGCTCGAGAGTGGACCCAA
CTGGAAGCTGTTTGAGCTGAGCGAGATCGGGGAGGACAGCAGTCCGCTGT
GCTTTGAGAACTGTGGCACCGTGCAGTCGTCCGCTTCCGCTACCATCACC
GTGTATTCGTTTCCGGAGAGTGTGGAGCTGAGACCTCTGCCAGCCTGGCA
GCAAGTAGGCAAGGACCTCACCCTGCGCTGCCACGTGGATGGTGGAGCAC
CGCGGACCCAGCTCTCAGCAGTGCTGCTCCGTGGGGAGGAGATACTGAGC
CGCCAGCCAGTGGGTGGGCACCCCAAGGACCCCAAGGAGATCACATTCAC
GGTGCTGGCTAGCAGAGGGGACCACGGAGCCAATTTCTCATGCCGCACAG
AACTGGATCTCAGGCCGCAAGGGCTGGCATTGTTCTCTAATGTCTCCGAG
GCCAGGAGCCTCCGGACTTTCGATCTTCCAGCTACCATCCCAAAGCTCGA
CACCCCTGACCTCCTGGAGGTGGGCACCCAGCAGAAGTTGTTTTGCTCCC
TGGAAGGCCTGTTTCCTGCCTCTGAAGCTCGGATATACCTGGAGCTGGGA
GGCCAGATGCCGACCCAGGAGAGCACAAACAGCAGTGACTCTGTGTCAGC
CACTGCCTTGGTAGAGGTGACTGAGGAGTTCGACAGAACCCTGCCGCTGC
GCTGCGTTTTGGAGCTAGCGGACCAGATCCTGGAGACGCAGAGGACCTTA
ACAGTCTACAACTTTTCAGCTCCGGTCCTGACCCTGAGCCAGCTGGAGGT
CTCGGAAGGGAGCCAAGTAACTGTGAAGTGTGAAGCCCACAGTGGGTCGA
AGGTGGTTCTTCTGAGCGGCGTCGAGCCTAGGCCACCCACCCCGCAGGTC
CAATTCACACTGAATGCCAGCTCGGAGGATCACAAACGAAGCTTCTTTTG
CTCTGCCGCTCTGGAGGTGGCGGGAAAGTTCCTGTTTAAAAACCAGACCC
TGGAACTGCACGTGCTGTATGGTCCTCGGCTGGACGAGACGGACTGCTTG
GGGAACTGGACCTGGCAAGAGGGGTCTCAGCAGACTCTGAAATGCCAGGC
CTGGGGGAACCCATCTCCTAAGATGACCTGCAGACGGAAGGCAGATGGTG
CCCTGCTGCCCATCGGGGTGGTGAAGTCTGTCAAACAGGAGATGAATGGT
ACATACGTGTGCCATGCCTTTAGCTCCCATGGGAATGTCACCAGGAATGT
GTACCTGACAGTACTGTACCACTCTCAAAATAACTGGACTATAATCATTC
TGGTGCCAGTACTGCTGGTCATTGTGGGCCTCGTGATGGCAGCCTCTTAT
GTTTATAACCGCCAGAGAAAGATCAGGATATACAAGTTACAGAAGGCTCA
GGAGGAGGCCATAAAACTCAAGGGACAAGCCCCACCTCCCTGACTCGAGT
CACCAGGCG.

Additionally, the protein coding region of human CD80 (hCD80), followed by the EMCV IRES, followed by the protein coding region of human CD137L (hCD137L), followed by the FMDV IRES, followed by the protein coding region of human ICAM-1 (hICAM-1) is cloned in to the E1b-19k site. The resulting virus is hereafter referred to as TAV-hCD80-137L-ICAM.

The nucleotide sequence of the hCD80-EMCV IRES-137L-FMDV IRES-ICAM insert in the E1b-19k region is as follows, where the coding regions are capitalized, the IRESs are lowercase, and the flanking E1b-19k sequence including the SalI and XhoI restriction sites is underlined:

(SEQ ID NO: 31)
ATCTGACCTCGTCGACATGGGCCACACACGGAGGCAGGGAACATCACCAT
CCAAGTGTCCATACCTCAATTTCTTTCAGCTCTTGGTGCTGGCTGGTCTT
TCTCACTTCTGTTCAGGTGTTATCCACGTGACCAAGGAAGTGAAAGAAGT
GGCAACGCTGTCCTGTGGTCACAATGTTTCTGTTGAAGAGCTGGCACAAA
CTCGCATCTACTGGCAAAAGGAGAAGAAAATGGTGCTGACTATGATGTCT
GGGGACATGAATATATGGCCCGAGTACAAGAACCGGACCATCTTTGATAT
CACTAATAACCTCTCCATTGTGATCCTGGCTCTGCGCCCATCTGACGAGG
GCACATACGAGTGTGTTGTTCTGAAGTATGAAAAAGACGCTTTCAAGCGG
GAACACCTGGCTGAAGTGACGTTATCAGTCAAAGCTGACTTCCCTACACC
TAGTATATCTGACTTTGAAATTCCAACTTCTAATATTAGAAGGATAATTT
GCTCAACCTCTGGAGGTTTTCCAGAGCCTCACCTCTCCTGGTTGGAAAAT
GGAGAAGAATTAAATGCCATCAACACAACAGTTTCCCAAGATCCTGAAAC
TGAGCTCTATGCTGTTAGCAGCAAACTGGATTTCAATATGACAACCAACC
ACAGCTTCATGTGTCTCATCAAGTATGGACATTTAAGAGTGAATCAGACC
TTCAACTGGAATACAACCAAGCAAGAGCATTTTCCTGATAACCTGCTCCC
ATCCTGGGCCATTACCTTAATCTCAGTAAATGGAATTTTTGTGATATGCT
GCCTGACCTACTGCTTTGCCCCAAGATGCAGAGAGAGAAGGAGGAATGAG
AGATTGAGAAGGGAAAGTGTACGCCCTGTATAAtaacgttactggccgaa
gccgcttggaataaggccggtgtgcgtttgtctatatgttattttccacc
atattgccgtcttttggcaatgtgagggcccggaaacctggccctgtctt
cttgacgagcattcctaggggtctttcccctctcgccaaaggaatgcaag
gtctgttgaatgtcgtgaaggaagcagttcctctggaagcttcttgaaga
caaacaacgtctgtagcgaccctttgcaggcagcggaaccccccacctgg
cgacaggtgcctctgcggccaaaagccacgtgtataagatacacctgcaa
aggcggcacaaccccagtgccacgttgtgagttggatagttgtggaaaga
gtcaaatggctctcctcaagcgtattcaacaaggggctgaaggatgccca
gaaggtaccccattgtatgggatctgatctggggcctcggtgcacatgct
ttacatgtgtttagtcgaggttaaaaaacgtctaggccccccgaaccacg
gggacgtggttttcctttgaaaaacacgatgataatATGGAATACGCCTC
TGACGCTTCACTGGACCCCGAAGCCCCGTGGCCTCCTGCACCTCGCGCTC
GCGCCTGCCGCGTACTGCCTTGGGCCCTGGTCGCGGGGCTGCTGCTCCTG
CTCCTGCTCGCTGCTGCATGCGCTGTATTTCTTGCATGCCCATGGGCTGT
GTCTGGGGCTCGCGCATCACCTGGCTCCGCGGCCAGCCCGAGACTCCGCG
AGGGTCCCGAGCTTTCGCCCGACGATCCCGCCGGCCTCTTGGACCTGCGG
CAGGGCATGTTTGCGCAGCTGGTGGCCCAAAATGTTCTGCTGATCGATGG
GCCCCTGAGCTGGTACAGTGACCCAGGCCTGGCAGGCGTGTCCCTGACGG
GGGGCCTGAGCTACAAAGAGGACACGAAGGAGCTGGTGGTGGCCAAGGCT
GGAGTCTACTATGTCTTCTTTCAACTAGAGCTGCGGCGCGTGGTGGCCGG
CGAGGGCTCAGGCTCCGTTTCACTTGCGCTGCACCTGCAGCCACTGCGCT
CTGCTGCTGGGGCCGCCGCCCTGGCTTTGACCGTGGACCTGCCACCCGCC
TCCTCCGAGGCTCGGAACTCGGCCTTCGGTTTCCAGGGCCGCTTGCTGCA
CCTGAGTGCCGGCCAGCGCCTGGGCGTCCATCTTCACACTGAGGCCAGGG
CACGCCATGCCTGGCAGCTTACCCAGGGCGCCACAGTCTTGGGACTCTTC
CGGGTGACCCCCGAAATCCCAGCCGGACTCCCTTCACCGAGGTCGGAATA
Aggtttccacaactgataaaactcgtgcaacttgaaactccgcctggtct
ttccaggtctagaggggttacactttgtactgtgctcgactccacgcccg
gtccactggcgggtgttagtagcagcactgttgtttcgtagcggagcatg
gtggccgtgggaactcctccttggtgacaagggcccacggggccgaaagc
cacgtccagacggacccaccatgtgtgcaaccccagcacggcaactttta
ctgcgaacaccaccttaaggtgacactggtactggtactcggtcactggt
gacaggctaaggatgcccttcaggtaccccgaggtaacacgggacactcg
ggatctgagaaggggattgggacttctttaaaagtgcccagtttaaaaag
cttctacgcctgaataggcgaccggaggccggcgcctttccattacccac
tactaaatccATGGCTCCCAGCAGCCCCCGGCCCGCGCTGCCCGCACTCC
TGGTCCTGCTCGGGGCTCTGTTCCCAGGACCTGGCAATGCCCAGACATCT
GTGTCCCCCTCAAAAGTCATCCTGCCCCGGGGAGGCTCCGTGCTGGTGAC
ATGCAGCACCTCCTGTGACCAGCCCAAGTTGTTGGGCATAGAGACCCCGT
TGCCTAAAAAGGAGTTGCTCCTGCCTGGGAACAACCGGAAGGTGTATGAA
CTGAGCAATGTGCAAGAAGATAGCCAACCAATGTGCTATTCAAACTGCCC
TGATGGGCAGTCAACAGCTAAAACCTTCCTCACCGTGTACTGGACTCCAG
AACGGGTGGAACTGGCACCCCTCCCCTCTTGGCAGCCAGTGGGCAAGAAC
CTTACCCTACGCTGCCAGGTGGAGGGTGGGGCACCCCGGGCCAACCTCAC
CGTGGTGCTGCTCCGTGGGGAGAAGGAGCTGAAACGGGAGCCAGCTGTGG
GGGAGCCCGCTGAGGTCACGACCACGGTGCTGGTGAGGAGAGATCACCAT
GGAGCCAATTTCTCGTGCCGCACTGAACTGGACCTGCGGCCCCAAGGGCT
GGAGCTGTTTGAGAACACCTCGGCCCCCTACCAGCTCCAGACCTTTGTCC
TGCCAGCGACTCCCCCACAACTTGTCAGCCCCCGGGTCCTAGAGGTGGAC
ACGCAGGGGACCGTGGTCTGTTCCCTGGACGGGCTGTTCCCAGTCTCGGA
GGCCCAGGTCCACCTGGCACTGGGGGACCAGAGGTTGAACCCCACAGTCA
CCTATGGCAACGACTCCTTCTCGGCCAAGGCCTCAGTCAGTGTGACCGCA
GAGGACGAGGGCACCCAGCGGCTGACGTGTGCAGTAATACTGGGGAACCA
GAGCCAGGAGACACTGCAGACAGTGACCATCTACAGCTTTCCGGCGCCCA
ACGTGATTCTGACGAAGCCAGAGGTCTCAGAAGGGACCGAGGTGACAGTG
AAGTGTGAGGCCCACCCTAGAGCCAAGGTGACGCTGAATGGGGTTCCAGC
CCAGCCACTGGGCCCGAGGGCCCAGCTCCTGCTGAAGGCCACCCCAGAGG
ACAACGGGCGCAGCTTCTCCTGCTCTGCAACCCTGGAGGTGGCCGGCCAG
CTTATACACAAGAACCAGACCCGGGAGCTTCGTGTCCTGTATGGCCCCCG
ACTGGACGAGAGGGATTGTCCGGGAAACTGGACGTGGCCAGAAAATTCCC
AGCAGACTCCAATGTGCCAGGCTTGGGGGAACCCATTGCCCGAGCTCAAG
TGTCTAAAGGATGGCACTTTCCCACTGCCCATCGGGGAATCAGTGACTGT
CACTCGAGATCTTGAGGGCACCTACCTCTGTCGGGCCAGGAGCACTCAAG
GGGAGGTCACCCGCAAGGTGACCGTGAATGTGCTCTCCCCCCGGTATGAG
ATTGTCATCATCACTGTGGTAGCAGCCGCAGTCATAATGGGCACTGCAGG
CCTCAGCACGTACCTCTATAACCGCCAGCGGAAGATCAAGAAATACAGAC
TACAACAGGCCCAAAAAGGGACCCCCATGAAACCGAACACACAAGCCACG
CCTCCCTGACTCGAGTCACCAGGCG.

### Example 7: CD80, CD137L, and ICAM-1 Gene Expression

This example describes the expression of CD80, CD137L, and ICAM-1 from the recombinant adenovirus produced as described in Example 6.

ADS-12 cells (mouse lung adenocarcinoma cells) were infected with the TAV-mCD80-137L-ICAM virus at a MOI of 10 or kept as non-infected controls and stained four days after infection for CD80, CD137L, and ICAM-1 by immunocytochemistry. As depicted in FIG. 13, each gene was expressed with the TAV-mCD80-137L-ICAM virus, demonstrating that the single virus drove expression of three therapeutic genes.

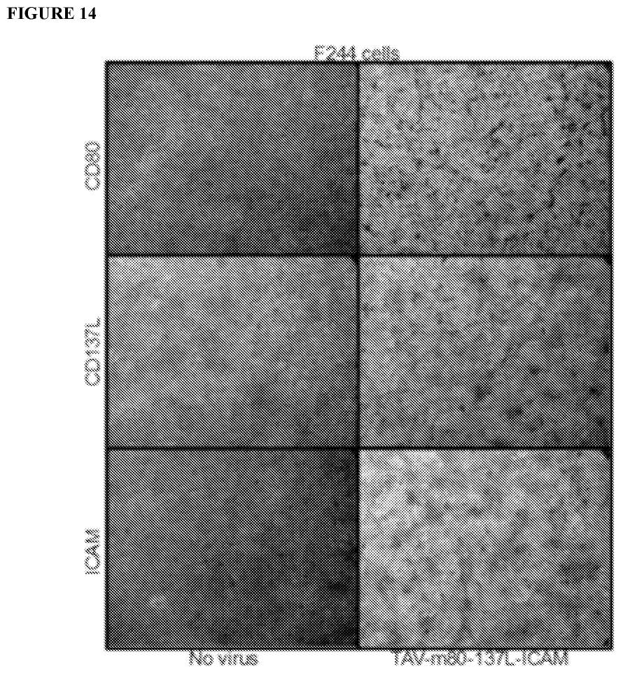

F244 cells (mouse sarcoma cells) were infected with the TAV-mCD80-137L-ICAM virus at a MOI of 5 or kept as non-infected controls and stained three days after infection for CD80, CD137L, and ICAM-1 by immunocytochemistry. As depicted in FIG. 14, each gene was expressed with the TAV-mCD80-137L-ICAM virus, demonstrating that the single virus drove expression of three therapeutic genes.

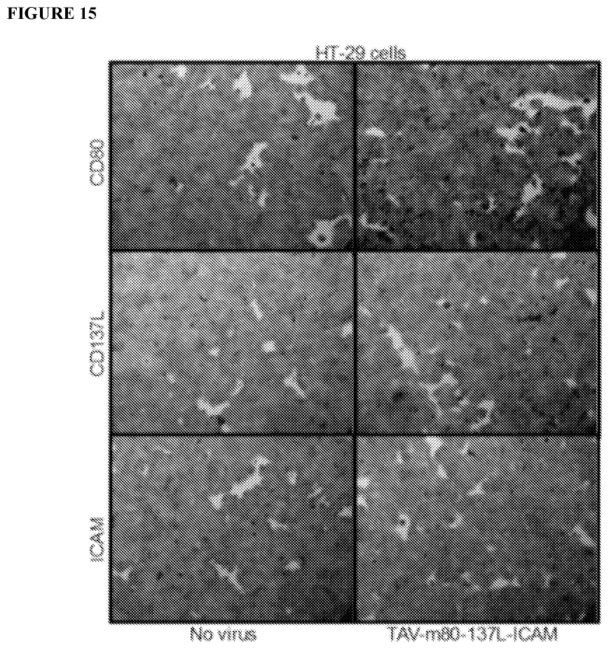

HT29 (human colorectal adenocarcinoma cells) were infected with the TAV-mCD80-mCD137L-mICAM-1 virus at a MOI of 5 or kept as non-infected controls and stained three days after infection for CD80, CD137L, and ICAM-1 by immunocytochemistry. As depicted in FIG. 15, each gene was expressed with the TAV-mCD80-137L-ICAM virus, demonstrating that the single virus drove expression of three therapeutic genes.

### Example 8: Anti-Cancer Activity of CD80, CD137L, and ICAM-1 Expressing Adenoviruses

This example describes the anti-cancer activity of CD80 and CD137L expressing recombinant adenoviruses and CD80, CD137L, and ICAM-1 expressing adenoviruses.

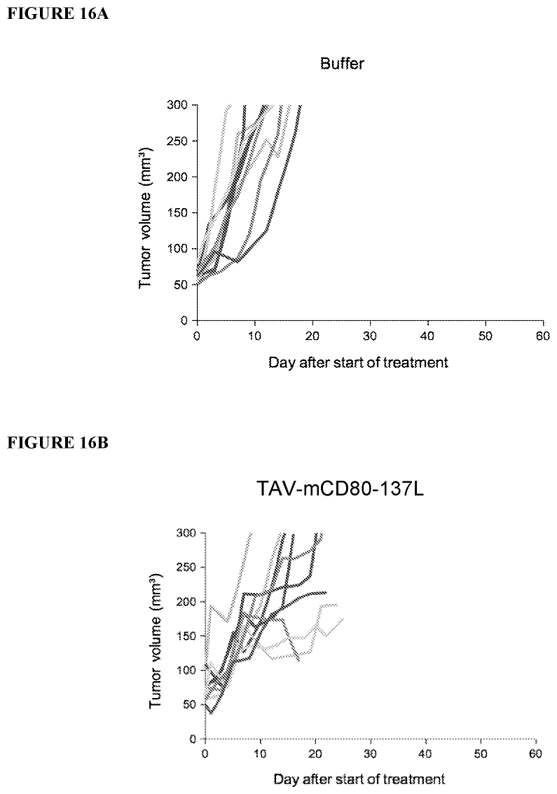

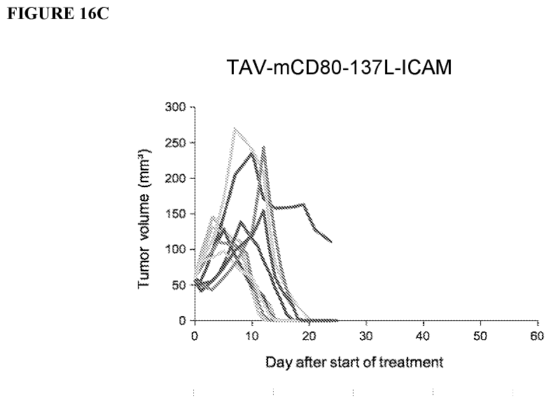

129S4 mice carrying ADS-12 tumors were treated with three intratumoral injections of buffer, TAV-mCD80-137L (produced as described in Example 1), or TAV-mCD80-137L-ICAM (produced as described in Example 6). Results are depicted in FIG. 16. Tumors in mice treated with TAV-mCD80-137L were smaller than those treated with buffer. Tumors of mice treated with TAV-mCD80-137L-ICAM were smaller than those treated with TAV-mCD80-m137L or buffer, with many mice showing complete loss of tumor volume. These results demonstrate that CD80 and 137L expressing viruses and CD80, CD137L, and mICAM-1 expressing viruses are effective in reducing tumor size.

### Example 9: Construction of Endostatin and Angiostatin Expressing Adenoviruses

This Example describes the construction of a recombinant adenovirus type 5 (Ad5) that expresses endostatin and angiostatin.

A plasmid carrying the 5′ portion of the adenovirus type 5 genomic sequence is modified to carry the deletion of a nucleotide region located from −304 to −255 upstream of the E1a initiation site, which renders E1a expression cancer-selective (as previously described in U.S. Pat. No. 9,073,980). The modified plasmid is hereafter referred to as the TAV plasmid, and any resulting viral particles produced therefrom are hereafter referred to as the TAV virus.

The TAV plasmid is further modified to carry a SalI site at the start of the E1b-19k region and an XhoI site 200 base pairs 3′ of the SalI site to facilitate insertion of therapeutic transgenes. To delete the 200 base pair E1b-19k region the plasmid is cut with SalI and XhoI and self-ligated. The nucleotide sequence of the modified E1b-19k region is as follows, with the residual bases from the fused SalI and XhoI sites underlined:

(SEQ ID NO: 15)
ATCTTGGTTACATCTGACCTCGTCGAGTCACCAGGCGCTTTTCCAA.

Additionally, a nucleotide sequence encoding amino acid residues 1-23 of human collagen XVIII (corresponding to the signal peptide) followed by residues 1318-1516 of human collagen XVIII (corresponding to a C-terminal fragment) followed by an encephalomyocarditis virus (EMCV) IRES followed by a nucleotide sequence encoding amino acid residues 1-19 of human plasminogen (corresponding to the signal peptide) followed by residues 97-549 of human plasminogen (corresponding to kringle domains 1-5) is cloned in to the modified E1b-19k region. All human collagen XVIII amino acid residue numbers are relative to NCBI Reference Sequence: NP_085059.2, depicted herein as SEQ ID NO: 33. All human plasminogen amino acid residue numbers are relative to NCBI Reference Sequence: NP_000292.1, depicted herein as SEQ ID NO: 34. The modified plasmid is hereafter referred to as the TAV-hEndo-IRES-hAng plasmid, and any resulting viral particles produced therefrom are hereafter referred to as the TAV-hEndo-IRES-hAng virus. The nucleotide sequence of the TAV-hEndo-IRES-hAng plasmid in the E1b-19k region is as follows, where the coding regions are capitalized, the IRES is lowercase, and the flanking E1b-19k sequence including the SalI and XhoI restriction sites is underlined:

(SEQ ID NO: 35)
ATCTGACCTCGTCGACATGGCTCCCTACCCCTGTGGCTGCCACATCCTG
CTGCTGCTCTTCTGCTGCCTGGCGGCTGCCCGGGCCAGCTCCTACGTGC
ACCTGCGGCCGGCGCGACCCACAAGCCCACCCGCCCACAGCCACCGCGA
CTTCCAGCCGGTGCTCCACCTGGTTGCGCTCAACAGCCCCCTGTCAGGC
GGCATGCGGGGCATCCGCGGGGCCGACTTCCAGTGCTTCCAGCAGGCGC
GGGCCGTGGGGCTGGCGGGCACCTTCCGCGCCTTCCTGTCCTCGCGCCT
GCAGGACCTGTACAGCATCGTGCGCCGTGCCGACCGCGCAGCCGTGCCC
ATCGTCAACCTCAAGGACGAGCTGCTGTTTCCCAGCTGGGAGGCTCTGT
TCTCAGGCTCTGAGGGTCCGCTGAAGCCCGGGGCACGCATCTTCTCCTT
TGACGGCAAGGACGTCCTGAGGCACCCCACCTGGCCCCAGAAGAGCGTG
TGGCATGGCTCGGACCCCAACGGGCGCAGGCTGACCGAGAGCTACTGTG
AGACGTGGCGGACGGAGGCTCCCTCGGCCACGGGCCAGGCCTCCTCGCT
GCTGGGGGGCAGGCTCCTGGGGCAGAGTGCCGCGAGCTGCCATCACGCC
TACATCGTGCTCTGCATTGAGAACAGCTTCATGACTGCCTCCAAGTAGt
aacgttactggccgaagccgcttggaataaggccggtgtgcgtttgtct
atatgttattttccaccatattgccgtcttttggcaatgtgagggcccg
gaaacctggccctgtcttcttgacgagcattcctaggggtctttcccct
ctcgccaaaggaatgcaaggtctgttgaatgtcgtgaaggaagcagttc
ctctggaagcttcttgaagacaaacaacgtctgtagcgaccctttgcag
gcagcggaaccccccacctggcgacaggtgcctctgcggccaaaagcca
cgtgtataagatacacctgcaaaggcggcacaaccccagtgccacgttg
tgagttggatagttgtggaaagagtcaaatggctctcctcaagcgtatt
caacaaggggctgaaggatgcccagaaggtaccccattgtatgggatct
gatctggggcctcggtgcacatgctttacatgtgtttagtcgaggttaa
aaaacgtctaggccccccgaaccacggggacgtggttttcctttgaaaa
acacgatgataatATGGAACATAAGGAAGTGGTTCTTCTACTTCTTTTA
TTTCTGAAATCAGGTCAAGGAAAAGTGTATCTCTCAGAGTGCAAGACTG
GGAATGGAAAGAACTACAGAGGGACGATGTCCAAAACAAAAAATGGCAT
CACCTGTCAAAAATGGAGTTCCACTTCTCCCCACAGACCTAGATTCTCA
CCTGCTACACACCCCTCAGAGGGACTGGAGGAGAACTACTGCAGGAATC
CAGACAACGATCCGCAGGGGCCCTGGTGCTATACTACTGATCCAGAAAA
GAGATATGACTACTGCGACATTCTTGAGTGTGAAGAGGAATGTATGCAT
TGCAGTGGAGAAAACTATGACGGCAAAATTTCCAAGACCATGTCTGGAC
TGGAATGCCAGGCCTGGGACTCTCAGAGCCCACACGCTCATGGATACAT
TCCTTCCAAATTTCCAAACAAGAACCTGAAGAAGAATTACTGTCGTAAC
CCCGATAGGGAGCTGCGGCCTTGGTGTTTCACCACCGACCCCAACAAGC
GCTGGGAACTTTGTGACATCCCCCGCTGCACAACACCTCCACCATCTTC
TGGTCCCACCTACCAGTGTCTGAAGGGAACAGGTGAAAACTATCGCGGG
AATGTGGCTGTTACCGTGTCCGGGCACACCTGTCAGCACTGGAGTGCAC
AGACCCCTCACACACATAACAGGACACCAGAAAACTTCCCCTGCAAAAA
TTTGGATGAAAACTACTGCCGCAATCCTGACGGAAAAAGGGCCCCATGG
TGCCATACAACCAACAGCCAAGTGCGGTGGGAGTACTGTAAGATACCGT
CCTGTGACTCCTCCCCAGTATCCACGGAACAATTGGCTCCCACAGCACC
ACCTGAGCTAACCCCTGTGGTCCAGGACTGCTACCATGGTGATGGACAG
AGCTACCGAGGCACATCCTCCACCACCACCACAGGAAAGAAGTGTCAGT
CTTGGTCATCTATGACACCACACCGGCACCAGAAGACCCCAGAAAACTA
CCCAAATGCTGGCCTGACAATGAACTACTGCAGGAATCCAGATGCCGAT
AAAGGCCCCTGGTGTTTTACCACAGACCCCAGCGTCAGGTGGGAGTACT
GCAACCTGAAAAAATGCTCAGGAACAGAAGCGAGTGTTGTAGCACCTCC
GCCTGTTGTCCTGCTTCCAGATGTAGAGACTCCTTCCGAAGAAGACTGT
ATGTTTGGGAATGGGAAAGGATACCGAGGCAAGAGGGCGACCACTGTTA
CTGGGACGCCATGCCAGGACTGGGCTGCCCAGGAGCCCCATAGACACAG
CATTTTCACTCCAGAGACAAATCCACGGGCGGGTCTGGAAAAAAATTAC
TGCCGTAACCCTGATGGTGATGTAGGTGGTCCCTGGTGCTACACGACAA
ATCCAAGATAGCTCGAGTCACCAGGCG.

Additionally, a nucleotide sequence encoding amino acid residues 1-26 of mouse collagen XVIII (corresponding to the signal peptide) followed by residues 1577-1774 of mouse collagen XVIII (corresponding to a C-terminal fragment) followed by an encephalomyocarditis virus (EMCV) IRES followed by a nucleotide sequence encoding amino acid residues 1-19 of mouse plasminogen (corresponding to the signal peptide) followed by residues 96-549 of mouse plasminogen (corresponding to kringle domains 1-5) is cloned in to the modified E1b-19k region. The modified plasmid is hereafter referred to as the TAV-Endo-IRES-Ang plasmid, and any resulting viral particles produced therefrom are hereafter referred to as the TAV-Endo-IRES-Ang virus. The nucleotide sequence of the TAV-Endo-IRES-Ang plasmid in the E1b-19k region is as follows, where the coding regions are capitalized, the IRES is lowercase, and the flanking E1b-19k sequence including the SalI and XhoI restriction sites is underlined:

(SEQ ID NO: 36)
ATCTGACCTCGTCGACATGGCTCCCGACCCCAGCAGACGCCTCTGCCTG
CTGCTGCTGTTGCTGCTCTCCTGCCGCCTTGTGCCTGCCAGCGCTTATG
TGCACCTGCCGCCAGCCCGCCCCACCCTCTCACTTGCTCATACTCATCA
GGACTTTCAGCCAGTGCTCCACCTGGTGGCACTGAACACCCCCCTGTCT
GGAGGCATGCGTGGTATCCGTGGAGCAGATTTCCAGTGCTTCCAGCAAG
CCCGAGCCGTGGGGCTGTCGGGCACCTTCCGGGCTTTCCTGTCCTCTAG
GCTGCAGGATCTCTATAGCATCGTGCGCCGTGCTGACCGGGGGTCTGTG
CCCATCGTCAACCTGAAGGACGAGGTGCTATCTCCCAGCTGGGACTCCC
TGTTTTCTGGCTCCCAGGGTCAACTGCAACCCGGGGCCCGCATCTTTTC
TTTTGACGGCAGAGATGTCCTGAGACACCCAGCCTGGCCGCAGAAGAGC
GTATGGCACGGCTCGGACCCCAGTGGGCGGAGGCTGATGGAGAGTTACT
GTGAGACATGGCGAACTGAAACTACTGGGGCTACAGGTCAGGCCTCCTC
CCTGCTGTCAGGCAGGCTCCTGGAACAGAAAGCTGCGAGCTGCCACAAC
AGCTACATCGTCCTGTGCATTGAGAATAGCTTCATGACCTCTTTCTCCA
AATAGtaacgttactggccgaagccgcttggaataaggccggtgtgcgt
ttgtctatatgttattttccaccatattgccgtcttttggcaatgtgag
ggcccggaaacctggccctgtcttcttgacgagcattcctaggggtctt
tcccctctcgccaaaggaatgcaaggtctgttgaatgtcgtgaaggaag
cagttcctctggaagcttcttgaagacaaacaacgtctgtagcgaccct
ttgcaggcagcggaaccccccacctggcgacaggtgcctctgcggccaa
aagccacgtgtataagatacacctgcaaaggcggcacaaccccagtgcc
acgttgtgagttggatagttgtggaaagagtcaaatggctctcctcaag
cgtattcaacaaggggctgaaggatgcccagaaggtaccccattgtatg
ggatctgatctggggcctcggtgcacatgctttacatgtgtttagtcga
ggttaaaaaacgtctaggccccccgaaccacggggacgtggttttcctt
tgaaaaacacgatgataatATGGACCACAAGGAAGTAATCCTTCTGTTT
CTCTTGCTTCTGAAACCAGGACAAGGGAAGAGAGTGTATCTGTCAGAAT
GTAAGACCGGCATCGGCAACGGCTACAGAGGAACAATGTCCAGGACAAA
GAGTGGTGTTGCCTGTCAAAAGTGGGGTGCCACGTTCCCCCACGTACCC
AACTACTCTCCCAGTACACATCCCAATGAGGGACTAGAAGAAAATTACT
GTAGGAACCCAGACAATGATGAACAAGGGCCTTGGTGCTACACTACAGA
TCCGGACAAGAGATATGACTACTGCAACATTCCTGAATGTGAAGAAGAA
TGCATGTACTGCAGTGGCGAAAAGTATGAGGGGAAAATCTCCAAGACCA
TGTCTGGACTTGACTGCCAGGCCTGGGATTCTCAGAGCCCACATGCTCA
TGGATACATCCCTGCCAAATTCCCAAGCAAGAACCTGAAGATGAATTAT
TGCCGCAACCCTGACGGGGAGCCAAGGCCCTGGTGCTTCACAACAGACC
CCACCAAACGCTGGGAATACTGTGACATCCCCCGCTGCACAACACCCCC
GCCCCCACCCAGCCCAACCTACCAATGTCTGAAAGGAAGAGGTGAAAAT
TACCGAGGGACCGTGTCTGTCACCGTGTCTGGGAAAACCTGTCAGCGCT
GGAGTGAGCAAACCCCTCATAGGCACAACAGGACACCAGAAAATTTCCC
CTGCAAAAATCTGGAGGAGAATTACTGCCGGAACCCGGATGGAGAAACT
GCTCCCTGGTGCTATACCACTGACAGCCAGCTGAGGTGGGAGTACTGTG
AGATTCCATCCTGCGAGTCCTCAGCATCACCAGACCAGTCAGATTCCTC
AGTTCCACCAGAGGAGCAAACACCTGTGGTCCAGGAATGCTACCAGAGC
GATGGGCAGAGCTATCGGGGTACATCGTCCACTACCATCACAGGGAAGA
AGTGCCAGTCCTGGGCAGCTATGTTTCCACATAGGCATTCGAAGACGCC
AGAGAACTTCCCAGATGCTGGCTTGGAGATGAACTATTGCAGGAACCCG
GATGGTGACAAGGGCCCTTGGTGCTACACCACTGACCCGAGCGTCAGGT
GGGAATACTGCAACCTGAAGCGGTGCTCAGAGACAGGAGGGAGTGTTGT
GGAATTGCCCACAGTTTCCCAGGAACCAAGTGGGCCGAGCGACTCTGAG
ACAGACTGCATGTATGGGAATGGCAAAGACTACCGGGGCAAAACGGCCG
TCACTGCAGCTGGCACCCCTTGCCAAGGATGGGCTGCCCAGGAGCCCCA
CAGGCACAGCATCTTCACCCCACAGACAAACCCACGGGCAGGTCTGGAA
AAGAATTATTGCCGAAACCCCGATGGGGATGTGAATGGTCCTTGGTGCT
ATACAACAAACCCTAGATGATAGCTCGAGTCACCAGGCG.

The various plasmids described are used along with other plasmids carrying the remainder of the adenovirus type 5 genomic sequence (based on strain dl309) to generate recombinant adenoviruses.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and the range of equivalency of the claims are intended to be embraced therein.

What is claimed is:

1. A recombinant adenovirus comprising:
(a) a first nucleotide sequence encoding a first therapeutic transgene inserted into an E1b-19K insertion site; wherein the E1b-19K insertion site is located between the start site of E1b-19K and the start site of E1b-55K; and
(b) a second nucleotide sequence encoding a second therapeutic transgene inserted into an E3 insertion site, wherein the E3 insertion site is located between the stop site of pVIII and the start site of Fiber.

2. The recombinant adenovirus of claim 1, wherein the recombinant adenovirus is a type 5 adenovirus (Ad5).

3. The recombinant adenovirus of claim 1 or 2, wherein the E1b-19K insertion site is located between the start site of E1b-19K and the stop site of E1b-19K.

4. The recombinant adenovirus of any one of claims 1-3, wherein the E1b-19K insertion site comprises a deletion of from about 100 to about 305, about 100 to about 300, about 100 to about 250, about 100 to about 200, about 100 to about 150, about 150 to about 305, about 150 to about 300, about 150 to about 250, or about 150 to about 200 nucleotides adjacent the start site of E1b-19K.

5. The recombinant adenovirus of any one of claims 1-4, wherein the E1b-19K insertion site comprises a deletion of about 200 nucleotides adjacent the start site of E1b-19K.

6. The recombinant adenovirus of any one of claims 1-5, wherein the E1b-19K insertion site comprises a deletion of 202 nucleotides adjacent the start site of E1b-19K.

7. The recombinant adenovirus of any one of claims 1-5, wherein the E1b-19K insertion site comprises a deletion of 203 nucleotides adjacent the start site of E1b-19K.

8. The recombinant adenovirus of any one of claims 1-7, wherein the E1b-19K insertion site comprises a deletion corresponding to nucleotides 1714-1917 of the Ad5 genome (SEQ ID NO: 23).

9. The recombinant adenovirus of any one of claims 1-7, wherein the E1b-19K insertion site comprises a deletion corresponding to nucleotides 1714-1916 of the Ad5 genome (SEQ ID NO: 23).

10. The recombinant adenovirus of any one of claims 1-9, wherein the first therapeutic transgene is inserted between nucleotides corresponding to 1714 and 1917 of the Ad5 genome (SEQ ID NO: 23).

11. The recombinant adenovirus of any one of claims 1-9, wherein the first therapeutic transgene is inserted between nucleotides corresponding to 1714 and 1916 of the Ad5 genome (SEQ ID NO: 23).

12. The recombinant adenovirus of any one of claims 1-11, wherein the first therapeutic transgene is inserted between CTGACCTC (SEQ ID NO: 1) and TCACCAGG (SEQ ID NO: 2).

13. The recombinant adenovirus of any one of claims 1-12, wherein the recombinant adenovirus comprises, in a 5′ to 3′ orientation, CTGACCTC (SEQ ID NO: 1), the first therapeutic transgene, and TCACCAGG (SEQ ID NO: 2).

14. The recombinant adenovirus of any one of claims 1-13, wherein the E3 insertion site comprises a deletion of from about 500 to about 3185, from about 500 to about 3000, from about 500 to about 2500, from about 500 to about 2000, from about 500 to about 1500, from about 500 to about 1000, from about 1000 to about 3185, from about 1000 to about 3000, from about 1000 to about 2500, from about 1000 to about 2000, from about 1000 to about 1500, from about 1500 to about 3185, from about 1500 to about 3000, from about 1500 to about 2000, from about 2000 to about 3185, from about 2000 to about 3000, from about 2000 to about 2500, from about 2500 to about 3185, from about 2500 to about 3000, or from about 3000 to about 3185 nucleotides.

15. The recombinant adenovirus of any one of claims 1-14, wherein the E3 insertion site is located between the stop site of E3-gp19K and the stop site of E3-14.7K.

16. The recombinant adenovirus of any one of claims 1-15, wherein the E3 insertion site is located between the stop site of E3-10.5K and the stop site of E3-14.7K.

17. The recombinant adenovirus of any one of claims 1-16, wherein the E3 insertion site comprises a deletion of from about 500 to about 1551, from about 500 to about 1500, from about 500 to about 1000, from about 1000 to about 1551, from about 1000 to about 1500, or from about 1500 to about 1551 nucleotides adjacent the stop site of E3-10.5K.

18. The recombinant adenovirus of any one of claims 1-17, wherein the E3 insertion site comprises a deletion of about 1050 nucleotides adjacent the stop site of E3-10.5K.

19. The recombinant adenovirus of any one of claims 1-18, wherein the E3 insertion site comprises a deletion of 1063 nucleotides adjacent the stop site of E3-10.5K.

20. The recombinant adenovirus of any one of claims 1-18, wherein the E3 insertion site comprises a deletion of 1064 nucleotides adjacent the stop site of E3-10.5K

21. The recombinant adenovirus of any one of claims 1-18, wherein the E3 insertion site comprises a deletion corresponding to the Ad5 dl309 E3 deletion.

22. The recombinant adenovirus of any one of claims 1-21, wherein the E3 insertion site comprises a deletion corresponding to nucleotides 29773-30836 of the Ad5 genome (SEQ ID NO: 23).

23. The recombinant adenovirus of any one of claims 1-22, wherein the second therapeutic transgene is inserted between nucleotides corresponding to 29773 and 30836 of the Ad5 genome (SEQ ID NO: 23).

24. The recombinant adenovirus of any one of claims 1-23, wherein the second therapeutic transgene is inserted between CAGTATGA (SEQ ID NO: 3) and TAATAAAAAA (SEQ ID NO: 4).

25. The recombinant adenovirus of any one of claims 1-24, wherein the recombinant adenovirus comprises, in a 5′ to 3′ orientation, CAGTATGA (SEQ ID NO: 3), the second therapeutic transgene, and TAATAAAAAA (SEQ ID NO: 4).

26. The recombinant adenovirus of claim 15, wherein the E3 insertion site comprises a deletion of from about 500 to about 1824, from about 500 to about 1500, from about 500 to about 1000, from about 1000 to about 1824, from about 1000 to about 1500, or from about 1500 to about 1824 nucleotides adjacent the stop site of E3-gp19K.

27. The recombinant adenovirus of claim 26, wherein the E3 insertion site comprises a deletion of about 1600 nucleotides adjacent the stop site of E3-gp19K.

28. The recombinant adenovirus of claim 26 or 27, wherein the E3 insertion site comprises a deletion of 1622 nucleotides adjacent the stop site of E3-gp19K.

29. The recombinant adenovirus of any one of claims 26-28, wherein the E3 insertion site comprises a deletion corresponding to nucleotides 29218-30839 of the Ad5 genome (SEQ ID NO: 23).

30. The recombinant adenovirus of any one of claims 26-29, wherein the second therapeutic transgene is inserted between nucleotides corresponding to 29218 and 30839 of the Ad5 genome (SEQ ID NO: 23).

31. The recombinant adenovirus of any one of claims 26-30, wherein the second therapeutic transgene is inserted between TGCCTTAA (SEQ ID NO: 29) and TAAAAAAAAAT (SEQ ID NO: 30).

32. The recombinant adenovirus of any one of claims 26-31, wherein the recombinant adenovirus comprises, in a 5′ to 3′ orientation, TGCCTTAA (SEQ ID NO: 29), the second therapeutic transgene, and TAAAAAAAAAT (SEQ ID NO: 30).

33. A recombinant adenovirus comprising:
(a) a first nucleotide sequence encoding a first therapeutic transgene inserted into an E1b-19k insertion site; and
(b) a second nucleotide sequence encoding a second therapeutic transgene inserted into the E1b-19k insertion site,
wherein the E1b-19k insertion site is located between the start site of E1b-19k and the start site of E1b-55k, and wherein the first nucleotide sequence and the second nucleotide sequence are separated by a first internal ribosome entry site (IRES).

34. The recombinant adenovirus of claim 33, wherein the adenovirus is a type 5 adenovirus (Ad5).

35. The recombinant adenovirus of claim 33 or 34, wherein the E1b-19K insertion site is located between the start site of E1b-19K and the stop site of E1b-19K.

36. The recombinant adenovirus of any one of claims 33-35, wherein the E1b-19K insertion site comprises a deletion of from about 100 to about 305, about 100 to about 300, about 100 to about 250, about 100 to about 200, about 100 to about 150, about 150 to about 305, about 150 to about 300, about 150 to about 250, or about 150 to about 200 nucleotides adjacent the start site of E1b-19K.

37. The recombinant adenovirus of any one of claims 33-36, wherein the E1b-19K insertion site comprises a deletion of about 200 nucleotides adjacent the start site of E1b-19K.

38. The recombinant adenovirus of any one of claims 33-37, wherein the E1b-19K insertion site comprises a deletion of 202 nucleotides adjacent the start site of E1b-19K.

39. The recombinant adenovirus of any one of claims 33-37, wherein the E1b-19K insertion site comprises a deletion of 203 nucleotides adjacent the start site of E1b-19K.

40. The recombinant adenovirus of any one of claims 33-39, wherein the E1b-19K insertion site comprises a deletion corresponding to nucleotides 1714-1917 of the Ad5 genome (SEQ ID NO: 23).

41. The recombinant adenovirus of any one of claims 33-39, wherein the E1b-19K insertion site comprises a deletion corresponding to nucleotides 1714-1916 of the Ad5 genome (SEQ ID NO: 23).

42. The recombinant adenovirus of any one of claims 33-41, wherein the first and second therapeutic transgenes are inserted between nucleotides corresponding to 1714 and 1917 of the Ad5 genome (SEQ ID NO: 23).

43. The recombinant adenovirus of any one of claims 33-41, wherein the first and second therapeutic transgenes are inserted between nucleotides corresponding to 1714 and 1916 of the Ad5 genome (SEQ ID NO: 23).

44. The recombinant adenovirus of any one of claims 33-43, wherein the first and second therapeutic transgenes are inserted between CTGACCTC (SEQ ID NO: 1) and TCACCAGG (SEQ ID NO: 2).

45. The recombinant adenovirus of any one of claims 33-44, wherein the recombinant adenovirus comprises, in a 5′ to 3′ orientation, CTGACCTC (SEQ ID NO: 1), the first therapeutic transgene, the IRES, the second therapeutic transgene, and TCACCAGG (SEQ ID NO: 2).

46. The recombinant adenovirus of any one of claims 33-45, wherein the recombinant adenovirus comprises a third nucleotide sequence encoding a third therapeutic transgene inserted into the E1b-19k insertion site wherein the second nucleotide sequence and the third nucleotide sequence are separated by a second internal ribosome entry site (IRES).

47. The recombinant adenovirus of claim 46, wherein the first, second, and third therapeutic transgenes are inserted between CTGACCTC (SEQ ID NO: 1) and TCACCAGG (SEQ ID NO: 2).

48. The recombinant adenovirus of claim 46 or 47, wherein the recombinant adenovirus comprises, in a 5′ to 3′ orientation, CTGACCTC (SEQ ID NO: 1), the first therapeutic transgene, the first IRES, the second therapeutic transgene, the second IRES, the third therapeutic transgene, and TCACCAGG (SEQ ID NO: 2).

49. The recombinant adenovirus of any of claims 33-48, wherein the recombinant adenovirus further comprises an E3 deletion, wherein the E3 deletion is located between the stop site of pVIII and the start site of Fiber.

50. The recombinant adenovirus of claim 49, wherein the E3 deletion comprises a deletion of from about 500 to about 3185, from about 500 to about 3000, from about 500 to about 2500, from about 500 to about 2000, from about 500 to about 1500, from about 500 to about 1000, from about 1000 to about 3185, from about 1000 to about 3000, from about 1000 to about 2500, from about 1000 to about 2000, from about 1000 to about 1500, from about 1500 to about 3185, from about 1500 to about 3000, from about 1500 to about 2000, from about 2000 to about 3185, from about 2000 to about 3000, from about 2000 to about 2500, from about 2500 to about 3185, from about 2500 to about 3000, or from about 3000 to about 3185 nucleotides.

51. The recombinant adenovirus of claim 49 or 50, wherein the E3 insertion site is located between the stop site of E3-gp19K and the stop site of E3-14.7K.

52. The recombinant adenovirus of any one of claims 49-51, wherein the E3 deletion is located between the stop site of E3-10.5K and the stop site of E3-14.7K and the start site of Fiber.

53. The recombinant adenovirus of any one of claims 49-52, wherein the E3 deletion comprises a deletion of from about 500 to about 1551, from about 500 to about 1500, from about 500 to about 1000, from about 1000 to about 1551, from about 1000 to about 1500, or from about 1500 to about 1551 nucleotides adjacent the stop site of E3-10.5K.

54. The recombinant adenovirus of any one of claims 49-53, wherein the E3 deletion comprises a deletion of about 1050 nucleotides adjacent the stop site of E3-10.5K.

55. The recombinant adenovirus of any one of claims 49-54, wherein the E3 deletion comprises a deletion of 1063 nucleotides adjacent the stop site of E3-10.5K.

56. The recombinant adenovirus of any one of claims 49-54, wherein the E3 deletion comprises a deletion of 1064 nucleotides adjacent the stop site of E3-10.5K.

57. The recombinant adenovirus of any one of claims 49-54, wherein the E3 deletion comprises a deletion corresponding to the Ad5 dl309 E3 deletion.

58. The recombinant adenovirus of any one of claims 49-57, wherein the E3 deletion comprises a deletion corresponding to nucleotides 29773-30836 of the Ad5 genome (SEQ ID NO: 23).

59. The recombinant adenovirus of any one of claims 49-51, wherein the E3 deletion comprises a deletion of from about 500 to about 1824, from about 500 to about 1500, from about 500 to about 1000, from about 1000 to about 1824, from about 1000 to about 1500, or from about 1500 to about 1824 nucleotides adjacent the stop site of E3-gp19K.

60. The recombinant adenovirus of claim 59, wherein the E3 deletion comprises a deletion of about 1600 nucleotides adjacent the stop site of E3-gp19K.

61. The recombinant adenovirus of claim 59 or 60, wherein the E3 deletion comprises a deletion of 1622 nucleotides adjacent the stop site of E3-gp19K.

62. The recombinant adenovirus of any one of claims 59-61, wherein the E3 deletion comprises a deletion corresponding to nucleotides 29218-30839 of the Ad5 genome (SEQ ID NO: 23).

63. The recombinant adenovirus of any of claims 33-45, wherein the recombinant adenovirus comprises a third nucleotide sequence encoding a third therapeutic transgene inserted into an E3 insertion site, wherein the E3 insertion site is located between the stop site of pVIII and the start site of Fiber.

64. The recombinant adenovirus of claim 63, wherein the E3 insertion site comprises a deletion of from about 500 to about 3185, from about 500 to about 3000, from about 500 to about 2500, from about 500 to about 2000, from about 500 to about 1500, from about 500 to about 1000, from about 1000 to about 3185, from about 1000 to about 3000, from about 1000 to about 2500, from about 1000 to about 2000, from about 1000 to about 1500, from about 1500 to about 3185, from about 1500 to about 3000, from about 1500 to about 2000, from about 2000 to about 3185, from about 2000 to about 3000, from about 2000 to about 2500, from about 2500 to about 3185, from about 2500 to about 3000, or from about 3000 to about 3185 nucleotides.

65. The recombinant adenovirus claim 63 or 64, wherein the E3 insertion site is located between the stop site of E3-gp19K and the stop site of E3-14.7K.

66. The recombinant adenovirus of any one of claims 63-65, wherein the E3 insertion site is located between the stop site of E3-10.5K and the stop site of E3-14.7K.

67. The recombinant adenovirus of any one of claims 63-66, wherein the E3 insertion site comprises a deletion of from about 500 to about 1551, from about 500 to about 1500, from about 500 to about 1000, from about 1000 to about 1551, from about 1000 to about 1500, or from about 1500 to about 1551 nucleotides adjacent the stop site of E3-10.5K.

68. The recombinant adenovirus of any one of claims 63-67, wherein the E3 insertion site comprises a deletion of about 1050 nucleotides adjacent the stop site of E3-10.5K.

69. The recombinant adenovirus of any one of claims 63-68, wherein the E3 insertion site comprises a deletion of 1063 nucleotides adjacent the stop site of E3-10.5K.

70. The recombinant adenovirus of any one of claims 63-68, wherein the E3 insertion site comprises a deletion of 1064 nucleotides adjacent the stop site of E3-10.5K.

71. The recombinant adenovirus of any one of claims 63-68, wherein the E3 insertion site comprises a deletion corresponding to the Ad5 dl309 E3 deletion.

72. The recombinant adenovirus of any one of claims 63-71, wherein the E3 insertion site comprises a deletion corresponding to nucleotides 29773-30836 of the Ad5 genome (SEQ ID NO: 23).

73. The recombinant adenovirus of any one of claims 63-72, wherein the third therapeutic transgene is inserted between nucleotides corresponding to 29773 and 30836 of the Ad5 genome (SEQ ID NO: 23).

74. The recombinant adenovirus of any one of claims 63-73, wherein the third therapeutic transgene is inserted between CAGTATGA (SEQ ID NO: 3) and TAATAAAAAA (SEQ ID NO: 4).

75. The recombinant adenovirus of any one of claims 63-74, wherein the recombinant adenovirus comprises, in a 5′ to 3′ orientation, CAGTATGA (SEQ ID NO: 3), the third therapeutic transgene, and TAATAAAAAA (SEQ ID NO: 4).

76. The recombinant adenovirus of any one of claims 63-65, wherein the E3 insertion site comprises a deletion of from about 500 to about 1824, from about 500 to about 1500, from about 500 to about 1000, from about 1000 to about 1824, from about 1000 to about 1500, or from about 1500 to about 1824 nucleotides adjacent the stop site of E3-gp19K.

77. The recombinant adenovirus of claim 76, wherein the E3 insertion site comprises a deletion of about 1600 nucleotides adjacent the stop site of E3-gp19K.

78. The recombinant adenovirus of claim 76 or 77, wherein the E3 insertion site comprises a deletion of 1622 nucleotides adjacent the stop site of E3-gp19K.

79. The recombinant adenovirus of any one of claims 76-78, wherein the E3 insertion site comprises a deletion corresponding to nucleotides 29218-30839 of the Ad5 genome (SEQ ID NO: 23).

80. The recombinant adenovirus of any one of claims 76-79, wherein the third therapeutic transgene is inserted between nucleotides corresponding to 29218 and 30839 of the Ad5 genome (SEQ ID NO: 23).

81. The recombinant adenovirus of any one of claims 76-80, wherein the third therapeutic transgene is inserted between TGCCTTAA (SEQ ID NO: 29) and TAAAAAAAAAT (SEQ ID NO: 30).

82. The recombinant adenovirus of any one of claims 76-81, wherein the recombinant adenovirus comprises, in a 5′ to 3′ orientation, TGCCTTAA (SEQ ID NO: 29), the third therapeutic transgene, and TAAAAAAAAAT (SEQ ID NO: 30).

83. The recombinant adenovirus of any one of claims 33-82, wherein the IRES is selected from the group consisting the encephalomyocarditis virus IRES, the foot-and-mouth disease virus IRES, and the poliovirus IRES.

84. The recombinant adenovirus of any of claims 1-83, wherein the recombinant adenovirus further comprises an E4 deletion, wherein the E4 deletion is located between the start site of E4-ORF6/7 and right inverted terminal repeat (ITR).

85. The recombinant adenovirus of claim 84, wherein the E4 deletion is located between the start site of E4-ORF6/7 and the start site of E4-ORF1.

86. The recombinant adenovirus of claim 84 or 85, wherein the E4 deletion comprises a deletion of from about 500 to about 2500, from about 500 to about 2000, from about 500 to about 1500, from about 500 to about 1000, from about 1000 to about 2500, from about 1000 to about 2000, from about 1000 to about 1500, from about 1500 to about 2500, from about 1500 to about 2000, or from about 2000 to about 2500 nucleotides.

87. The recombinant adenovirus of any one of claims 84-86, wherein the E4 deletion comprises a deletion of from about 250 to about 1500, from about 250 to about 1250, from about 250 to about 1000, from about 250 to about 750, from about 250 to about 500, from 500 to about 1500, from about 500 to about 1250, from about 500 to about 1000, from about 500 to about 750, from 750 to about 1500, from about 750 to about 1250, from about 750 to about 1000, from about 1000 to about 1500, from about 1000 to about 1250, or from about 1250 to about 1500 nucleotides adjacent the start site of E4-ORF6/7.

88. The recombinant adenovirus of any one of claims 84-87, wherein the E4 deletion comprises a deletion of about 1450 nucleotides adjacent the start site of E4-ORF6/7.

89. The recombinant adenovirus of any one of claims 84-88, wherein the E4 deletion comprises a deletion of 1449 nucleotides adjacent the start site of E4-ORF6/7.

90. The recombinant adenovirus of any one of claims 84-89, wherein the E4 deletion comprises a deletion corresponding to nucleotides 34078-35526 of the Ad5 genome (SEQ ID NO: 23).

91. The recombinant adenovirus of any one of claims 1-90, wherein the first and/or second therapeutic transgenes are not operably linked to an exogenous promoter sequence.

92. The recombinant adenovirus of any one of claims 46-90, wherein the first, second, and/or third therapeutic transgenes are not operably linked to an exogenous promoter sequence.

93. The recombinant adenovirus of any one of claims 1-90, wherein none of the therapeutic transgenes are operably linked to an exogenous promoter sequence.

94. The recombinant adenovirus of any one of claims 1-93, wherein the combined size of the first and second therapeutic transgenes comprises from about 500 to about 5000, from about 500 to about 4000, from about 500 to about 3000, from about 500 to about 2000, from about 500 to about 1000, from about 1000 to about 5000, from about 1000 to about 4000, from about 1000 to about 3000, from about 1000 to about 2000, from about 2000 to about 5000, from about 2000 to about 4000, from about 2000 to about 3000, from about 3000 to about 5000, from about 3000 to about 4000, or from about 4000 to about 5000 nucleotides.

95. The recombinant adenovirus of any one of claims 1-93, wherein the combined size of the first and second therapeutic transgenes comprises from about 500 to about 7000, from about 500 to about 6000, from about 500 to about 5000, from about 500 to about 4000, from about 500 to about 3000, from about 500 to about 2000, from about 500 to about 1000, from about 1000 to about 7000, from about 1000 to about 6000, from about 1000 to about 5000, from about 1000 to about 4000, from about 1000 to about 3000, from about 1000 to about 2000, from about 2000 to about 7000, from about 2000 to about 6000, from about 2000 to about 5000, from about 2000 to about 4000, from about 2000 to about 3000, from about 3000 to about 7000, from about 3000 to about 6000, from about 3000 to about 5000, from about 3000 to about 4000, from about 4000 to about 7000, from about 4000 to about 6000, from about 4000 to about 5000 nucleotides, from about 5000 to about 7000, from about 5000 to about 6000, or from about 6000 to about 7000 nucleotides.

96. The recombinant adenovirus of any one of claims 46-93, wherein the combined size of the first, second, and third therapeutic transgenes comprises from about 500 to about 5000, from about 500 to about 4000, from about 500 to about 3000, from about 500 to about 2000, from about 500 to about 1000, from about 1000 to about 5000, from about 1000 to about 4000, from about 1000 to about 3000, from about 1000 to about 2000, from about 2000 to about 5000, from about 2000 to about 4000, from about 2000 to about 3000, from about 3000 to about 5000, from about 3000 to about 4000, or from about 4000 to about 5000 nucleotides.

97. The recombinant adenovirus of any one of claims 46-93, wherein the combined size of the first, second, and third therapeutic transgenes comprises from about 500 to about 7000, from about 500 to about 6000, from about 500 to about 5000, from about 500 to about 4000, from about 500 to about 3000, from about 500 to about 2000, from about 500 to about 1000, from about 1000 to about 7000, from about 1000 to about 6000, from about 1000 to about 5000, from about 1000 to about 4000, from about 1000 to about 3000, from about 1000 to about 2000, from about 2000 to about 7000, from about 2000 to about 6000, from about 2000 to about 5000, from about 2000 to about 4000, from about 2000 to about 3000, from about 3000 to about 7000, from about 3000 to about 6000, from about 3000 to about 5000, from about 3000 to about 4000, from about 4000 to about 7000, from about 4000 to about 6000, from about 4000 to about 5000 nucleotides, from about 5000 to about 7000, from about 5000 to about 6000, or from about 6000 to about 7000 nucleotides.

98. The recombinant adenovirus of any one of claims 1-97, wherein the combined size of each of the therapeutic transgenes comprises from about 500 to about 5000, from about 500 to about 4000, from about 500 to about 3000, from about 500 to about 2000, from about 500 to about 1000, from about 1000 to about 5000, from about 1000 to about 4000, from about 1000 to about 3000, from about 1000 to about 2000, from about 2000 to about 5000, from about 2000 to about 4000, from about 2000 to about 3000, from about 3000 to about 5000, from about 3000 to about 4000, or from about 4000 to about 5000 nucleotides.

99. The recombinant adenovirus of any one of claims 1-97, wherein the combined size of each of the therapeutic transgenes comprises from about 500 to about 7000, from about 500 to about 6000, from about 500 to about 5000, from about 500 to about 4000, from about 500 to about 3000, from about 500 to about 2000, from about 500 to about 1000, from about 1000 to about 7000, from about 1000 to about 6000, from about 1000 to about 5000, from about 1000 to about 4000, from about 1000 to about 3000, from about 1000 to about 2000, from about 2000 to about 7000, from about 2000 to about 6000, from about 2000 to about 5000, from about 2000 to about 4000, from about 2000 to about 3000, from about 3000 to about 7000, from about 3000 to about 6000, from about 3000 to about 5000, from about 3000 to about 4000, from about 4000 to about 7000, from about 4000 to about 6000, from about 4000 to about 5000 nucleotides, from about 5000 to about 7000, from about 5000 to about 6000, or from about 6000 to about 7000 nucleotides.

100. The recombinant adenovirus of any one of claims 1-99, wherein the combined size of the first and second therapeutic transgenes comprises at least from about 500 to about 5000, from about 500 to about 4000, from about 500 to about 3000, from about 500 to about 2000, from about 500 to about 1000, from about 1000 to about 5000, from about 1000 to about 4000, from about 1000 to about 3000, from about 1000 to about 2000, from about 2000 to about 5000, from about 2000 to about 4000, from about 2000 to about 3000, from about 3000 to about 5000, from about 3000 to about 4000, or from about 4000 to about 5000 nucleotides.

101. The recombinant adenovirus of any one of claims 1-99, wherein the combined size of the first and second therapeutic transgenes comprises at least from about 500 to about 7000, from about 500 to about 6000, from about 500 to about 5000, from about 500 to about 4000, from about 500 to about 3000, from about 500 to about 2000, from about 500 to about 1000, from about 1000 to about 7000, from about 1000 to about 6000, from about 1000 to about 5000, from about 1000 to about 4000, from about 1000 to about 3000, from about 1000 to about 2000, from about 2000 to about 7000, from about 2000 to about 6000, from about 2000 to about 5000, from about 2000 to about 4000, from about 2000 to about 3000, from about 3000 to about 7000, from about 3000 to about 6000, from about 3000 to about 5000, from about 3000 to about 4000, from about 4000 to about 7000, from about 4000 to about 6000, from about 4000 to about 5000 nucleotides, from about 5000 to about 7000, from about 5000 to about 6000, or from about 6000 to about 7000 nucleotides.

102. The recombinant adenovirus of any one of claims 46-99, wherein the combined size of the first, second, and third therapeutic transgenes comprises at least from about 500 to about 5000, from about 500 to about 4000, from about 500 to about 3000, from about 500 to about 2000, from about 500 to about 1000, from about 1000 to about 5000, from about 1000 to about 4000, from about 1000 to about 3000, from about 1000 to about 2000, from about 2000 to about 5000, from about 2000 to about 4000, from about 2000 to about 3000, from about 3000 to about 5000, from about 3000 to about 4000, or from about 4000 to about 5000 nucleotides.

103. The recombinant adenovirus of any one of claims 46-99, wherein the combined size of the first, second, and third therapeutic transgenes comprises at least from about 500 to about 7000, from about 500 to about 6000, from about 500 to about 5000, from about 500 to about 4000, from about 500 to about 3000, from about 500 to about 2000, from about 500 to about 1000, from about 1000 to about 7000, from about 1000 to about 6000, from about 1000 to about 5000, from about 1000 to about 4000, from about 1000 to about 3000, from about 1000 to about 2000, from about 2000 to about 7000, from about 2000 to about 6000, from about 2000 to about 5000, from about 2000 to about 4000, from about 2000 to about 3000, from about 3000 to about 7000, from about 3000 to about 6000, from about 3000 to about 5000, from about 3000 to about 4000, from about 4000 to about 7000, from about 4000 to about 6000, from about 4000 to about 5000 nucleotides, from about 5000 to about 7000, from about 5000 to about 6000, or from about 6000 to about 7000 nucleotides.

104. The recombinant adenovirus of any one of claims 1-99, wherein the combined size of each of the therapeutic transgenes comprises at least from about 500 to about 5000, from about 500 to about 4000, from about 500 to about 3000, from about 500 to about 2000, from about 500 to about 1000, from about 1000 to about 5000, from about 1000 to about 4000, from about 1000 to about 3000, from about 1000 to about 2000, from about 2000 to about 5000, from about 2000 to about 4000, from about 2000 to about 3000, from about 3000 to about 5000, from about 3000 to about 4000, or from about 4000 to about 5000 nucleotides.

105. The recombinant adenovirus of any one of claims 1-99, wherein the combined size of each of the therapeutic transgenes comprises at least from about 500 to about 7000, from about 500 to about 6000, from about 500 to about 5000, from about 500 to about 4000, from about 500 to about 3000, from about 500 to about 2000, from about 500 to about 1000, from about 1000 to about 7000, from about 1000 to about 6000, from about 1000 to about 5000, from about 1000 to about 4000, from about 1000 to about 3000, from about 1000 to about 2000, from about 2000 to about 7000, from about 2000 to about 6000, from about 2000 to about 5000, from about 2000 to about 4000, from about 2000 to about 3000, from about 3000 to about 7000, from about 3000 to about 6000, from about 3000 to about 5000, from about 3000 to about 4000, from about 4000 to about 7000, from about 4000 to about 6000, from about 4000 to about 5000 nucleotides, from about 5000 to about 7000, from about 5000 to about 6000, or from about 6000 to about 7000 nucleotides.

106. The recombinant adenovirus of any one of claims 1-105, wherein the combined size of the first and second therapeutic transgenes comprises at least about 500, about 1000, about 2000, about 3000, about 4000, or about 5000 nucleotides.

107. The recombinant adenovirus of any one of claims 1-105, wherein the combined size of the first and second therapeutic transgenes comprises at least about 500, about 1000, about 2000, about 3000, about 4000, about 5000, about 6000, or about 7000 nucleotides.

108. The recombinant adenovirus of any one of claims 46-105, wherein the combined size of the first, second, and third therapeutic transgenes comprises at least about 500, about 1000, about 2000, about 3000, about 4000, or about 5000 nucleotides.

109. The recombinant adenovirus of any one of claims 46-105, wherein the combined size of the first, second, and third therapeutic transgenes comprises at least about 500, about 1000, about 2000, about 3000, about 4000, about 5000, about 6000, or about 7000 nucleotides.

110. The recombinant adenovirus of any one of claims 1-109, wherein the combined size of each of the therapeutic transgenes comprises at least about 500, about 1000, about 2000, about 3000, about 4000, or about 5000 nucleotides.

111. The recombinant adenovirus of any one of claims 1-109, wherein the combined size of each of the therapeutic transgenes comprises at least about 500, about 1000, about 2000, about 3000, about 4000, about 5000, about 6000, or about 7000 nucleotides.

112. The recombinant adenovirus of any one of claims 1-111, wherein the combined size of the first and second therapeutic transgenes comprises about 1650 nucleotides.

113. The recombinant adenovirus of any one of claims 1-111, wherein the combined size of the first and second therapeutic transgenes comprises about 3100 nucleotides.

114. The recombinant adenovirus of any one of claims 46-111, wherein the combined size of the first, second, and third therapeutic transgenes comprises about 1650 nucleotides.

115. The recombinant adenovirus of any one of claims 46-111, wherein the combined size of the first, second, and third therapeutic transgenes comprises about 3100 nucleotides.

116. The recombinant adenovirus of any one of claims 1-115, wherein the combined size of each of the therapeutic transgenes comprises about 1650 nucleotides.

117. The recombinant adenovirus of any one of claims 1-115, wherein the combined size of each of the therapeutic transgenes comprises about 3100 nucleotides.

118. The recombinant adenovirus of any one of claims 1-117, wherein the first and/or second therapeutic transgene encodes a therapeutic polypeptide selected from the group consisting of CD80, CD137L, IL-23A/p19, endostatin, angiostatin, ICAM-1, and a TGF-β trap.

119. The recombinant adenovirus of any one of claims 46-117, wherein the first, second and/or third therapeutic transgene encodes a therapeutic polypeptide selected from the group consisting of CD80, CD137L, IL-23A/p19, endostatin, angiostatin, ICAM-1, and a TGF-β trap.

120. The recombinant adenovirus of any one of claims 1-117, wherein any one of the therapeutic transgenes encode a therapeutic polypeptide selected from the group consisting of CD80, CD137L, IL-23A/p19, endostatin, angiostatin, ICAM-1, and a TGF-β trap.

121. The recombinant adenovirus of any one of claims 1-117, wherein the first and/or second therapeutic transgene encodes a therapeutic polypeptide selected from the group consisting of CD80, CD137L, IL-23A/p19, endostatin, angiostatin, ICAM-1, a TGF-β trap, TGF-β, CD19, CD20, IL-1, IL-3, IL-4, IL-5, IL-6, IL-8, IL-9, CD154, CD86, BORIS/CTCFL, FGF, IL-24, MAGE, NY-ESO-1, acetylcholine, interferon-gamma, DKK1/Wnt, p53, thymidine kinase, an anti-PD-1 antibody heavy chain or light chain, and an anti-PD-L1 antibody heavy chain or light chain.

122. The recombinant adenovirus of any one of claims 46-117, wherein the first, second and/or third therapeutic transgene encodes a therapeutic polypeptide selected from the group consisting of CD80, CD137L, IL-23A/p19, endostatin, angiostatin, ICAM-1, a TGF-β trap, TGF-β, CD19, CD20, IL-1, IL-3, IL-4, IL-5, IL-6, IL-8, IL-9, CD154, CD86, BORIS/CTCFL, FGF, IL-24, MAGE, NY-ESO-1, acetylcholine, interferon-gamma, DKK1/Wnt, p53, thymidine kinase, an anti-PD-1 antibody heavy chain or light chain, and an anti-PD-L1 antibody heavy chain or light chain.

123. The recombinant adenovirus of any one of claims 1-117, wherein any one of the therapeutic transgenes encode a therapeutic polypeptide selected from the group consisting of CD80, CD137L, IL-23A/p19, endostatin, angiostatin, ICAM-1, a TGF-β trap, TGF-β, CD19, CD20, IL-1, IL-3, IL-4, IL-5, IL-6, IL-8, IL-9, CD154, CD86, BORIS/CTCFL, FGF, IL-24, MAGE, NY-ESO-1, acetylcholine, interferon-gamma, DKK1/Wnt, p53, thymidine kinase, an anti-PD-1 antibody heavy chain or light chain, and an anti-PD-L1 antibody heavy chain or light chain.

124. The recombinant adenovirus of any one of claims 1-117, wherein the first and/or second therapeutic transgene encodes a therapeutic polypeptide selected from the group consisting of CD80, CD137L, IL-23, IL-23A/p19, IL-27, IL-27A/p28, IL-27B/EBI3, endostatin, angiostatin, ICAM-1, a TGF-β trap, TGF-β, CD19, CD20, IL-1, IL-3, IL-4, IL-5, IL-6, IL-8, IL-9, CD154, CD86, BORIS/CTCFL, FGF, IL-24, MAGE, NY-ESO-1, acetylcholine, interferon-gamma, DKK1/Wnt, p53, thymidine kinase, an anti-PD-1 antibody heavy chain or light chain, and an anti-PD-L1 antibody heavy chain or light chain.

125. The recombinant adenovirus of any one of claims 46-117, wherein the first, second and/or third therapeutic transgene encodes a therapeutic polypeptide selected from the group consisting of CD80, CD137L, IL-23, IL-23A/p19, IL-27, IL-27A/p28, IL-27B/EBI3, endostatin, angiostatin, ICAM-1, a TGF-β trap, TGF-β, CD19, CD20, IL-1, IL-3, IL-4, IL-5, IL-6, IL-8, IL-9, CD154, CD86, BORIS/CTCFL, FGF, IL-24, MAGE, NY-ESO-1, acetylcholine, interferon-gamma, DKK1/Wnt, p53, thymidine kinase, an anti-PD-1 antibody heavy chain or light chain, and an anti-PD-L1 antibody heavy chain or light chain.

126. The recombinant adenovirus of any one of claims 1-117, wherein any one of the therapeutic transgenes encode a therapeutic polypeptide selected from the group consisting of CD80, CD137L, IL-23, IL-23A/p19, IL-27, IL-27A/p28, IL-27B/EBI3, endostatin, angiostatin, ICAM-1, a TGF-β trap, TGF-β, CD19, CD20, IL-1, IL-3, IL-4, IL-5, IL-6, IL-8, IL-9, CD154, CD86, BORIS/CTCFL, FGF, IL-24, MAGE, NY-ESO-1, acetylcholine, interferon-gamma, DKK1/Wnt, p53, thymidine kinase, an anti-PD-1 antibody heavy chain or light chain, and an anti-PD-L1 antibody heavy chain or light chain.

127. The recombinant adenovirus of any one of claims 1-126, wherein the first and second therapeutic transgene encode a first and second subunit, respectively, of a heterodimeric cytokine.

128. The recombinant adenovirus of any one of claim 1-126, wherein the first and/or second therapeutic transgenes are selected from the group consisting of CD80 and CD137L.

129. The recombinant adenovirus of any one of claim 46-126, wherein the first, second and/or third therapeutic transgenes are selected from the group consisting of CD80, CD137L, and ICAM-1.

130. The recombinant adenovirus of claim 127 or 128, wherein the first therapeutic transgene encodes CD80.

131. The recombinant adenovirus of any one of claims 127-130, wherein the second therapeutic transgene encodes CD137L.

132. The recombinant adenovirus of any one of claims 128-131, wherein the third therapeutic transgene encodes ICAM-1.

133. The recombinant adenovirus of any one of claims 128-132, wherein the recombinant adenovirus comprises a nucleotide sequence encoding an amino acid sequence that is encoded by SEQ ID NO: 5.

134. The recombinant adenovirus of any one of claims 128-133, wherein the recombinant adenovirus comprises the nucleotide sequence of SEQ ID NO: 6.

135. The recombinant adenovirus of any one of claims 128-134, wherein the recombinant adenovirus comprises a nucleotide sequence encoding an amino acid sequence that is encoded by SEQ ID NO: 7.

136. The recombinant adenovirus of any one of claims 128-135, wherein the recombinant adenovirus comprises the nucleotide sequence of SEQ ID NO: 8.

137. The recombinant adenovirus of any one of claims 128-136, wherein the recombinant adenovirus comprises the nucleotide sequence of SEQ ID NO: 27.

138. The recombinant adenovirus of any one of claims 128-137, wherein the recombinant adenovirus comprises a nucleotide sequence encoding an amino acid sequence that is encoded by SEQ ID NO: 32.

139. The recombinant adenovirus of any one of claims 128-138, wherein the recombinant adenovirus comprises the nucleotide sequence of SEQ ID NO: 31 or SEQ ID NO: 9.

140. The recombinant adenovirus of any one of claims 128-137, wherein the recombinant adenovirus comprises the nucleotide sequence of SEQ ID NO: 31.

141. The recombinant adenovirus of any one of claims 128-137, wherein the recombinant adenovirus comprises the nucleotide sequence of SEQ ID NO: 22.

142. The recombinant adenovirus of any one of claims 1-127, wherein the first and/or second therapeutic transgenes are selected from the group consisting of IL-27A/p28 and IL-27B/EBI3.

143. The recombinant adenovirus of claim 142, wherein the first therapeutic transgene encodes IL-27A/p28.

144. The recombinant adenovirus of claim 142 or 143, wherein the second therapeutic transgene encodes IL-27B/EBI3.

145. The recombinant adenovirus of any one of claims 1-126, wherein the first and/or second therapeutic transgenes are selected from the group consisting of endostatin and angiostatin.

146. The recombinant adenovirus of claim 145, wherein the first therapeutic transgene encodes endostatin.

147. The recombinant adenovirus of claim 145 or 146, wherein the second therapeutic transgene encodes angiostatin

148. The recombinant adenovirus of any one of claims 145-147, wherein the recombinant adenovirus comprises a nucleotide sequence encoding the amino acid sequence of SEQ ID NO: 37 or SEQ ID NO: 38.

149. The recombinant adenovirus of any one of claims 145-148, wherein the recombinant adenovirus comprises a nucleotide sequence encoding the amino acid sequence of SEQ ID NO: 39, SEQ ID NO: 40, SEQ ID NO: 41, SEQ ID NO: 42, SEQ ID NO: 43 or SEQ ID NO: 44.

150. The recombinant adenovirus of any one of claims 145-149, wherein the recombinant adenovirus comprises the nucleotide sequence of SEQ ID NO: 11.

151. The recombinant adenovirus of any one of claims 1-150, wherein the recombinant adenovirus further comprises a deletion of a Pea3 binding site, or a functional fragment thereof.

152. The recombinant adenovirus of claim 151, wherein the recombinant adenovirus comprises a deletion of nucleotides corresponding to about −300 to about −250 upstream of the initiation site of E1a.

153. The recombinant adenovirus of claim 151 or 152, wherein the recombinant adenovirus comprises a deletion of nucleotides corresponding to −305 to −255 upstream of the initiation site of E1a.

154. The recombinant adenovirus of claim 151 or 152, wherein the recombinant adenovirus comprises a deletion of nucleotides corresponding to −304 to −255 upstream of the initiation site of E1a.

155. A recombinant adenovirus comprising SEQ ID NO: 14, or a sequence having 80%, 85%, 86%, 87%, 88%, 89%, 90%, 91%, 92%, 93%, 94%, 95%, 96%, 97%, 98%, or 99% sequence identity to SEQ ID NO: 14.

156. The recombinant adenovirus of any one of claims 1-155, wherein the recombinant adenovirus selectively replicates in a hyperproliferative cell.

157. The recombinant adenovirus of any one of claims 1-156, wherein the recombinant adenovirus selectively expresses the first and/or the second therapeutic transgene in a hyperproliferative cell.

158. The recombinant adenovirus of any one of claims 46-157, wherein the recombinant adenovirus selectively expresses the first, second, and/or third therapeutic transgene in a hyperproliferative cell.

159. The recombinant adenovirus of any one of claims 156-158, wherein the hyperproliferative cell is a cancer cell.

160. The recombinant adenovirus of any one of claims 1-159, wherein the recombinant adenovirus is an oncolytic virus.

161. A pharmaceutical composition comprising the recombinant adenovirus of any one of claims 1-160 and at least one pharmaceutically acceptable carrier or diluent.

162. A method of expressing two therapeutic transgenes in a target cell comprising exposing the cell to an effective amount of the recombinant adenovirus of any one of claims 1-160 to express the two therapeutic transgenes.

163. A method of expressing three therapeutic transgenes in a target cell comprising exposing the cell to an effective amount of the recombinant adenovirus of any one of claims 46-160 to express the two therapeutic transgenes.

164. A method of inhibiting proliferation of a tumor cell comprising exposing the cell to an effective amount of the recombinant adenovirus of any one of claims 1-160 to inhibit proliferation of the tumor cell.

165. A method of inhibiting tumor growth in a subject in need thereof, the method comprising administering to the subject to an effective amount of the recombinant adenovirus of any one of claims 1-160 to inhibit growth of the tumor.

166. A method of treating cancer in a subject in need thereof, the method comprising administering to the subject an effective amount of the recombinant adenovirus of any one of claims 1-160 to treat the cancer in the subject.

167. The method of claim 166, wherein the cancer is selected from the group consisting of melanoma, squamous cell carcinoma of the skin, basal cell carcinoma, head and neck cancer, breast cancer, anal cancer, cervical cancer, non-small cell lung cancer, mesothelioma, small cell lung cancer, renal cell carcinoma, prostate cancer, gastroesophageal cancer, colorectal cancer, testicular cancer, bladder cancer, ovarian cancer, hepatocellular carcinoma, cholangiocarcinoma, brain cancer, endometrial cancer, neuroendocrine cancer, merkel cell carcinoma, gastrointestinal stromal tumors, a sarcoma, and pancreatic cancer.

168. The method of claims 165-167, wherein the recombinant adenovirus is administered in combination with one or more therapies selected from the group consisting of surgery, radiation, chemotherapy, immunotherapy, hormone therapy, and virotherapy.

169. The method of any one of claims 162-168, wherein the effective amount of the recombinant adenovirus is 10²-10¹⁵ plaque forming units (pfus).

170. The method of any one of claims 165-169, wherein the subject is a human.

171. The method of claim 170, wherein the subject is a pediatric human.

172. The method of any one of claims 165-171, wherein the method further comprises measuring an immune response to an antigen in the subject.

173. The method of any one of claims 165-172, wherein the effective amount of the recombinant virus is identified by measuring an immune response to an antigen in the subject.

174. The method of claim 172 or 173, wherein the immune response to the antigen is measured by injecting the subject with the antigen at an injection site on the skin of the subject and measuring the size of an induration at the injection site.


SEQUENCE LISTING (44 sequences)

SEQ ID NO: 1 - DNA, 8 bp, Adenovirus type 5
ctgacctc

SEQ ID NO: 2 - DNA, 8 bp, Adenovirus type 5
tcaccagg

SEQ ID NO: 3 - DNA, 8 bp, Adenovirus type 5
cagtatga

SEQ ID NO: 4 - DNA, 10 bp, Adenovirus type 5
taataaaaaa

SEQ ID NO: 5 - DNA, 866 bp, Homo sapiens
atgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttgccccaagatgcagagagagaaggaggaatgagagattgagaagggaaagtgtacgccctgtata

SEQ ID NO: 6 - DNA, 895 bp, Artificial Sequence
  human CD80 cloned into modified E1b-19k region with flanking adenoviral sequences
ctgacctcgtcgacatgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttgccccaagatgcagagagagaaggaggaatgagagattgagaagggaaagtgtacgccctgtataactcgagtcaccagg

SEQ ID NO: 7 - DNA, 765 bp, Homo sapiens
atggaatacgcctctgacgcttcactggaccccgaagccccgtggcctcctgcacctcgcgctcgcgcctgccgcgtactgccttgggccctggtcgcggggctgctgctcctgctcctgctcgctgctgcatgcgctgtatttcttgcatgcccatgggctgtgtctggggctcgcgcatcacctggctccgcggccagcccgagactccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaataa

SEQ ID NO: 8 - DNA, 986 bp, Artificial Sequence
  human CD137L cloned into modified E3 region with flanking adenoviral sequences
cagtatgattaaatgagacatggaccagcacacacttgatgtggaggataccgcggatgccagacatccagcaggtacttcgtgcccctcggatgcggcgctcctcagagataccgggctcctcgcggacgctgcgctcctctcagatactgtgcgccccacaaatgccgcgctccccacggatgctgcctaccctgcggttaatgttcgggatcgcgaggccgcgtggccgcctgcactgaacttctgttcccgccacccaaagctctatggcctagtcgctttggttttgctgcttctgatcgccgcctgtgttcctatcttcacccgcaccgagcctcggccagcgctcacaatcaccacctcgcccaacctgggtacccgagagaataatgcagaccaggtcacccctgtttcccacattggctgccccaacactacacaacagggctctcctgtgttcgccaagctactggctaaaaaccaagcatcgttgtgcaatacaactctgaactggcacagccaagatggagctgggagctcatacctatctcaaggtctgaggtacgaagaagacaaaaaggagttggtggtagacagtcccgggctctactacgtatttttggaactgaagctcagtccaacattcacaaacacaggccacaaggtgcagggctgggtctctcttgttttgcaagcaaagcctcaggtagatgactttgacaacttggccctgacagtggaactgttcccttgctccatggagaacaagttagtggaccgttcctggagtcaactgttgctcctgaaggctggccaccgcctcagtgtgggtctgagggcttatctgcatggagcccaggatgcatacagagactgggagctgtcttatcccaacaccaccagctttggactctttcttgtgaaacccgacaacccatgggaatgaggtctcaaagatcttattccctttaactaataaaaaa

SEQ ID NO: 9 - DNA, 4271 bp, Artificial Sequence
  human CD80 - EMCV IRES - CD137L - FMDV IRES - ICAM cloned into modified E1b-19k region with flanking adenoviral sequences
ctgacctcgtcgacatgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttgccccaagatgcagagagagaaggaggaatgagagattgagaagggaaagtgtacgccctgtataataacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggaatacgcctctgacgcttcactggaccccgaagccccgtggcctcctgcacctcgcgctcgcgcctgccgcgtactgccttgggccctggtcgcggggctgctgctcctgctcctgctcgctgctgcatgcgctgtatttcttgcatgcccatgggctgtgtctggggctcgcgcatcacctggctccgcggccagcccgagactccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaataaggtttccacaactgataaaactcgtgcaacttgaaactccgcctggtctttccaggtctagaggggttacactttgtactgtgctcgactccacgcccggtccactggcgggtgttagtagcagcactgttgtttcgtagcggagcatggtggccgtgggaactcctccttggtgacaagggcccacggggccgaaagccacgtccagacggacccaccatgtgtgcaaccccagcacggcaacttttactgcgaacaccaccttaaggtgacactggtactggtactcggtcactggtgacaggctaaggatgcccttcaggtaccccgaggtaacacgggacactcgggatctgagaaggggattgggacttctttaaaagtgcccagtttaaaaagcttctacgcctgaataggcgaccggaggccggcgcctttccattacccactactaaatccatggctcccagcagcccccggcccgcgctgcccgcactcctggtcctgctcggggctctgttcccaggacctggcaatgcccagacatctgtgtccccctcaaaagtcatcctgccccggggaggctccgtgctggtgacatgcagcacctcctgtgaccagcccaagttgttgggcatagagaccccgttgcctaaaaaggagttgctcctgcctgggaacaaccggaaggtgtatgaactgagcaatgtgcaagaagatagccaaccaatgtgctattcaaactgccctgatgggcagtcaacagctaaaaccttcctcaccgtgtactggactccagaacgggtggaactggcacccctcccctcttggcagccagtgggcaagaaccttaccctacgctgccaggtggagggtggggcaccccgggccaacctcaccgtggtgctgctccgtggggagaaggagctgaaacgggagccagctgtgggggagcccgctgaggtcacgaccacggtgctggtgaggagagatcaccatggagccaatttctcgtgccgcactgaactggacctgcggccccaagggctggagctgtttgagaacacctcggccccctaccagctccagacctttgtcctgccagcgactcccccacaacttgtcagcccccgggtcctagaggtggacacgcaggggaccgtggtctgttccctggacgggctgttcccagtctcggaggcccaggtccacctggcactgggggaccagaggttgaaccccacagtcacctatggcaacgactccttctcggccaaggcctcagtcagtgtgaccgcagaggacgagggcacccagcggctgacgtgtgcagtaatactggggaaccagagccaggagacactgcagacagtgaccatctacagctttccggcgcccaacgtgattctgacgaagccagaggtctcagaagggaccgaggtgacagtgaagtgtgaggcccaccctagagccaaggtgacgctgaatggggttccagcccagccactgggcccgagggcccagctcctgctgaaggccaccccagaggacaacgggcgcagcttctcctgctctgcaaccctggaggtggccggccagcttatacacaagaaccagacccgggagcttcgtgtcctgtatggcccccgactggacgagagggattgtccgggaaactggacgtggccagaaaattcccagcagactccaatgtgccaggcttgggggaacccattgcccgagctcaagtgtctaaaggatggcactttcccactgcccatcggggaatcagtgactgtcactcgagatcttgagggcacctacctctgtcgggccaggagcactcaaggggaggtcacccgcaaggtgaccgtgaatgtgctctccccccggtatgagattgtcatcatcactgtggtagcagccgcagtcataatgggcactgcaggcctcagcacgtacctctataaccgccagcggaagatcaagaaatacagactacaacaggcccaaaaagggacccccatgaaaccgaacacacaagccacgcctccctgactcgagtcaccagg

SEQ ID NO: 10 - DNA, 990 bp, Homo sapiens
atgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttagtaa

SEQ ID NO: 11 - DNA, 2669 bp, Artificial Sequence
  human endostatin - IRES - angiostatin cloned into modified E1b-19k region with flanking adenoviral sequences
ctgacctcgtcgacatggctccctacccctgtggctgccacatcctgctgctgctcttctgctgcctggcggctgcccgggccagctcctacgtgcacctgcggccggcgcgacccacaagcccacccgcccacagccaccgcgacttccagccggtgctccacctggttgcgctcaacagccccctgtcaggcggcatgcggggcatccgcggggccgacttccagtgcttccagcaggcgcgggccgtggggctggcgggcaccttccgcgccttcctgtcctcgcgcctgcaggacctgtacagcatcgtgcgccgtgccgaccgcgcagccgtgcccatcgtcaacctcaaggacgagctgctgtttcccagctgggaggctctgttctcaggctctgagggtccgctgaagcccggggcacgcatcttctcctttgacggcaaggacgtcctgaggcaccccacctggccccagaagagcgtgtggcatggctcggaccccaacgggcgcaggctgaccgagagctactgtgagacgtggcggacggaggctccctcggccacgggccaggcctcctcgctgctggggggcaggctcctggggcagagtgccgcgagctgccatcacgcctacatcgtgctctgcattgagaacagcttcatgactgcctccaagtagtaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggaacataaggaagtggttcttctacttcttttatttctgaaatcaggtcaaggaaaagtgtatctctcagagtgcaagactgggaatggaaagaactacagagggacgatgtccaaaacaaaaaatggcatcacctgtcaaaaatggagttccacttctccccacagacctagattctcacctgctacacacccctcagagggactggaggagaactactgcaggaatccagacaacgatccgcaggggccctggtgctatactactgatccagaaaagagatatgactactgcgacattcttgagtgtgaagaggaatgtatgcattgcagtggagaaaactatgacggcaaaatttccaagaccatgtctggactggaatgccaggcctgggactctcagagcccacacgctcatggatacattccttccaaatttccaaacaagaacctgaagaagaattactgtcgtaaccccgatagggagctgcggccttggtgtttcaccaccgaccccaacaagcgctgggaactttgtgacatcccccgctgcacaacacctccaccatcttctggtcccacctaccagtgtctgaagggaacaggtgaaaactatcgcgggaatgtggctgttaccgtgtccgggcacacctgtcagcactggagtgcacagacccctcacacacataacaggacaccagaaaacttcccctgcaaaaatttggatgaaaactactgccgcaatcctgacggaaaaagggccccatggtgccatacaaccaacagccaagtgcggtgggagtactgtaagataccgtcctgtgactcctccccagtatccacggaacaattggctcccacagcaccacctgagctaacccctgtggtccaggactgctaccatggtgatggacagagctaccgaggcacatcctccaccaccaccacaggaaagaagtgtcagtcttggtcatctatgacaccacaccggcaccagaagaccccagaaaactacccaaatgctggcctgacaatgaactactgcaggaatccagatgccgataaaggcccctggtgttttaccacagaccccagcgtcaggtgggagtactgcaacctgaaaaaatgctcaggaacagaagcgagtgttgtagcacctccgcctgttgtcctgcttccagatgtagagactccttccgaagaagactgtatgtttgggaatgggaaaggataccgaggcaagagggcgaccactgttactgggacgccatgccaggactgggctgcccaggagccccatagacacagcattttcactccagagacaaatccacgggcgggtctggaaaaaaattactgccgtaaccctgatggtgatgtaggtggtccctggtgctacacgacaaatccaagatagctcgagtcaccagg

SEQ ID NO: 12 - DNA, 570 bp, Homo sapiens
atgctggggagcagagctgtaatgctgctgttgctgctgccctggacagctcagggcagagctgtgcctgggggcagcagccctgcctggactcagtgccagcagctttcacagaagctctgcacactggcctggagtgcacatccactagtgggacacatggatctaagagaagagggagatgaagagactacaaatgatgttccccatatccagtgtggagatggctgtgacccccaaggactcagggacaacagtcagttctgcttgcaaaggatccaccagggtctgattttttatgagaagctgctaggatcggatattttcacaggggagccttctctgctccctgatagccctgtgggccagcttcatgcctccctactgggcctcagccaactcctgcagcctgagggtcaccactgggagactcagcagattccaagcctcagtcccagccagccatggcagcgtctccttctccgcttcaaaatccttcgcagcctccaggcctttgtggctgtagccgcccgggtctttgcccatggagcagcaaccctgagtccctaa

SEQ ID NO: 13 - DNA, 2141 bp, Artificial Sequence
  human IL-23A - IRES - p40 cloned into modified E1b-19k region with flanking adenoviral sequences
ctgacctcgtcgacatgctggggagcagagctgtaatgctgctgttgctgctgccctggacagctcagggcagagctgtgcctgggggcagcagccctgcctggactcagtgccagcagctttcacagaagctctgcacactggcctggagtgcacatccactagtgggacacatggatctaagagaagagggagatgaagagactacaaatgatgttccccatatccagtgtggagatggctgtgacccccaaggactcagggacaacagtcagttctgcttgcaaaggatccaccagggtctgattttttatgagaagctgctaggatcggatattttcacaggggagccttctctgctccctgatagccctgtgggccagcttcatgcctccctactgggcctcagccaactcctgcagcctgagggtcaccactgggagactcagcagattccaagcctcagtcccagccagccatggcagcgtctccttctccgcttcaaaatccttcgcagcctccaggcctttgtggctgtagccgcccgggtctttgcccatggagcagcaaccctgagtccctaataacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttagtaactcgagtcaccagg

SEQ ID NO: 14 - DNA, 36808 bp, Artificial Sequence
  Adenovirus with human CD80 - IRES - CD137L cloned into modified E1b-19k region
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgttttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagctgacgtgtagtgtatttatacccggtgagttcctcaagaggccactcttgagtgccagcgagtagagttttctcctccgagccgctccgacaccgggactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtgattgcgtgtgtggttaacgcctttgtttgctgaatgagttgatgtaagtttaataaagggtgagataatgtttaacttgcatggcgtgttaaatggggcggggcttaaagggtatataatgcgccgtgggctaatcttggttacatctgacctcgtcgacatgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttgccccaagatgcagagagagaaggaggaatgagagattgagaagggaaagtgtacgccctgtataataacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggaatacgcctctgacgcttcactggaccccgaagccccgtggcctcctgcacctcgcgctcgcgcctgccgcgtactgccttgggccctggtcgcggggctgctgctcctgctcctgctcgctgctgcatgcgctgtatttcttgcatgcccatgggctgtgtctggggctcgcgcatcacctggctccgcggccagcccgagactccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaataactcgagtcaccaggcgcttttccaagagaaggtcatcaagactttggatttttccacaccggggcgcgctgcggctgctgttgcttttttgagttttataaaggataaatggagcgaagaaacccatctgagcggggggtaccctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgtccttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccttggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagggttggggttattctctgtcttgtgattctctttattcttatactaacgcttctctgcctaaggctcgccgcctgctgtgtgcacatttgcatttattgtcagctttttaaacgctggggtcgccacccaagatgattaggtacataatcctaggtttactcacccttgcgtcagcccacggtaccacccaaaaggtggattttaaggagccagcctgtaatgttacattcgcagctgaagctaatgagtgcaccactcttataaaatgcaccacagaacatgaaaagctgcttattcgccacaaaaacaaaattggcaagtatgctgtttatgctatttggcagccaggtgacactacagagtataatgttacagttttccagggtaaaagtcataaaacttttatgtatacttttccattttatgaaatgtgcgacattaccatgtacatgagcaaacagtataagttgtggcccccacaaaattgtgtggaaaacactggcactttctgctgcactgctatgctaattacagtgctcgctttggtctgtaccctactctatattaaatacaaaagcagacgcagctttattgaggaaaagaaaatgccttaatttactaagttacaaagctaatgtcaccactaactgctttactcgctgcttgcaaaacaaattcaaaaagttagcattataattagaataggatttaaaccccccggtcatttcctgctcaataccattcccctgaacaattgactctatgtgggatatgctccagcgctacaaccttgaagtcaggcttcctggatgtcagcatctgactttggccagcacctgtcccgcggatttgttccagtccaactacagcgacccaccctaacagagatgaccaacacaaccaacgcggccgccgctaccggacttacatctaccacaaatacaccccaagtttctgcctttgtcaataactgggataacttgggcatgtggtggttctccatagcgcttatgtttgtatgccttattattatgtggctcatctgctgcctaaagcgcaaacgcgcccgaccacccatctatagtcccatcattgtgctacacccaaacaatgatggaatccatagattggacggactgaaacacatgttcttttctcttacagtatgataataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccttgacaaaagaacccacactgattatgacacgcatactcggagctatgctaaccagcgtagccccgatgtaagctttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttcacagcggcagcctaacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtaacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatg

SEQ ID NO: 15 - DNA, 46 bp, Artificial Sequence
  Modified E1b-19k reigon
atcttggttacatctgacctcgtcgagtcaccaggcgcttttccaa

SEQ ID NO: 16 - DNA, 51 bp, Artificial Sequence
  Modified E3 region
tcttttctcttacagtatgataataaaaaaaaataataaagcatcacttac

SEQ ID NO: 17 - DNA, 953 bp, Artificial Sequence
  murine CD80 cloned into E1b-19k region with flanking adenoviral sequences
atctgacctcgtcgacatggcttgcaattgtcagttgatgcaggatacaccactcctcaagtttccatgtccaaggctcattcttctctttgtgctgctgattcgtctttcacaagtgtcttcagatgttgatgaacaactgtccaagtcagtgaaagataaggtattgctgccttgccgttacaactctcctcatgaagatgagtctgaagaccgaatctactggcaaaaacatgacaaagtggtgctgtctgtcattgctgggaaactaaaagtgtggcccgagtataagaaccggactttatatgacaacactacctactctcttatcatcctgggcctggtcctttcagaccggggcacatacagctgtgtcgttcaaaagaaggaaagaggaacgtatgaagttaaacacttggctttagtaaagttgtccatcaaagctgacttctctacccccaacataactgagtctggaaacccatctgcagacactaaaaggattacctgctttgcttccgggggtttcccaaagcctcgcttctcttggttggaaaatggaagagaattacctggcatcaatacgacaatttcccaggatcctgaatctgaattgtacaccattagtagccaactagatttcaatacgactcgcaaccacaccattaagtgtctcattaaatatggagatgctcacgtgtcagaggacttcacctgggaaaaacccccagaagaccctcctgatagcaagaacacacttgtgctctttggggcaggattcggcgcagtaataacagtcgtcgtcatcgttgtcatcatcaaatgcttctgtaagcacagaagctgtttcagaagaaatgaggcaagcagagaaacaaacaacagccttaccttcgggcctgaagaagcattagctgaacagaccgtcttcctttagctcgagtcaccaggcg

SEQ ID NO: 18 - DNA, 947 bp, Artificial Sequence
  human CD80 cloned into E1b-19k region with flanking adenoviral sequences
gcgccgtgggctaatcttggttacatctgacctcgtcgacatgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttgccccaagatgcagagagagaaggaggaatgagagattgagaagggaaagtgtacgccctgtataactcgagtcaccaggcgcttttccaagagaaggtcatcaag

SEQ ID NO: 19 - DNA, 999 bp, Artificial Sequence
  murine CD137L cloned into modified E3 region with flanking adenoviral sequences
atgttcttttctcttacagtatgattaaatgagacatggaccagcacacacttgatgtggaggataccgcggatgccagacatccagcaggtacttcgtgcccctcggatgcggcgctcctcagagataccgggctcctcgcggacgctgcgctcctctcagatactgtgcgccccacaaatgccgcgctccccacggatgctgcctaccctgcggttaatgttcgggatcgcgaggccgcgtggccgcctgcactgaacttctgttcccgccacccaaagctctatggcctagtcgctttggttttgctgcttctgatcgccgcctgtgttcctatcttcacccgcaccgagcctcggccagcgctcacaatcaccacctcgcccaacctgggtacccgagagaataatgcagaccaggtcacccctgtttcccacattggctgccccaacactacacaacagggctctcctgtgttcgccaagctactggctaaaaaccaagcatcgttgtgcaatacaactctgaactggcacagccaagatggagctgggagctcatacctatctcaaggtctgaggtacgaagaagacaaaaaggagttggtggtagacagtcccgggctctactacgtatttttggaactgaagctcagtccaacattcacaaacacaggccacaaggtgcagggctgggtctctcttgttttgcaagcaaagcctcaggtagatgactttgacaacttggccctgacagtggaactgttcccttgctccatggagaacaagttagtggaccgttcctggagtcaactgttgctcctgaaggctggccaccgcctcagtgtgggtctgagggcttatctgcatggagcccaggatgcatacagagactgggagctgtcttatcccaacaccaccagctttggactctttcttgtgaaacccgacaacccatgggaatgaggtctcaaagatcttattccctttaactaataaa

SEQ ID NO: 20 - DNA, 834 bp, Artificial Sequence
  human CD137L cloned into modified E3 region with flanking adenoviral sequences
atgttcttttctcttacagtatgattaaatgagacatggaatacgcctctgacgcttcactggaccccgaagccccgtggcctcctgcacctcgcgctcgcgcctgccgcgtactgccttgggccctggtcgcggggctgctgctcctgctcctgctcgctgctgcatgcgctgtatttcttgcatgcccatgggctgtgtctggggctcgcgcatcacctggctccgcggccagcccgagactccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaataaggtctcaaagatcttattccctttaactaataaa

SEQ ID NO: 21 - DNA, 2212 bp, Artificial Sequence
  human CD80 - IRES - CD137L cloned into modified E1b-19k region with flanking adenoviral sequences
ctgacctcgtcgacatgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttgccccaagatgcagagagagaaggaggaatgagagattgagaagggaaagtgtacgccctgtataataacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggaatacgcctctgacgcttcactggaccccgaagccccgtggcctcctgcacctcgcgctcgcgcctgccgcgtactgccttgggccctggtcgcggggctgctgctcctgctcctgctcgctgctgcatgcgctgtatttcttgcatgcccatgggctgtgtctggggctcgcgcatcacctggctccgcggccagcccgagactccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaataactcgagtcaccag

SEQ ID NO: 22 - DNA, 36845 bp, Artificial Sequence
  Adenovirus with human CD80 - IRES - CD137L - IRES - ICAM1 cloned into modified E1b-19k region
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgttttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagctgacgtgtagtgtatttatacccggtgagttcctcaagaggccactcttgagtgccagcgagtagagttttctcctccgagccgctccgacaccgggactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtgattgcgtgtgtggttaacgcctttgtttgctgaatgagttgatgtaagtttaataaagggtgagataatgtttaacttgcatggcgtgttaaatggggcggggcttaaagggtatataatgcgccgtgggctaatcttggttacatctgacctcgtcgacatgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttgccccaagatgcagagagagaaggaggaatgagagattgagaagggaaagtgtacgccctgtataacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggaatacgcctctgacgcttcactggaccccgaagccccgtggcctcccgcgccccgcgctcgcgcctgccgcgtactgccttgggccctggtcgcggggctgctgctgctgctgctgctcgctgccgcctgcgccgtcttcctcgcctgcccctgggccgtgtccggggctcgcgcctcgcccggctccgcggccagcccgagactccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaaggtttccacaactgataaaactcgtgcaacttgaaactccgcctggtctttccaggtctagaggggttacactttgtactgtgctcgactccacgcccggtccactggcgggtgttagtagcagcactgttgtttcgtagcggagcatggtggccgtgggaactcctccttggtgacaagggcccacggggccgaaagccacgtccagacggacccaccatgtgtgcaaccccagcacggcaacttttactgcgaacaccaccttaaggtgacactggtactggtactcggtcactggtgacaggctaaggatgcccttcaggtaccccgaggtaacacgggacactcgggatctgagaaggggattgggacttctttaaaagtgcccagtttaaaaagcttctacgcctgaataggcgaccggaggccggcgcctttccattacccactactaaatccatggctcccagcagcccccggcccgcgctgcccgcactcctggtcctgctcggggctctgttcccaggacctggcaatgcccagacatctgtgtccccctcaaaagtcatcctgccccggggaggctccgtgctggtgacatgcagcacctcctgtgaccagcccaagttgttgggcatagagaccccgttgcctaaaaaggagttgctcctgcctgggaacaaccggaaggtgtatgaactgagcaatgtgcaagaagatagccaaccaatgtgctattcaaactgccctgatgggcagtcaacagctaaaaccttcctcaccgtgtactggactccagaacgggtggaactggcacccctcccctcttggcagccagtgggcaagaaccttaccctacgctgccaggtggagggtggggcaccccgggccaacctcaccgtggtgctgctccgtggggagaaggagctgaaacgggagccagctgtgggggagcccgctgaggtcacgaccacggtgctggtgaggagagatcaccatggagccaatttctcgtgccgcactgaactggacctgcggccccaagggctggagctgtttgagaacacctcggccccctaccagctccagacctttgtcctgccagcgactcccccacaacttgtcagcccccgggtcctagaggtggacacgcaggggaccgtggtctgttccctggacgggctgttcccagtctcggaggcccaggtccacctggcactgggggaccagaggttgaaccccacagtcacctatggcaacgactccttctcggccaaggcctcagtcagtgtgaccgcagaggacgagggcacccagcggctgacgtgtgcagtaatactggggaaccagagccaggagacactgcagacagtgaccatctacagctttccggcgcccaacgtgattctgacgaagccagaggtctcagaagggaccgaggtgacagtgaagtgtgaggcccaccctagagccaaggtgacgctgaatggggttccagcccagccactgggcccgagggcccagctcctgctgaaggccaccccagaggacaacgggcgcagcttctcctgctctgcaaccctggaggtggccggccagcttatacacaagaaccagacccgggagcttcgtgtcctgtatggcccccgactggacgagagggattgtccgggaaactggacgtggccagaaaattcccagcagactccaatgtgccaggcttgggggaacccattgcccgagctcaagtgtctaaaggatggcactttcccactgcccatcggggaatcagtgactgtcactcgagatcttgagggcacctacctctgtcgggccaggagcactcaaggggaggtcacccgcaaggtgaccgtgaatgtgctctccccccggtatgagattgtcatcatcactgtggtagcagccgcagtcataatgggcactgcaggcctcagcacgtacctctataaccgccagcggaagatcaagaaatacagactacaacaggcccaaaaagggacccccatgaaaccgaacacacaagccacgcctccctgactcgagtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgtccttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccttggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagggttggggttattctctgtcttgtgattctctttattcttatactaacgcttctctgcctaaggctcgccgcctgctgtgtgcacatttgcatttattgtcagctttttaaacgctggggtcgccacccaagatgattaggtacataatcctaggtttactcacccttgcgtcagcccacggtaccacccaaaaggtggattttaaggagccagcctgtaatgttacattcgcagctgaagctaatgagtgcaccactcttataaaatgcaccacagaacatgaaaagctgcttattcgccacaaaaacaaaattggcaagtatgctgtttatgctatttggcagccaggtgacactacagagtataatgttacagttttccagggtaaaagtcataaaacttttatgtatacttttccattttatgaaatgtgcgacattaccatgtacatgagcaaacagtataagttgtggcccccacaaaattgtgtggaaaacactggcactttctgctgcactgctatgctaattacagtgctcgctttggtctgtaccctactctatattaaatacaaaagcagacgcagctttattgaggaaaagaaaatgccttaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtaacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatg

SEQ ID NO: 23 - DNA, 35938 bp, Adenovrius type 5
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagctgacgtgtagtgtatttatacccggtgagttcctcaagaggccactcttgagtgccagcgagtagagttttctcctccgagccgctccgacaccgggactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtgattgcgtgtgtggttaacgcctttgtttgctgaatgagttgatgtaagtttaataaagggtgagataatgtttaacttgcatggcgtgttaaatggggcggggcttaaagggtatataatgcgccgtgggctaatcttggttacatctgacctcatggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgtccttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccttggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaatcggggttggggttattctctgtcttgtgattctctttattcttatactaacgcttctctgcctaaggctcgccgcctgctgtgtgcacatttgcatttattgtcagctttttaaacgctggggtcgccacccaagatgattaggtacataatcctaggtttactcacccttgcgtcagcccacggtaccacccaaaaggtggattttaaggagccagcctgtaatgttacattcgcagctgaagctaatgagtgcaccactcttataaaatgcaccacagaacatgaaaagctgcttattcgccacaaaaacaaaattggcaagtatgctgtttatgctatttggcagccaggtgacactacagagtataatgttacagttttccagggtaaaagtcataaaacttttatgtatacttttccattttatgaaatgtgcgacattaccatgtacatgagcaaacagtataagttgtggcccccacaaaattgtgtggaaaacactggcactttctgctgcactgctatgctaattacagtgctcgctttggtctgtaccctactctatattaaatacaaaagcagacgcagctttattgaggaaaagaaaatgccttaatttactaagttacaaagctaatgtcaccactaactgctttactcgctgcttgcaaaacaaattcaaaaagttagcattataattagaataggatttaaaccccccggtcatttcctgctcaataccattcccctgaacaattgactctatgtgggatatgctccagcgctacaaccttgaagtcaggcttcctggatgtcagcatctgactttggccagcacctgtcccgcggatttgttccagtccaactacagcgacccaccctaacagagatgaccaacacaaccaacgcggccgccgctaccggacttacatctaccacaaatacaccccaagtttctgcctttgtcaataactgggataacttgggcatgtggtggttctccatagcgcttatgtttgtatgccttattattatgtggctcatctgctgcctaaagcgcaaacgcgcccgaccacccatctatagtcccatcattgtgctacacccaaacaatgatggaatccatagattggacggactgaaacacatgttcttttctcttacagtatgattaaatgagacatgattcctcgagtttttatattactgacccttgttgcgcttttttgtgcgtgctccacattggctgcggtttctcacatcgaagtagactgcattccagccttcacagtctatttgctttacggatttgtcaccctcacgctcatctgcagcctcatcactgtggtcatcgcctttatccagtgcattgactgggtctgtgtgcgctttgcatatctcagacaccatccccagtacagggacaggactatagctgagcttcttagaattctttaattatgaaatttactgtgacttttctgctgattatttgcaccctatctgcgttttgttccccgacctccaagcctcaaagacatatatcatgcagattcactcgtatatggaatattccaagttgctacaatgaaaaaagcgatctttccgaagcctggttatatgcaatcatctctgttatggtgttctgcagtaccatcttagccctagctatatatccctaccttgacattggctggaaacgaatagatgccatgaaccacccaactttccccgcgcccgctatgcttccactgcaacaagttgttgccggcggctttgtcccagccaatcagcctcgccccacttctcccacccccactgaaatcagctactttaatctaacaggaggagatgactgacaccctagatctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtaacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatg

SEQ ID NO: 24 - DNA, 54 bp, Artificial Sequence
  Modified E3 region
ttattgaggaaaagaaaatgccttaataaaaaaaaataataaagcatcacttac

SEQ ID NO: 25 - DNA, 41 bp, Artificial Sequence
  Modified E4 region
gaacgccggacgtagtcataacagtcagccttaccagtaaa

SEQ ID NO: 26 - DNA, 4509 bp, Artificial Sequence
  murine CD80 - EMCV IRES - CD137L - FMDV IRES - ICAM-1 cloned into modified E1b-19k region with flanking adenoviral sequences
atctgacctcgtcgacatggcttgcaattgtcagttgatgcaggatacaccactcctcaagtttccatgtccaaggctcattcttctctttgtgctgctgattcgtctttcacaagtgtcttcagatgttgatgaacaactgtccaagtcagtgaaagataaggtattgctgccttgccgttacaactctcctcatgaagatgagtctgaagaccgaatctactggcaaaaacatgacaaagtggtgctgtctgtcattgctgggaaactaaaagtgtggcccgagtataagaaccggactttatatgacaacactacctactctcttatcatcctgggcctggtcctttcagaccggggcacatacagctgtgtcgttcaaaagaaggaaagaggaacgtatgaagttaaacacttggctttagtaaagttgtccatcaaagctgacttctctacccccaacataactgagtctggaaacccatctgcagacactaaaaggattacctgctttgcttccgggggtttcccaaagcctcgcttctcttggttggaaaatggaagagaattacctggcatcaatacgacaatttcccaggatcctgaatctgaattgtacaccattagtagccaactagatttcaatacgactcgcaaccacaccattaagtgtctcattaaatatggagatgctcacgtgtcagaggacttcacctgggaaaaacccccagaagaccctcctgatagcaagaacacacttgtgctctttggggcaggattcggcgcagtaataacagtcgtcgtcatcgttgtcatcatcaaatgcttctgtaagcacagaagctgtttcagaagaaatgaggcaagcagagaaacaaacaacagccttaccttcgggcctgaagaagcattagctgaacagaccgtcttcctttagtaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggaccagcacacacttgatgtggaggataccgcggatgccagacatccagcaggtacttcgtgcccctcggatgcggcgctcctcagagataccgggctcctcgcggacgctgcgctcctctcagatactgtgcgccccacaaatgccgcgctccccacggatgctgcctaccctgcggttaatgttcgggatcgcgaggccgcgtggccgcctgcactgaacttctgttcccgccacccaaagctctatggcctagtcgctttggttttgctgcttctgatcgccgcctgtgttcctatcttcacccgcaccgagcctcggccagcgctcacaatcaccacctcgcccaacctgggtacccgagagaataatgcagaccaggtcacccctgtttcccacattggctgccccaacactacacaacagggctctcctgtgttcgccaagctactggctaaaaaccaagcatcgttgtgcaatacaactctgaactggcacagccaagatggagctgggagctcatacctatctcaaggtctgaggtacgaagaagacaaaaaggagttggtggtagacagtcccgggctctactacgtatttttggaactgaagctcagtccaacattcacaaacacaggccacaaggtgcagggctgggtctctcttgttttgcaagcaaagcctcaggtagatgactttgacaacttggccctgacagtggaactgttcccttgctccatggagaacaagttagtggaccgttcctggagtcaactgttgctcctgaaggctggccaccgcctcagtgtgggtctgagggcttatctgcatggagcccaggatgcatacagagactgggagctgtcttatcccaacaccaccagctttggactctttcttgtgaaacccgacaacccatgggaatgaggtttccacaactgataaaactcgtgcaacttgaaactccgcctggtctttccaggtctagaggggttacactttgtactgtgctcgactccacgcccggtccactggcgggtgttagtagcagcactgttgtttcgtagcggagcatggtggccgtgggaactcctccttggtgacaagggcccacggggccgaaagccacgtccagacggacccaccatgtgtgcaaccccagcacggcaacttttactgcgaacaccaccttaaggtgacactggtactggtactcggtcactggtgacaggctaaggatgcccttcaggtaccccgaggtaacacgggacactcgggatctgagaaggggattgggacttctttaaaagtgcccagtttaaaaagcttctacgcctgaataggcgaccggaggccggcgcctttccattacccactactaaatccatggcttcaacccgtgccaagcccacgctacctctgctcctggccctggtcaccgttgtgatccctgggcctggtgatgctcaggtatccatccatcccagagaagccttcctgccccagggtgggtccgtgcaggtgaactgttcttcctcatgcaaggaggacctcagcctgggcttggagactcagtggctgaaagatgagctcgagagtggacccaactggaagctgtttgagctgagcgagatcggggaggacagcagtccgctgtgctttgagaactgtggcaccgtgcagtcgtccgcttccgctaccatcaccgtgtattcgtttccggagagtgtggagctgagacctctgccagcctggcagcaagtaggcaaggacctcaccctgcgctgccacgtggatggtggagcaccgcggacccagctctcagcagtgctgctccgtggggaggagatactgagccgccagccagtgggtgggcaccccaaggaccccaaggagatcacattcacggtgctggctagcagaggggaccacggagccaatttctcatgccgcacagaactggatctcaggccgcaagggctggcattgttctctaatgtctccgaggccaggagcctccggactttcgatcttccagctaccatcccaaagctcgacacccctgacctcctggaggtgggcacccagcagaagttgttttgctccctggaaggcctgtttcctgcctctgaagctcggatatacctggagctgggaggccagatgccgacccaggagagcacaaacagcagtgactctgtgtcagccactgccttggtagaggtgactgaggagttcgacagaaccctgccgctgcgctgcgttttggagctagcggaccagatcctggagacgcagaggaccttaacagtctacaacttttcagctccggtcctgaccctgagccagctggaggtctcggaagggagccaagtaactgtgaagtgtgaagcccacagtgggtcgaaggtggttcttctgagcggcgtcgagcctaggccacccaccccgcaggtccaattcacactgaatgccagctcggaggatcacaaacgaagcttcttttgctctgccgctctggaggtggcgggaaagttcctgtttaaaaaccagaccctggaactgcacgtgctgtatggtcctcggctggacgagacggactgcttggggaactggacctggcaagaggggtctcagcagactctgaaatgccaggcctgggggaacccatctcctaagatgacctgcagacggaaggcagatggtgccctgctgcccatcggggtggtgaagtctgtcaaacaggagatgaatggtacatacgtgtgccatgcctttagctcccatgggaatgtcaccaggaatgtgtacctgacagtactgtaccactctcaaaataactggactataatcattctggtgccagtactgctggtcattgtgggcctcgtgatggcagcctcttatgtttataaccgccagagaaagatcaggatatacaagttacagaaggctcaggaggaggccataaaactcaagggacaagccccacctccctgactcgagtcaccaggcg

SEQ ID NO: 27 - DNA, 2220 bp, Artificial Sequence
  human CD80 - IRES - CD137L cloned into modified E1b-19k region with flanking adenoviral sequences
gcgccgtgggctaatcttggttacatctgacctcgtcgacatgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttgccccaagatgcagagagagaaggaggaatgagagattgagaagggaaagtgtacgccctgtataataacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggaatacgcctctgacgcttcactggaccccgaagccccgtggcctcctgcacctcgcgctcgcgcctgccgcgtactgccttgggccctggtcgcggggctgctgctcctgctcctgctcgctgctgcatgcgctgtatttcttgcatgcccatgggctgtgtctggggctcgcgcatcacctggctccgcggccagcccgagactccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcgg

SEQ ID NO: 28 - DNA, 10 bp, Artificial Sequence
  Sequence resulting from TAV-255 deletion
ggtgttttgg

SEQ ID NO: 29 - DNA, 8 bp, Adenovirus type 5
tgccttaa

SEQ ID NO: 30 - DNA, 11 bp, Adenovirus type 5
taaaaaaaaat

SEQ ID NO: 31 - DNA, 4275 bp, Artificial Sequence
  human CD80 - EMCV IRES - CD137L - FMDV IRES - ICAM-1 cloned into modified E1b-19k region with flanking adenoviral sequences
atctgacctcgtcgacatgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttgccccaagatgcagagagagaaggaggaatgagagattgagaagggaaagtgtacgccctgtataataacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggaatacgcctctgacgcttcactggaccccgaagccccgtggcctcctgcacctcgcgctcgcgcctgccgcgtactgccttgggccctggtcgcggggctgctgctcctgctcctgctcgctgctgcatgcgctgtatttcttgcatgcccatgggctgtgtctggggctcgcgcatcacctggctccgcggccagcccgagactccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaataaggtttccacaactgataaaactcgtgcaacttgaaactccgcctggtctttccaggtctagaggggttacactttgtactgtgctcgactccacgcccggtccactggcgggtgttagtagcagcactgttgtttcgtagcggagcatggtggccgtgggaactcctccttggtgacaagggcccacggggccgaaagccacgtccagacggacccaccatgtgtgcaaccccagcacggcaacttttactgcgaacaccaccttaaggtgacactggtactggtactcggtcactggtgacaggctaaggatgcccttcaggtaccccgaggtaacacgggacactcgggatctgagaaggggattgggacttctttaaaagtgcccagtttaaaaagcttctacgcctgaataggcgaccggaggccggcgcctttccattacccactactaaatccatggctcccagcagcccccggcccgcgctgcccgcactcctggtcctgctcggggctctgttcccaggacctggcaatgcccagacatctgtgtccccctcaaaagtcatcctgccccggggaggctccgtgctggtgacatgcagcacctcctgtgaccagcccaagttgttgggcatagagaccccgttgcctaaaaaggagttgctcctgcctgggaacaaccggaaggtgtatgaactgagcaatgtgcaagaagatagccaaccaatgtgctattcaaactgccctgatgggcagtcaacagctaaaaccttcctcaccgtgtactggactccagaacgggtggaactggcacccctcccctcttggcagccagtgggcaagaaccttaccctacgctgccaggtggagggtggggcaccccgggccaacctcaccgtggtgctgctccgtggggagaaggagctgaaacgggagccagctgtgggggagcccgctgaggtcacgaccacggtgctggtgaggagagatcaccatggagccaatttctcgtgccgcactgaactggacctgcggccccaagggctggagctgtttgagaacacctcggccccctaccagctccagacctttgtcctgccagcgactcccccacaacttgtcagcccccgggtcctagaggtggacacgcaggggaccgtggtctgttccctggacgggctgttcccagtctcggaggcccaggtccacctggcactgggggaccagaggttgaaccccacagtcacctatggcaacgactccttctcggccaaggcctcagtcagtgtgaccgcagaggacgagggcacccagcggctgacgtgtgcagtaatactggggaaccagagccaggagacactgcagacagtgaccatctacagctttccggcgcccaacgtgattctgacgaagccagaggtctcagaagggaccgaggtgacagtgaagtgtgaggcccaccctagagccaaggtgacgctgaatggggttccagcccagccactgggcccgagggcccagctcctgctgaaggccaccccagaggacaacgggcgcagcttctcctgctctgcaaccctggaggtggccggccagcttatacacaagaaccagacccgggagcttcgtgtcctgtatggcccccgactggacgagagggattgtccgggaaactggacgtggccagaaaattcccagcagactccaatgtgccaggcttgggggaacccattgcccgagctcaagtgtctaaaggatggcactttcccactgcccatcggggaatcagtgactgtcactcgagatcttgagggcacctacctctgtcgggccaggagcactcaaggggaggtcacccgcaaggtgaccgtgaatgtgctctccccccggtatgagattgtcatcatcactgtggtagcagccgcagtcataatgggcactgcaggcctcagcacgtacctctataaccgccagcggaagatcaagaaatacagactacaacaggcccaaaaagggacccccatgaaaccgaacacacaagccacgcctccctgactcgagtcaccaggcg

SEQ ID NO: 32 - DNA, 1599 bp, Homo sapiens
atggctcccagcagcccccggcccgcgctgcccgcactcctggtcctgctcggggctctgttcccaggacctggcaatgcccagacatctgtgtccccctcaaaagtcatcctgccccggggaggctccgtgctggtgacatgcagcacctcctgtgaccagcccaagttgttgggcatagagaccccgttgcctaaaaaggagttgctcctgcctgggaacaaccggaaggtgtatgaactgagcaatgtgcaagaagatagccaaccaatgtgctattcaaactgccctgatgggcagtcaacagctaaaaccttcctcaccgtgtactggactccagaacgggtggaactggcacccctcccctcttggcagccagtgggcaagaaccttaccctacgctgccaggtggagggtggggcaccccgggccaacctcaccgtggtgctgctccgtggggagaaggagctgaaacgggagccagctgtgggggagcccgctgaggtcacgaccacggtgctggtgaggagagatcaccatggagccaatttctcgtgccgcactgaactggacctgcggccccaagggctggagctgtttgagaacacctcggccccctaccagctccagacctttgtcctgccagcgactcccccacaacttgtcagcccccgggtcctagaggtggacacgcaggggaccgtggtctgttccctggacgggctgttcccagtctcggaggcccaggtccacctggcactgggggaccagaggttgaaccccacagtcacctatggcaacgactccttctcggccaaggcctcagtcagtgtgaccgcagaggacgagggcacccagcggctgacgtgtgcagtaatactggggaaccagagccaggagacactgcagacagtgaccatctacagctttccggcgcccaacgtgattctgacgaagccagaggtctcagaagggaccgaggtgacagtgaagtgtgaggcccaccctagagccaaggtgacgctgaatggggttccagcccagccactgggcccgagggcccagctcctgctgaaggccaccccagaggacaacgggcgcagcttctcctgctctgcaaccctggaggtggccggccagcttatacacaagaaccagacccgggagcttcgtgtcctgtatggcccccgactggacgagagggattgtccgggaaactggacgtggccagaaaattcccagcagactccaatgtgccaggcttgggggaacccattgcccgagctcaagtgtctaaaggatggcactttcccactgcccatcggggaatcagtgactgtcactcgagatcttgagggcacctacctctgtcgggccaggagcactcaaggggaggtcacccgcaaggtgaccgtgaatgtgctctccccccggtatgagattgtcatcatcactgtggtagcagccgcagtcataatgggcactgcaggcctcagcacgtacctctataaccgccagcggaagatcaagaaatacagactacaacaggcccaaaaagggacccccatgaaaccgaacacacaagccacgcctccctga

SEQ ID NO: 33 - protein, 1516 aa, Homo sapiens
MAPYPCGCHILLLLFCCLAAARANLLNLNWLWFNNEDTSHAATTIPEPQGPLPVQPTADTTTHVTPRNGSTEPATAPGSPEPPSELLEDGQDTPTSAESPDAPEENIAGVGAEILNVAKGIRSFVQLWNDTVPTESLARAETLVLETPVGPLALAGPSSTPQENGTTLWPSRGIPSSPGAHTTEAGTLPAPTPSPPSLGRPWAPLTGPSVPPPSSERISEEVGLLQLLGDPPPQQVTQTDDPDVGLAYVFGPDANSGQVARYHFPSLFFRDFSLLFHIRPATEGPGVLFAITDSAQAMVLLGVKLSGVQDGHQDISLLYTEPGAGQTHTAASFRLPAFVGQWTHLALSVAGGFVALYVDCEEFQRMPLARSSRGLELEPGAGLFVAQAGGADPDKFQGVIAELKVRRDPQVSPMHCLDEEGDDSDGASGDSGSGLGDARELLREETGAALKPRLPAPPPVTTPPLAGGSSTEDSRSEEVEEQTTVASLGAQTLPGSDSVSTWDGSVRTPGGRVKEGGLKGQKGEPGVPGPPGRAGPPGSPCLPGPPGLPCPVSPLGPAGPALQTVPGPQGPPGPPGRDGTPGRDGEPGDPGEDGKPGDTGPQGFPGTPGDVGPKGDKGDPGVGERGPPGPQGPPGPPGPSFRHDKLTFIDMEGSGFGGDLEALRGPRGFPGPPGPPGVPGLPGEPGRFGVNSSDVPGPAGLPGVPGREGPPGFPGLPGPPGPPGREGPPGRTGQKGSLGEAGAPGHKGSKGAPGPAGARGESGLAGAPGPAGPPGPPGPPGPPGPGLPAGFDDMEGSGGPFWSTARSADGPQGPPGLPGLKGDPGVPGLPGAKGEVGADGVPGFPGLPGREGIAGPQGPKGDRGSRGEKGDPGKDGVGQPGLPGPPGPPGPVVYVSEQDGSVLSVPGPEGRPGFAGFPGPAGPKGNLGSKGERGSPGPKGEKGEPGSIFSPDGGALGPAQKGAKGEPGFRGPPGPYGRPGYKGEIGFPGRPGRPGMNGLKGEKGEPGDASLGFGMRGMPGPPGPPGPPGPPGTPVYDSNVFAESSRPGPPGLPGNQGPPGPKGAKGEVGPPGPPGQFPFDFLQLEAEMKGEKGDRGDAGQKGERGEPGGGGFFGSSLPGPPGPPGPRGYPGIPGPKGESIRGQPGPPGPQGPPGIGYEGRQGPPGPPGPPGPPSFPGPHRQTISVPGPPGPPGPPGPPGTMGASSGVRLWATRQAMLGQVHEVPEGWLIFVAEQEELYVRVQNGFRKVQLEARTPLPRGTDNEVAALQPPVVQLHDSNPYPRREHPHPTARPWRADDILASPPRLPEPQPYPGAPHHSSYVHLRPARPTSPPAHSHRDFQPVLHLVALNSPLSGGMRGIRGADFQCFQQARAVGLAGTFRAFLSSRLQDLYSIVRRADRAAVPIVNLKDELLFPSWEALFSGSEGPLKPGARIFSFDGKDVLRHPTWPQKSVWHGSDPNGRRLTESYCETWRTEAPSATGQASSLLGGRLLGQSAASCHHAYIVLCIENSFMTASK

SEQ ID NO: 34 - protein, 810 aa, Homo sapiens
MEHKEVVLLLLLFLKSGQGEPLDDYVNTQGASLFSVTKKQLGAGSIEECAAKCEEDEEFTCRAFQYHSKEQQCVIMAENRKSSIIIRMRDVVLFEKKVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 35 - DNA, 2673 bp, Artificial Sequence
  human endostatin - IRES - angiostatin cloned into modified E1b-19k region with flanking adenoviral sequences
atctgacctcgtcgacatggctccctacccctgtggctgccacatcctgctgctgctcttctgctgcctggcggctgcccgggccagctcctacgtgcacctgcggccggcgcgacccacaagcccacccgcccacagccaccgcgacttccagccggtgctccacctggttgcgctcaacagccccctgtcaggcggcatgcggggcatccgcggggccgacttccagtgcttccagcaggcgcgggccgtggggctggcgggcaccttccgcgccttcctgtcctcgcgcctgcaggacctgtacagcatcgtgcgccgtgccgaccgcgcagccgtgcccatcgtcaacctcaaggacgagctgctgtttcccagctgggaggctctgttctcaggctctgagggtccgctgaagcccggggcacgcatcttctcctttgacggcaaggacgtcctgaggcaccccacctggccccagaagagcgtgtggcatggctcggaccccaacgggcgcaggctgaccgagagctactgtgagacgtggcggacggaggctccctcggccacgggccaggcctcctcgctgctggggggcaggctcctggggcagagtgccgcgagctgccatcacgcctacatcgtgctctgcattgagaacagcttcatgactgcctccaagtagtaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggaacataaggaagtggttcttctacttcttttatttctgaaatcaggtcaaggaaaagtgtatctctcagagtgcaagactgggaatggaaagaactacagagggacgatgtccaaaacaaaaaatggcatcacctgtcaaaaatggagttccacttctccccacagacctagattctcacctgctacacacccctcagagggactggaggagaactactgcaggaatccagacaacgatccgcaggggccctggtgctatactactgatccagaaaagagatatgactactgcgacattcttgagtgtgaagaggaatgtatgcattgcagtggagaaaactatgacggcaaaatttccaagaccatgtctggactggaatgccaggcctgggactctcagagcccacacgctcatggatacattccttccaaatttccaaacaagaacctgaagaagaattactgtcgtaaccccgatagggagctgcggccttggtgtttcaccaccgaccccaacaagcgctgggaactttgtgacatcccccgctgcacaacacctccaccatcttctggtcccacctaccagtgtctgaagggaacaggtgaaaactatcgcgggaatgtggctgttaccgtgtccgggcacacctgtcagcactggagtgcacagacccctcacacacataacaggacaccagaaaacttcccctgcaaaaatttggatgaaaactactgccgcaatcctgacggaaaaagggccccatggtgccatacaaccaacagccaagtgcggtgggagtactgtaagataccgtcctgtgactcctccccagtatccacggaacaattggctcccacagcaccacctgagctaacccctgtggtccaggactgctaccatggtgatggacagagctaccgaggcacatcctccaccaccaccacaggaaagaagtgtcagtcttggtcatctatgacaccacaccggcaccagaagaccccagaaaactacccaaatgctggcctgacaatgaactactgcaggaatccagatgccgataaaggcccctggtgttttaccacagaccccagcgtcaggtgggagtactgcaacctgaaaaaatgctcaggaacagaagcgagtgttgtagcacctccgcctgttgtcctgcttccagatgtagagactccttccgaagaagactgtatgtttgggaatgggaaaggataccgaggcaagagggcgaccactgttactgggacgccatgccaggactgggctgcccaggagccccatagacacagcattttcactccagagacaaatccacgggcgggtctggaaaaaaattactgccgtaaccctgatggtgatgtaggtggtccctggtgctacacgacaaatccaagatagctcgagtcaccaggcg

SEQ ID NO: 36 - DNA, 2685 bp, Artificial Sequence
  murine endostatin - IRES - angiostatin cloned into modified E1b-19k region with flanking adenoviral sequences
atctgacctcgtcgacatggctcccgaccccagcagacgcctctgcctgctgctgctgttgctgctctcctgccgccttgtgcctgccagcgcttatgtgcacctgccgccagcccgccccaccctctcacttgctcatactcatcaggactttcagccagtgctccacctggtggcactgaacacccccctgtctggaggcatgcgtggtatccgtggagcagatttccagtgcttccagcaagcccgagccgtggggctgtcgggcaccttccgggctttcctgtcctctaggctgcaggatctctatagcatcgtgcgccgtgctgaccgggggtctgtgcccatcgtcaacctgaaggacgaggtgctatctcccagctgggactccctgttttctggctcccagggtcaactgcaacccggggcccgcatcttttcttttgacggcagagatgtcctgagacacccagcctggccgcagaagagcgtatggcacggctcggaccccagtgggcggaggctgatggagagttactgtgagacatggcgaactgaaactactggggctacaggtcaggcctcctccctgctgtcaggcaggctcctggaacagaaagctgcgagctgccacaacagctacatcgtcctgtgcattgagaatagcttcatgacctctttctccaaatagtaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggaccacaaggaagtaatccttctgtttctcttgcttctgaaaccaggacaagggaagagagtgtatctgtcagaatgtaagaccggcatcggcaacggctacagaggaacaatgtccaggacaaagagtggtgttgcctgtcaaaagtggggtgccacgttcccccacgtacccaactactctcccagtacacatcccaatgagggactagaagaaaattactgtaggaacccagacaatgatgaacaagggccttggtgctacactacagatccggacaagagatatgactactgcaacattcctgaatgtgaagaagaatgcatgtactgcagtggcgaaaagtatgaggggaaaatctccaagaccatgtctggacttgactgccaggcctgggattctcagagcccacatgctcatggatacatccctgccaaattcccaagcaagaacctgaagatgaattattgccgcaaccctgacggggagccaaggccctggtgcttcacaacagaccccaccaaacgctgggaatactgtgacatcccccgctgcacaacacccccgcccccacccagcccaacctaccaatgtctgaaaggaagaggtgaaaattaccgagggaccgtgtctgtcaccgtgtctgggaaaacctgtcagcgctggagtgagcaaacccctcataggcacaacaggacaccagaaaatttcccctgcaaaaatctggaggagaattactgccggaacccggatggagaaactgctccctggtgctataccactgacagccagctgaggtgggagtactgtgagattccatcctgcgagtcctcagcatcaccagaccagtcagattcctcagttccaccagaggagcaaacacctgtggtccaggaatgctaccagagcgatgggcagagctatcggggtacatcgtccactaccatcacagggaagaagtgccagtcctgggcagctatgtttccacataggcattcgaagacgccagagaacttcccagatgctggcttggagatgaactattgcaggaacccggatggtgacaagggcccttggtgctacaccactgacccgagcgtcaggtgggaatactgcaacctgaagcggtgctcagagacaggagggagtgttgtggaattgcccacagtttcccaggaaccaagtgggccgagcgactctgagacagactgcatgtatgggaatggcaaagactaccggggcaaaacggccgtcactgcagctggcaccccttgccaaggatgggctgcccaggagccccacaggcacagcatcttcaccccacagacaaacccacgggcaggtctggaaaagaattattgccgaaaccccgatggggatgtgaatggtccttggtgctatacaacaaaccctagatgatagctcgagtcaccaggcg

SEQ ID NO: 37 - protein, 101 aa, Homo sapiens
QPVLHLVALNSPLSGGMRGIRGADFQCFQQARAVGLAGTFRAFLSSRLQDLYSIVRRADRAAVPIVNLKDELLFPSWEALFSGSEGPLKPGARIFSFDGKD

SEQ ID NO: 38 - protein, 199 aa, Homo sapiens
SSYVHLRPARPTSPPAHSHRDFQPVLHLVALNSPLSGGMRGIRGADFQCFQQARAVGLAGTFRAFLSSRLQDLYSIVRRADRAAVPIVNLKDELLFPSWEALFSGSEGPLKPGARIFSFDGKDVLRHPTWPQKSVWHGSDPNGRRLTESYCETWRTEAPSATGQASSLLGGRLLGQSAASCHHAYIVLCIENSFMTASK

SEQ ID NO: 39 - protein, 83 aa, Homo sapiens
SECKTGNGKNYRGTMSKTKNGITCQKWSSTSPHRPRFSPATHPSEGLEENYCRNPDNDPQGPWCYTTDPEKRYDYCDILECEE

SEQ ID NO: 40 - protein, 81 aa, Homo sapiens
EECMHCSGENYDGKISKTMSGLECQAWDSQSPHAHGYIPSKFPNKNLKKNYCRNPDRELRPWCFTTDPNKRWELCDIPRCT

SEQ ID NO: 41 - protein, 82 aa, Homo sapiens
YQCLKGTGENYRGNVAVTVSGHTCQHWSAQTPHTHNRTPENFPCKNLDENYCRNPDGKRAPWCHTTNSQVRWEYCKIPSCDS

SEQ ID NO: 42 - protein, 82 aa, Homo sapiens
QDCYHGDGQSYRGTSSTTTTGKKCQSWSSMTPHRHQKTPENYPNAGLTMNYCRNPDADKGPWCFTTDPSVRWEYCNLKKCSG

SEQ ID NO: 43 - protein, 82 aa, Homo sapiens
EDCMFGNGKGYRGKRATTVTGTPCQDWAAQEPHRHSIFTPETNPRAGLEKNYCRNPDGDVGGPWCYTTNPRKLYDYCDVPQC

SEQ ID NO: 44 - protein, 453 aa, Homo sapiens
KVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPR